SEQUENCE LISTING (102 sequences)

SEQ ID NO: 1 - protein, 1295 aa, Escherichia coli
MNKIYSIKYSAATGGLIAVSELAKKVICKTNRKISAALLSLAVISYTNIIYAANMDISKAWARDYLDLAQNKGVFQPGSTHVKIKLKDGTDFSFPALPVPDFSSATANGAATSIGGAYAVTVAHNAKNKSSANYQTYGSTQYTQINRMTTGNDFSIQRLNKYVVETRGADTSFNYNENNQNIIDRYGVDVGNGKKEIIGFRVGSGNTTFSGIKTSQTYQADLLSASLFHITNLRANTVGGNKVEYENDSYFTNLTTNGDSGSGVYVFDNKEDKWVLLGTTHGIIGNGKTQKTYVTPFDSKTTNELKQLFIQNVNIDNNTATIGGGKITIGNTTQDIEKNKNNQNKDLVFSGGGKISLKENLDLGYGGFIFDENKKYTVSAEGNNNVTFKGAGIDIGKGSTVDWNIKYASNDALHKIGEGSLNVIQAQNTNLKTGNGTVILGAQKTFNNIYVAGGPGTVQLNAENALGEGDYAGIFFTENGGKLDLNGHNQTFKKIAATDSGTTITNSNTTKESVLSVNNQNNYIYHGNVDGNVRLEHHLDTKQDNARLILDGDIQANSISIKNAPLVMQGHATDHAIFRTTKTNNCPEFLCGVDWVTRIKNAENSVNQKNKTTYKSNNQVSDLSQPDWETRKFRFDNLNIEDSSLSIARNADVEGNIQAKNSVINIGDKTAYIDLYSGKNITGAGFTFRQDIKSGDSIGESKFTGGIMATDGSISIGDKAIVTLNTVSSLDRTALTIHKGANVTASSSLFTTSNIKSGGDLTLTGATESTGEITPSMFYAAGGYELTEDGANFTAKNQASVTGDIKSEKAAKLSFGSADKDNSATRYSQFALAMLDGFDTSYQGSIKAAQSSLAMNNALWKVTGNSELKKLNSTGSMVLFNGGKNIFNTLTVDELTTSNSAFVMRTNTQQADQLIVKNKLEGANNLLLVDFIEKKGNDKNGLNIDLVKAPENTSKDVFKTETQTIGFSDVTPEIKQQEKDGKSVWTLTGYKTVANADAAKKATSLMSGGYKAFLAEVNNLNKRMGDLRDINGEAGAWARIMSGTGSAGGGFSDNYTHVQVGADNKHELDGLDLFTGVTMTYTDSHAGSDAFSGETKSVGAGLYASAMFESGAYIDLIGKYVHHDNEYTATFAGLGTRDYSSHSWYAGAEVGYRYHVTDSAWIEPQAELVYGAVSGKQFSWKDQGMNLTMKDKDFNPLIGRTGVDVGKSFSGKDWKVTARAGLGYQFDLFANGETVLRDASGEKRIKGEKDGRMLMNVGLNAEIRDNVRFGLEFEKSAFGKYNVDNAINANFRYSF

SEQ ID NO: 2 - protein, 286 aa, Escherichia coli
YKAFLAEVNNLNKRMGDLRDINGEAGAWARIMSGTGSAGGGFSDNYTHVQVGADNKHELDGLDLFTGVTMTYTDSHAGSDAFSGETKSVGAGLYASAMFESGAYIDLIGKYVHHDNEYTATFAGLGTRDYSSHSWYAGAEVGYRYHVTDSAWIEPQAELVYGAVSGKQFSWKDQGMNLTMKDKDFNPLIGRTGVDVGKSFSGKDWKVTARAGLGYQFDLFANGETVLRDASGEKRIKGEKDGRMLMNVGLNAEIRDNVRFGLEFEKSAFGKYNVDNAINANFRYSF

SEQ ID NO: 3 - DNA, 861 bp, Escherichia coli
tataaagccttccttgcagaggtcaacaacctcaacaaacgtatgggtgatctgcgtgacattaacggtgaggccggtgcatgggcccgtatcatgagtggaaccgggtctgccggcggtggattcagtgacaactacacccacgttcaggtcggtgcggataacaaacatgaactcgatggccttgacctcttcaccggggtgaccatgacctataccgacagccatgcaggcagtgatgccttcagtggtgaaacgaagtctgtgggtgccggtctctatgcctctgccatgtttgagtccggagcatatatcgacctcatcggtaagtacgttcaccatgacaacgagtataccgcaactttcgccggccttggcaccagagactacagctcccactcctggtatgccggtgcggaagtcggttaccgttaccatgtaactgactctgcatggattgagccgcaggcggaacttgtttacggtgctgtatccgggaaacagttctcctggaaggaccagggaatgaacctcaccatgaaggataaggactttaatccgctgattgggcgtaccggtgttgatgtgggtaaatccttctccggtaaggactggaaagtcacagcccgcgccggccttggctaccagtttgacctgtttgccaacggtgaaactgtactgcgtgatgcgtccggtgaaaaacgtatcaaaggtgaaaaagacggccgtatgctcatgaatgttggtctgaatgctgagattcgtgacaacgtacgctttggtcttgagtttgagaaatcggcatttggtaagtacaacgtggataacgccatcaacgccaacttccgttactccttctga

SEQ ID NO: 4 - protein, 1299 aa, Escherichia coli
MREYMNKIYSLKYSAATGGLIAVSELAKRVSGKTNRKLVATMLSLAVAGTVNAANIDISNVWARDYLDLAQNKGIFQPGATDVTITLKNGDKFSFHNLSIPDFSGAAASGAATAIGGSYSVTVAHNKKNPQAAETQVYAQSSYRVVDRRNSNDFEIQRLNKFVVETVGATPAETNPTTYSDALERYGIVTSDGSKKIIGFRAGSGGTSFINGESKISTNSAYSHDLLSASLFEVTQWDSYGMMIYKNDKTFRNLEIFGDSGSGAYLYDNKLEKWVLVGTTHGIASVNGDQLTWITKYNDKLVSELKDTYSHKINLNGNNVTIKNTDITLHQNNADTTGTQEKITKDKDIVFTNGGDVLFKDNLDFGSGGIIFDEGHEYNINGQGFTFKGAGIDIGKESIVNWNALYSSDDVLHKIGPGTLNVQKKQGANIKIGEGNVILNEEGTFNNIYLASGNGKVILNKDNSLGNDQYAGIFFTKRGGTLDLNGHNQTFTRIAATDDGTTITNSDTTKEAVLAINNEDSYIYHGNINGNIKLTHNINSQDKKTNAKLILDGSVNTKNDVEVSNASLTMQGHATEHAIFRSSANHCSLVFLCGTDWVTVLKETESSYNKKFNSDYKSNNQQTSFDQPDWKTGVFKFDTLHLNNADFSISRNANVEGNISANKSAITIGDKNVYIDNLAGKNITNNGFDFKQTISTNLSIGETKFTGGITAHNSQIAIGDQAVVTLNGATFLDNTPISIDKGAKVIAQNSMFTTKGIDISGELTMMGIPEQNSKTVTPGLHYAADGFRLSGGNANFIARNMASVTGNIYADDAATITLGQPETETPTISSAYQAWAETLLYGFDTAYRGAITAPKATVSMNNAIWHLNSQSSINRLETKDSMVRFTGDNGKFTTLTVNNLTIDDSAFVLRANLAQADQLVVNKSLSGKNNLLLVDFIEKNGNSNGLNIDLVSAPKGTAVDVFKATTRSIGFSDVTPVIEQKNDTDKATWTLIGYKSVANADAAKKATLLMSGGYKAFLAEVNNLNKRMGDLRDINGESGAWARIISGTGSAGGGFSDNYTHVQVGADNKHELDGLDLFTGVTMTYTDSHAGSDAFSGETKSVGAGLYASAMFESGAYIDLIGKYVHHDNEYTATFAGLGTRDYSSHSWYAGAEVGYRYHVTDSAWIEPQAELVYGAVSGKQFSWKDQGMNLTMKDKDFNPLIGRTGVDVGKSFSGKDWKVTARAGLGYQFDLFANGETVLRDASGEKRIKGEKDGRMLMNVGLNAEIRDNLRFGLEFEKSAFGKYNVDNAINANFRYSF

SEQ ID NO: 5 - protein, 286 aa, Escherichia coli
YKAFLAEVNNLNKRMGDLRDINGESGAWARIISGTGSAGGGFSDNYTHVQVGADNKHELDGLDLFTGVTMTYTDSHAGSDAFSGETKSVGAGLYASAMFESGAYIDLIGKYVHHDNEYTATFAGLGTRDYSSHSWYAGAEVGYRYHVTDSAWIEPQAELVYGAVSGKQFSWKDQGMNLTMKDKDFNPLIGRTGVDVGKSFSGKDWKVTARAGLGYQFDLFANGETVLRDASGEKRIKGEKDGRMLMNVGLNAEIRDNLRFGLEFEKSAFGKYNVDNAINANFRYSF

SEQ ID NO: 6 - DNA, 861 bp, Escherichia coli
tataaagccttccttgctgaggtcaacaaccttaacaaacgtatgggtgatctgcgtgacattaacggtgagtccggtgcatgggcccgaatcattagcggaaccgggtctgccggcggtggattcagtgacaactacacccacgttcaggtcggtgcggataacaaacatgaactcgatggccttgacctcttcaccggggtgaccatgacctataccgacagccatgcaggcagtgatgccttcagtggtgaaacgaagtctgtgggtgccggtctctatgcctctgccatgtttgagtccggagcatatatcgacctcatcggtaagtacgttcaccatgacaacgagtataccgcaactttcgccggccttggcaccagagactacagctcccactcctggtatgccggtgcggaagtcggttaccgttaccatgtaactgactctgcatggattgagccgcaggcggaacttgtttacggtgctgtatccgggaaacagttctcctggaaggaccagggaatgaacctcaccatgaaggataaggactttaatccgctgattgggcgtaccggtgttgatgtgggtaaatccttctccggtaaggactggaaagtcacagcccgcgccggccttggctaccagtttgacctgtttgccaacggtgaaaccgtactgcgtgatgcgtccggtgagaaacgtatcaaaggtgaaaaagacggtcgtatgctcatgaatgttggtctcaacgccgaaattcgcgataatcttcgcttcggtcttgagtttgagaaatcggcatttggtaaatacaacgtggataacgcgatcaacgccaacttccgttactctttctga

SEQ ID NO: 7 - protein, 1300 aa, Escherichia coli
MNKIYSLKYSHITGGLIAVSELSGRVSSRATGKKKHKRILALCFLGLLQSSYSFASQMDISNFYIRDYMDFAQNKGIFQAGATNIEIVKKDGSTLKLPEVPFPDFSPVANKGSTTSIGGAYSITATHNTKNHHSVATQNWGNSTYKQTDWNTSHPDFAVSRLDKFVVETRGATEGADISLSKQQALERYGVNYKGEKKLIAFRAGSGVVSVKKNGRITPFNEVSYKPEMLNGSFVHIDDWSGWLILTNNQFDEFNNIASQGDSGSALFVYDNQKKKWVVAGTVWGIYNYANGKNHAAYSKWNQTTIDNLKNKYSYNVDMSGAQVATIENGKLTGTGSDTTDIKNKDLIFTGGGDILLKSSFDNGAGGLVFNDKKTYRVNGDDFTFKGAGVDTRNGSTVEWNIRYDNKDNLHKIGDGTLDVRKTQNTNLKTGEGLVILGAEKTFNNIYITSGDGTVRLNAENALSGGEYNGIFFAKNGGTLDLNGYNQSFNKIAATDSGAVITNTSTKKSILSLNNTADYIYHGNINGNLDVLQHHETKKENRRLILDGGVDTTNDISLRNTQLSMQGHATEHAIYRDGAFSCSLPAPMRFLCGSDYVAGMQNTEADAVKQNGNAYKTNNAVSDLSQPDWETGTFRFGTLHLENSDFSVGRNANVIGDIQASKSNITIGDTTAYIDLHAGKNITGDGFGFRQNIVRGNSQGETLFTGGITAEDSTIVIKDKAKALFSNYVYLLNTKATIENGADVTTQSGMFSTSDISISGNLSMTGNPDKDNKFEPSIYLNDASYLLTDDSARLVAKNKASVVGDIHSTKSASIMFGHDESDLSQLSDRTSKGLALGLLGGFDVSYRGSVNAPSASATMNNTWWQLTGDSALKTLKSTNSMVYFTDSANNKKFHTLTVDELATSNSAYAMRTNLSESDKLEVKKHLSGENNILLVDFLQKPTPEKQLNIELVSAPKDTNENVFKASKQTIGFSDVTPVITTRETDDKITWSLTGYNTVANKEATRNAAALFSVDYKAFLNEVNNLNKRMGDLRDINGEAGAWARIMSGTGSASGGFSDNYTHVQVGVDKKHELDGLDLFTGFTVTHTDSSASADVFSGKTKSVGAGLYASAMFDSGAYIDLIGKYVHHDNEYTATFAGLGTRDYSTHSWYAGAEAGYRYHVTEDAWIEPQAELVYGSVSGKQFAWKDQGMHLSMKDKDYNPLIGRTGVDVGKSFSGKDWKVTARAGLGYQFDLLANGETVLRDASGEKRIKGEKDSRMLMSVGLNAEIRDNVRFGLEFEKSAFGKYNVDNAVNANFRYSF

SEQ ID NO: 8 - protein, 286 aa, Escherichia coli
YKAFLNEVNNLNKRMGDLRDINGEAGAWARIMSGTGSASGGFSDNYTHVQVGVDKKHELDGLDLFTGFTVTHTDSSASADVFSGKTKSVGAGLYASAMFDSGAYIDLIGKYVHHDNEYTATFAGLGTRDYSTHSWYAGAEAGYRYHVTEDAWIEPQAELVYGSVSGKQFAWKDQGMHLSMKDKDYNPLIGRTGVDVGKSFSGKDWKVTARAGLGYQFDLLANGETVLRDASGEKRIKGEKDSRMLMSVGLNAEIRDNVRFGLEFEKSAFGKYNVDNAVNANFRYSF

SEQ ID NO: 9 - DNA, 861 bp, Escherichia coli
tataaaaattttcttgctgaagtcaacaacctgaacaaacgtatgggtgacctgcgtgacatcaacggcgaagccggtgcatgggcacgcatcatgagcggtaccggctctgccagtggtggtttcagtgacaactacacgcacgttcaggtcggggtcgacaaaaaacatgagctggacggactggatttgtttaccggtttcactgtcacacacactgacagcagtgcctccgccgatgttttcagcggtaaaacgaagtctgtgggggctggcctgtatgcttccgccatgtttgattccggtgcctatatcgacctgattggcaagtatgttcaccatgataatgagtacacagcaacctttgccggactcggaacccgtgattacagcacgcattcatggtatgccggtgcagaagcgggctaccgctatcatgtcactgaggatgcctggattgagccacaggctgagctggtttacggttctgtatccggtaaacagtttgcatggaaggaccagggaatgcatctgtccatgaaggacaaggactacaatccgctgattggccgaaccggtgtagatgtgggtaaatccttctctggtaaggactggaaagtgacagcccgggccggcctgggctaccagttcgacctgctggctaacggcgaaaccgtattgcgggatgcatctggtgaaaaacgcatcaaaggtgaaaaggacagccgtatgctgatgtccgttggcctgaatgcagaaatcagggacaacgtccgctttggactggagtttgagaaatccgcctttggtaagtacaacgttgataatgcagtcaacgctaacttccgttactcgttctga

SEQ ID NO: 10 - protein, 1285 aa, Shigella flexneri
MNKIYSLKYSHITGGLVAVSELTRKVSVGTSRKKVILGIILSSIYGSYGETAFAAMLDINNIWTRDYLDLAQNRGEFRPGATNVQLMMKDGKIFHFPELPVPDFSAVSNKGATTSIGGAYSVTATHNGTQHHAITTQSWDQTAYKASNRVSSGDFSVHRLNKFVVETTGVTESADFSLSPEDAMKRYGVNYNGKEQIIGFRAGAGTTSTILNGKQYLFGQNYNPDLLSASLFNLDWKNKSYIYTNRTPFKNSPIFGDSGSGSYLYDKEQQKWVFHGVTSTVGFISSTNIAWTNYSLFNNILVNNLKKNFTNTMQLDGKKQELSSIIKDKDLSVSGGGVLTLKQDTDLGIGGLIFDKNQTYKVYGKDKSYKGAGIDIDNNTTVEWNVKGVAGDNLHKIGSGTLDVKIAQGNNLKIGNGTVILSAEKAFNKIYMAGGKGTVKINAKDALSESGNGEIYFTRNGGTLDLNGYDQSFQKIAATDAGTTVTNSNVKQSTLSLTNTDAYMYHGNVSGNISINHIINTTQQHNNNANLIFDGSVDIKNDISVRNAQLTLQGHATEHAIFKEGNNNCPIPFLCQKDYSAAIKDQESTVNKRYNTEYKSNNQIASFSQPDWESRKFNFRKLNLENATLSIGRDANVKGHIEAKNSQIVLGNKTAYIDMFSGRNITGEGFGFRQQLRSGDSAGESSFNGSLSAQNSKITVGDKSTVTMTGALSLINTDLIINKGATVTAQGKMYVDKAIELAGTLTLTGTPTENNKYSPAIYMSDGYNMTEDGATLKAQNYAWVNGNIKSDKKASILFGVDQYKEDNLDKTTHTPLATGLLGGFDTSYTGGIDAPAASASMYNTLWRVNGQSALQSLKTRDSLLLFSNIENSGFHTVTVNTLDATNTAVIMRADLSQSVNQSDKLIVKNQLTGSNNSLSVDIQKVGNNNSGLNVDLITAPKGSNKEIFKASTQAIGFSNISPVISTKEDQEHTTWTLTGYKVAENTASSGAAKSYMSGNYKAFLTEVNNLNKRMGDLRDTNGEAGAWARIMSGAGSASGGYSDNYTHVQIGVDKKHELDGLDLFTGLTMTYTDSHASSNAFSGKTKSVGAGLYASAIFDSGAYIDLISKYVHHDNEYSATFAGLGTKDYSSHSLYVGAEAGYRYHVTEDSWIEPQAELVYGAVSGKRFDWQDRGMSVTMKDKDFNPLIGRTGVDVGKSFSGKDWKVTARAGLGYQFDLFANGETVLRDASGEKRIKGEKDGRILMNVGLNAEIRDNLRFGLEFEKSAFGKYNVDNAINANFRYSF

SEQ ID NO: 11 - protein, 286 aa, Shigella flexneri
YKAFLTEVNNLNKRMGDLRDTNGEAGAWARIMSGAGSASGGYSDNYTHVQIGVDKKHELDGLDLFTGLTMTYTDSHASSNAFSGKTKSVGAGLYASAIFDSGAYIDLISKYVHHDNEYSATFAGLGTKDYSSHSLYVGAEAGYRYHVTEDSWIEPQAELVYGAVSGKRFDWQDRGMSVTMKDKDFNPLIGRTGVDVGKSFSGKDWKVTARAGLGYQFDLFANGETVLRDASGEKRIKGEKDGRILMNVGLNAEIRDNLRFGLEFEKSAFGKYNVDNAINANFRYSF

SEQ ID NO: 12 - DNA, 861 bp, Shigella flexneri
tacaaagccttcctgacagaagtcaacaacctgaataaacgaatgggggatctgcgtgacaccaatggcgaggccggtgcatgggcccgcatcatgagcggagcaggttcagcttctggtggatacagtgacaactacacccatgtgcagattggtgtggataaaaaacatgagctggatggacttgaccttttcactggtctgactatgacgtataccgacagtcatgccagcagtaatgcattcagtggcaagacgaagtccgtcggggcaggtctgtatgcttccgctatatttgactctggtgcctatatcgacctgattagtaagtatgttcaccatgataatgagtactcggcgacctttgctggactcggaacaaaagactacagttctcattccttgtatgtgggtgctgaagcaggctaccgctatcatgtaacagaagactcctggattgagccgcaggcagaactggtttatggggccgtatcaggtaaacggttcgactggcaggatcgcggaatgagcgtgaccatgaaggataaggactttaatccgctgattgggcgtaccggtgttgatgtgggtaaatccttctccggtaaggactggaaagtcacagcccgcgccggccttggctaccagtttgacctgtttgccaacggtgaaaccgtactgcgtgatgcgtccggtgagaaacgtatcaaaggtgaaaaagacggtcgtattctcatgaatgttggtctcaacgccgaaattcgcgataatcttcgcttcggtcttgagtttgagaaatcggcatttggtaaatacaacgtggataacgcgatcaacgccaacttccgttactctttctga

SEQ ID NO: 13 - protein, 1305 aa, Escherichia coli
MNKIYALKYCHATGGLIAVSELASRVMKKAARGSLLALFNLSLYGAFLSASQAAQLNIDNVWARDYLDLAQNKGVFKAGATNVSIQLKNGQTFNFPNVPIPDFSPASNKGATTSIGGAYSVTATHNGTTHHAISTQNWGQSSYKYIDRMTNGDFAVTRLDKFVVETTGVKNSVDFSLNSHDALERYGVEINGEKKIIGFRVGAGTTYTVQNGNTYSTGQVYNPLLLSASMFQLNWDNKRPYNNTTPFYNETTGGDSGSGFYLYDNVKKEWVMLGTLFGIASSGADVWSILNQYDENTVNGLKNKFTQKVQLNNNTMSLNSDSFTLAGNNTAVEKNNNNYKDLSFSGGGSINFDNDVNIGSGGLIFDAGHHYTVTGNNKTFKGAGLDIGDNTTVDWNVKGVVGDNLHKIGAGTLNVNVSQGNNLKTGDGLVVLNSANAFDNIYMASGHGVVKINHSAALNQNNDYRGIFFTENGGTLDLNGYDQSFNKIAATDIGALITNSAVQKAVLSVNNQSNYMYHGSVSGNTEINHQFDTQKNNSRLILDGNVDITNDINIKNSQLTMQGHATSHAVFREGGVTCMLPGVICEKDYVSGIQQQENSANKNNNTDYKTNNQVSSFEQPDWENRLFKFKTLNLINSDFIVGRNAIVVGDISANNSTLSLSGKDTKVHIDMYDGKNITGDGFGFRQDIKDGVSVSPESSSYFGNVTLNNHSLLDIGNKFTGGIEAYDSSVSVTSQNAVFDRVGSFVNSSLTLEKGAKLTAQGGIFSTGAVDVKENASLILTGTPSAQKQEYYSPVISTTEGINLGDKASLSVKNMGYLSSDIHAGTTAATINLGDGDAETDSPLFSSLMKGYNAVLSGNITGEQSTVNMNNALWYSDGNSTIGTLKSTGGRVELGGGKDFATLRVKELNANNATFLMHTNNSQADQLNVTNKLLGSNNTVLVDFLNKPASEMNVTLITAPKGSDEKTFTAGTQQIGFSNVTPVISTEKTDDATKWMLTGYQTVSDAGASKTATDFMASGYKSFLTEVNNLNKRMGDLRDTQGDAGVWARIMNGTGSADGGYSDNYTHVQIGADRKHELDGVDLFTGALLTYTDSNASSHAFSGKTKSVGGGLYASALFDSGAYFDLIGKYLHHDNQYTASFASLGTKDYSSHSWYAGAEVGYRYHLSEESWVEPQMELVYGSVSGKSFSWEDRGMALSMKDKDYNPLIGRTGVDVGRTFSGDDWKITARAGLGYQFDLLANGETVLRDASGEKRFEGEKDSRMLMNVGMNAEIKDNMRFGLELEKSAFGKYNVDNAINANFRYSF

SEQ ID NO: 14 - protein, 286 aa, Escherichia coli
YKSFLTEVNNLNKRMGDLRDTQGDAGVWARIMNGTGSADGGYSDNYTHVQIGADRKHELDGVDLFTGALLTYTDSNASSHAFSGKTKSVGGGLYASALFDSGAYFDLIGKYLHHDNQYTASFASLGTKDYSSHSWYAGAEVGYRYHLSEESWVEPQMELVYGSVSGKSFSWEDRGMALSMKDKDYNPLIGRTGVDVGRTFSGDDWKITARAGLGYQFDLLANGETVLRDASGEKRFEGEKDSRMLMNVGMNAEIKDNMRFGLELEKSAFGKYNVDNAINANFRYSF

SEQ ID NO: 15 - DNA, 861 bp, Escherichia coli
tataaatccttcctgacagaggtcaataatctgaacaagcgtatgggtgacctgcgggatactcagggggatgccggcgtctgggcgcgcatcatgaacggtaccggttcggcagatggtggttacagcgataactacactcacgttcagattggtgccgacagaaagcatgagctggacggtgtggatttgttcacgggtgcattactgacctatacagacagcaatgcaagcagccacgccttcagtggtaaaaccaaatccgtggggggagggttgtacgcttcagcactctttgattccggggcttattttgacctgattggtaaatatctccatcacgacaatcagtacacggcgagttttgcgtctcttggtacaaaagactacagctctcattcctggtatgccggtgcagaggtcgggtatcgttaccacctgtcggaagagtcctgggtggagccacagatggagctggtttacggttctgtgtcaggaaaatcttttagctgggaagaccggggaatggccctgagcatgaaagacaaggattataacccactgattggccgtaccggtgttgacgtgggaagaaccttctccggagacgactggaaaattaccgcgcgagccgggctgggttaccagttcgacctgctggcgaacggagaaacggttctgcgggatgcatccggagagaaacgttttgaaggtgaaaaggacagcagaatgctgatgaatgtggggatgaatgcggaaattaaggataatatgcgttttggcttggagctggaaaaatcggcgttcgggaaatataacgtggacaatgcgataaacgctaacttccgttattctttctga

SEQ ID NO: 16 - protein, 1377 aa, Escherichia coli
MNRIYSLRYSAVARGFIAVSEFARKCVHKSVRRLCFPVLLLIPVLFSAGSLAGTVNNELGYQLFRDFAENKGMFRPGATNIAIYNKQGEFVGTLDKAAMPDFSAVDSEIGVATLINPQYIASVKHNGGYTNVSFGDGENRYNIVDRNNAPSLDFHAPRLDKLVTEVAPTAVTAQGAVAGAYLDKERYPVFYRLGSGTQYIKDSNGQLTQMGGAYSWLTGGTVGSLSSYQNGEMISTSSGLVFDYKLNGAMPIYGEAGDSGSPLFAFDTVQNKWVLVGVLTAGNGAGGRGNNWAVIPLDFIGQKFNEDNDAPVTFRTSEGGALEWSFNSSTGAGALTQGTTTYAMHGQQGNDLNAGKNLIFQGQNGQINLKDSVSQGAGSLTFRDNYTVTTSNGSTWTGAGIVVDNGVSVNWQVNGVKGDNLHKIGEGTLTVQGTGINEGGLKVGDGKVVLNQQADNKGQVQAFSSVNIASGRPTVVLTDERQVNPDTVSWGYRGGTLDVNGNSLTFHQLKAADYGAVLANNVDKRATITLDYALRADKVALNGWSESGKGTAGNLYKYNNPYTNTTDYFILKQSTYGYFPTDQSSNATWEFVGHSQGDAQKLVADRFNTAGYLFHGQLKGNLNVDNRLPEGVTGALVMDGAADISGTFTQENGRLTLQGHPVIHAYNTQSVADKLAASGDHSVLTQPTSFSQEDWENRSFTFDRLSLKNTDFGLGRNATLNTTIQADNSSVTLGDSRVFIDKNDGQGTAFTLEEGTSVATKDADKSVFNGTVNLDNQSVLNINDIFNGGIQANNSTVNISSDSAVLGNSTLTSTALNLNKGANALASQSFVSDGPVNISDAALSLNSRPDEVSHTLLPVYDYAGSWNLKGDDARLNVGPYSMLSGNINVQDKGTVTLGGEGELSPDLTLQNQMLYSLFNGYRNIWSGSLNAPDATVSMTDTQWSMNGNSTAGNMKLNRTIVGFNGGTSPFTTLTTDNLDAVQSAFVMRTDLNKADKLVINKSATGHDNSIWVNFLKKPSNKDTLDIPLVSAPEATADNLFRASTRVVGFSDVTPILSVRKEDGKKEWVLDGYQVARNDGQGKAAATFMHISYNNFITEVNNLNKRMGDLRDINGEAGTWVRLLNGSGSADGGFTDHYTLLQMGADRKHELGSMDLFTGVMATYTDTDASADLYSGKTKSWGGGFYASGLFRSGAYFDVIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDSRMLYGVGLNARFGDNTRLGLEVERSAFGKYNTDDAINANIRYSF

SEQ ID NO: 17 - protein, 286 aa, Escherichia coli
YNNFITEVNNLNKRMGDLRDINGEAGTWVRLLNGSGSADGGFTDHYTLLQMGADRKHELGSMDLFTGVMATYTDTDASADLYSGKTKSWGGGFYASGLFRSGAYFDVIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDSRMLYGVGLNARFGDNTRLGLEVERSAFGKYNTDDAINANIRYSF

SEQ ID NO: 18 - DNA, 861 bp, Escherichia coli
tataacaacttcatcactgaagttaacaacctgaacaaacgcatgggcgatttgagggatattaatggcgaagccggtacgtgggtgcgtctgctgaacggttccggctctgctgatggcggtttcactgaccactataccctgctgcagatgggggctgaccgtaagcacgaactgggaagtatggacctgtttaccggcgtgatggccacctacactgacacagatgcgtcagcagacctgtacagcggtaaaacaaaatcatggggtggtggtttctatgccagtggtctgttccggtccggcgcttactttgatgtgattgccaaatatattcacaatgaaaacaaatatgacctgaactttgccggagctggtaaacagaacttccgcagccattcactgtatgcaggtgcagaagtcggataccgttatcatctgacagatacgacgtttgttgaacctcaggcggaactggtctggggaagactgcagggccaaacatttaactggaacgacagtggaatggatgtctcaatgcgtcgtaacagcgttaatcctctggtaggcagaaccggcgttgtttccggtaaaaccttcagtggtaaggactggagtctgacagcccgtgccggcctgcattatgagttcgatctgacggacagtgctgacgttcatctgaaggatgcagcgggagaacatcagattaatggcagaaaagacagtcgtatgctttacggtgtggggttaaatgcccggtttggcgacaatacgcgtttggggctggaagttgaacgctctgcatttggtaaatacaacacagatgatgcgataaacgctaatattcgttattcattctga

SEQ ID NO: 19 - protein, 1376 aa, Escherichia coli
MNKIYALKYCYITNTVKVVSELARRVCKGSTRRGKRLSVLTSLALSALLPTVAGASTVGGNNPYQTYRDFAENKGQFQAGATNIPIFNNKGELVGHLDKAPMVDFSSVNVSSNPGVATLINPQYIASVKHNKGYQSVSFGDGQNSYHIVDRNEHSSSDLHTPRLDKLVTEVAPATVTSSSTADILTPSKYSAFYRAGSGSQYIQDSQGKRHWVTGGYGYLTGGILPTSFFYHGSDGIQLYMGGNIHDHSILPSFGEAGDSGSPLFGWNTAKGQWELVGVYSGVGGGTNLIYSLIPQSFLSQIYSEDNDAPVFFNASSGAPLQWKFDSSTGTGSLKQGSDEYAMHGQKGSDLNAGKNLTFLGHNGQIDLENSVTQGAGSLTFTDDYTVTTSNGSTWTGAGIIVDKDASVNWQVNGVKGDNLHKIGEGTLVVQGTGVNEGGLKVGDGTVVLNQQADSSGHVQAFSSVNIASGRPTVVLADNQQVNPDNISWGYRGGVLDVNGNDLTFHKLNAADYGATLGNSSDKTANITLDYQTHPADVKVNEWSSSNRGTVGSLYIYNNPYTHTVDYFILKTSSYGWFPTGQVSNEHWEYVGHDQNSAQALLANRINNKGYLYHGKLLGNINFSNKATPGTTGALVMDGSANMSGTFTQENGRLTIQGHPVIHASTSQSIANTVSSLGDNSVLTQPTSFTQDDWENRTFSFGSLVLKDTDFGLGRNATLNTTIQADNSSVTLGDSRVFIDKKDGQGTAFTLEEGTSVATKDADKSVFNGTVNLDNQSVLNINDIFNGGIQANNSTVNISSDSAILGNSTLTSTALNLNKGANALASQSFVSDGPVNISDATLSLNSRPDEVSHTLLPVYDYAGSWNLKGDDARLNVGPYSMLSGNINVQDKGTVTLGGEGELSPDLTLQNQMLYSLFNGYRNTWSGSLNAPDATVSMTDTQWSMNGNSTAGNMKLNRTIVGFNGGTSSFTTLTTDNLDAVQSAFVMRTDLNKADKLVINKSATGHDNSIWVNFLKKPSDKDTLDIPLVSAPEATADNLFRASTRVVGFSDVTPTLSVRKEDGKKEWVLDGYQVARNDGQGKAAATFMHISYNNFITEVNNLNKRMGDLRDINGEAGTWVRLLNGSGSADGGFTDHYTLLQMGADRKHELGSMDLFTGVMATYTDTDASAGLYSGKTKSWGGGFYASGLFRSGAYFDLIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDGRMLYGVGLNARFGDNTRLGLEVERSAFGKYNTDDAINANIRYSF

SEQ ID NO: 20 - protein, 286 aa, Escherichia coli
YNNFITEVNNLNKRMGDLRDINGEAGTWVRLLNGSGSADGGFTDHYTLLQMGADRKHELGSMDLFTGVMATYTDTDASAGLYSGKTKSWGGGFYASGLFRSGAYFDLIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDGRMLYGVGLNARFGDNTRLGLEVERSAFGKYNTDDAINANIRYSF

SEQ ID NO: 21 - DNA, 861 bp, Escherichia coli
tataacaacttcatcactgaagttaacaacctgaacaaacgcatgggcgatttgagggatattaacggcgaagccggtacgtgggtgcgtctgctgaacggttccggctctgctgatggcggtttcactgaccactataccctgctgcagatgggggctgaccgtaagcacgaactgggaagtatggacctgtttaccggcgtgatggccacctacactgacacagatgcgtcagcaggcctgtacagcggtaaaacaaaatcatggggtggtggtttctatgccagtggtctgttccggtccggcgcttactttgatttgattgccaaatatattcacaatgaaaacaaatatgacctgaactttgccggagctggtaaacagaacttccgcagccattcactgtatgcaggtgcagaagtcggataccgttatcatctgacagatacgacgtttgttgaacctcaggcggaactggtctggggaagactgcagggccaaacatttaactggaacgacagtggaatggatgtctcaatgcgtcgtaacagcgttaatcctctggtaggcagaaccggcgttgtttccggtaaaaccttcagtggtaaggactggagtctgacagcccgtgccggcctacattatgagttcgatctgacggacagtgctgacgttcacctgaaggatgcagcgggagaacatcagattaatgggagaaaagacggtcgtatgctttacggtgtggggttaaatgcccggtttggcgacaatacgcgtctggggctggaagttgaacgctctgcattcggtaaatacaacacagatgatgcgataaacgctaacattcgttattcattctga

SEQ ID NO: 22 - protein, 1372 aa, Escherichia coli
MNKVYSLKYCPVTGGLIAVSELARRVIKKTCRRLTHILLAGIPAICLCYSQISQAGIVRSDIAYQIYRDFAENKGLFVPGANDIPVYDKDGKLVGRLGKAPMADFSSVSSNGVATLVSPQYIVSVKHNGGYRSVSFGNGKNTYSLVDRNNHPSIDFHAPRLNKLVTEVIPSAVTSEGTKANAYKYTERYTAFYRVGSGTQYTKDKDGNLVKVAGGYAFKTGGTTGVPLISDATIVSNPGQTYNPVNGPLPDYGAPGDSGSPLFAYDKQQKKWVIVAVLRAYAGINGATNWWNVIPTDYLNQVMQDDFDAPVDFVSGLGPLNWTYDKTSGTGTLSQGSKNWTMHGQKDNDLNAGKNLVFSGQNGAIILKDSVTQGAGYLEFKDSYTVSAESGKTWTGAGIITDKGTNVTWKVNGVAGDNLHKLGEGTLTINGTGVNPGGLKTGDGIVVLNQQADTAGNIQAFSSVNLASGRPTVVLGDARQVNPDNISWGYRGGKLDLNGNAVTFTRLQAADYGAVITNNAQQKSQLLLDLKAQDTNVSEPTIGNISPFGGTGTPGNLYSMILNSQTRFYILKSASYGNTLWGNSLNDPAQWEFVGMDKNKAVQTVKDRILAGRAKQPVIFHGQLTGNMDVAIPQVPGGRKVIFDGSVNLPEGTLSQDSGTLIFQGHPVIHASISGSAPVSLNQKDWENRQFTMKTLSLKDADFHLSRNASLNSDIKSDNSHITLGSDRAFVDKNDGTGNYVIPEEGTSVPDTVNDRSQYEGNITLNHNSALDIGSRFTGGIDAYDSAVSITSPDVLLTAPGAFAGSSLTVHDGGHLTALNGLFSDGHIQAGKNGKITLSGTPVKDTANQYAPAVYLTDGYDLTGDNAALEITRGAHASGDIHASAASTVTIGSDTPAELASAETAASAFAGSLLEGYNAAFNGAITGGRADVSMHNALWTLGGDSAIHSLTVRNSRISSEGDRTFRTLTVNKLDATGSDFVLRTDLKNADKINVTEKATGSDNSLNVSFMNNPAQGQALNIPLVTAPAGTSAEMFKAGTRVTGFSRVTPTLHVDTSGGNTKWILDGFKAEADKAAAAKADSFMNAGYKNFMTEVNNLNKRMGDLRDTNGDAGAWARIMSGAGSADGGYSDNYTHVQVGFDKKHELDGVDLFTGVTMTYTDSSADSHAFSGKTKSVGGGLYASALFESGAYIDLIGKYIHHDNDYTGNFASLGTKHYNTHSWYAGAETGYRYHLTEDTFIEPQAELVYGAVSGKTFRWKDGDMDLSMKNRDFSPLVGRTGVELGKTFSGKDWSVTARAGTSWQFDLLNNGETVLRDASGEKRIKGEKDSRMLFNVGMNAQIKDNMRFGLEFEKSAFGKYNVDNAVNANFRYMF

SEQ ID NO: 23 - protein, 286 aa, Escherichia coli
YKNFMTEVNNLNKRMGDLRDTNGDAGAWARIMSGAGSADGGYSDNYTHVQVGFDKKHELDGVDLFTGVTMTYTDSSADSHAFSGKTKSVGGGLYASALFESGAYIDLIGKYIHHDNDYTGNFASLGTKHYNTHSWYAGAETGYRYHLTEDTFIEPQAELVYGAVSGKTFRWKDGDMDLSMKNRDFSPLVGRTGVELGKTFSGKDWSVTARAGTSWQFDLLNNGETVLRDASGEKRIKGEKDSRMLFNVGMNAQIKDNMRFGLEFEKSAFGKYNVDNAVNANFRYMF

SEQ ID NO: 24 - DNA, 861 bp, Escherichia coli
tataaaaacttcatgacggaagttaacaatctgaacaaacgtatgggtgacctgcgtgacacaaacggtgatgccggtgcctgggcgcgcatcatgagtggtgccggttctgcagacggtggttacagtgataattacacccatgttcaggtcggctttgacaaaaaacatgaactggacggtgtggacctgtttaccggtgtcacgatgacctataccgacagcagtgcagacagccatgcattcagcggaaagacgaaatcggtggggggcggtctgtatgcttcagcattgtttgagtccggtgcctatatcgatttgattggtaaatatattcaccatgacaatgattacacaggtaactttgctagcctgggaacgaaacactacaacacccattcctggtatgccggtgctgaaacgggttaccgctatcacctgacagaggacacgttcattgagccgcaggctgaactggtttacggcgccgtgtccgggaaaacattccgctggaaagacggtgatatggacctgagcatgaagaacagggacttcagtccgctggttggaagaacaggggttgaactgggcaagaccttcagtggtaaggactggagtgtgacggcccgtgccggaaccagctggcagtttgacctgctgaataatggagagaccgtactgcgtgatgcgtccggggagaaacggataaaaggagagaaggacagccggatgctgtttaatgttggtatgaatgcgcagataaaggacaatatgcgctttggtctggagtttgagaagtcagcctttggtaaatataacgtggataatgcggtaaacgcgaatttccggtatatgttctga

SEQ ID NO: 25 - protein, 1364 aa, Shigella flexneri
MNKIYYLKYCHITKSLIAVSELARRVTCKSHRRLSRRVILTSVAALSLSSAWPALSATVSAEIPYQIFRDFAENKGQFTPGTTNISIYDKQGNLVGKLDKAPMADFSSATITTGSLPPGDHTLYSPQYVVTAKHVSGSDTMSFGYAKNTYTAVGTNNNSGLDIKTRRLSKLVTEVAPAEVSDIGAVSGAYQAGGRFTEFYRLGGGMQYVKDKNGNRTQVYTNGGFLVGGTVSALNSYNNGQMITAQTGDIFNPANGPLANYLNMGDSGSPLFAYDSLQKKWVLIGVLSSGTNYGNNWVVTTQDFLGQQPQNDFDKTIAYTSGEGVLQWKYDAANGTGTLTQGNTTWDMHGKKGNDLNAGKNLLFTGNNGEVVLQNSVNQGAGYLQFAGDYRVSALNGQTWMGGGIITDKGTHVLWQVNGVAGDNLHKTGEGTLTVNGTGVNAGGLKVGDGTVILNQQADADGKVQAFSSVGIASGRPTVVLSDSQQVNPDNISWGYRGGRLELNGNNLTFTRLQAADYGAIITNNSEKKSTVTLDLQTLKASDINVPVNTVSIFGGRGAPGDLYYDSSTKQYFILKASSYSPFFSDLNNSSVWQNVGKDRNKAIDTVKQQKIEASSQPYMYHGQLNGNMDVNIPQLSGKDVLALDGSVNLPEGSITKKSGTLIFQGHPVIHAGTTTSSSQSDWETRQFTLEKLKLDAATFHLSRNGKMQGDINATNGSTVILGSSRVFTDRSDGTGNAVFSVEGSATATTVGDQSDYSGNVTLENKSSLQIMERFTGGIEAYDSTVSVTSQNAVFDRVGSFVNSSLTLGKGAKLTAQSGIFSTGAVDVKENASLTLTGMPSAQKQGYYSPVISTTEGINLEDNASFSVKNMGYLSSDIHAGTTAATINLGDSDADAGKTDSPLFSSLMKGYNAVLRGSITGAQSTVNMINALWYSDGKSEAGALKAKGSRIELGDGKHFATLQVKELSADNTTFLMHTNNSRADQLNVTDKLSGSNNSVLVDFLNKPASEMSVTLITAPKGSDEKTFTAGTQQIGFSNVTPVISTEKTDDATKWVLTGYQTTADAGASKAAKDFMASGYKSFLTEVNNLNKRMGDLRDTQGDAGVWARIMNGTGSADGDYSDNYTHVQIGVDRKHELDGVDLFTGALLTYTDSNASSHAFSGKNKSVGGGLYASALFNSGAYFDLIGKYLHHDNQHTANFASLGTKDYSSHSWYAGAEVGYRYHLTKESWVEPQIELVYGSVSGKAFSWEDRGMALSMKDKDYNPLIGRTGVDVGRAFSGDDWKITARAGLGYQFDLLANGETVLQDASGEKRFEGEKDSRMLMTVGMNAEIKDNMRLGLELEKSAFGKYNVDNAINANFRYVF

SEQ ID NO: 26 - protein, 286 aa, Shigella flexneri
YKSFLTEVNNLNKRMGDLRDTQGDAGVWARIMNGTGSADGDYSDNYTHVQIGVDRKHELDGVDLFTGALLTYTDSNASSHAFSGKNKSVGGGLYASALFNSGAYFDLIGKYLHHDNQHTANFASLGTKDYSSHSWYAGAEVGYRYHLTKESWVEPQIELVYGSVSGKAFSWEDRGMALSMKDKDYNPLIGRTGVDVGRAFSGDDWKITARAGLGYQFDLLANGETVLQDASGEKRFEGEKDSRMLMTVGMNAEIKDNMRLGLELEKSAFGKYNVDNAINANFRYVF

SEQ ID NO: 27 - DNA, 861 bp, Shigella flexneri
tataagtccttccttacagaggtcaataacctgaacaaacgtatgggtgacctgcgggatactcagggggatgccggtgtctgggcacgcataatgaatggtaccggttcggcagatggtgactacagcgataactacactcacgttcagattggtgtcgacagaaagcatgagctggacggtgtggatttatttacgggggcattgctgacctatacggacagcaatgcaagcagccacgcattcagtggaaaaaacaaatccgtgggtggcggtctgtatgcctctgcactctttaattccggagcttattttgacctgattggtaaatatctccatcatgataatcagcacacggcgaattttgcctcactgggaacaaaagactacagctctcattcctggtatgccggtgctgaagttggttatcgttaccacctgacgaaagagtcctgggtggagccacagatagagctggtttacggttctgtatcaggaaaagcttttagctgggaagaccggggaatggctctgagcatgaaagacaaggattataacccactgattggccgtactggtgttgacgtgggaagagccttctccggagacgactggaaaatcacagctcgagccgggctgggttatcagttcgacctgctggcgaacggagaaacggttctgcaggatgcttccggagagaaacgtttcgaaggtgaaaaagatagcaggatgctgatgacggtagggatgaatgcggaaattaaggataatatgcgtttgggactggagctggagaaatcagcgttcgggaaatataatgtggataatgcgataaacgccaacttccgttatgttttctga

SEQ ID NO: 28 - DNA, 864 bp, Artificial sequence
  Codon optimised sequence for expression in E. coli
tacaaagcgttcctggcggaagttaacaacctgaacaaacgtatgggtgacctgcgtgacatcaacggtgaagcgggtgcgtgggcgcgtatcatgtctggcaccggctcggccggtggtggtttctctgacaactacacccacgttcaggttggtgcggacaacaaacacgaactggacggtctggacctgttcaccggcgttaccatgacctacaccgactctcacgccggctctgacgctttctctggtgaaaccaaatctgttggtgcgggtctgtacgcttctgcgatgtttgaatctggtgcgtacatcgacctgatcggtaaatacgttcaccacgacaacgaatacaccgcgaccttcgcgggtctgggtacccgtgactactcttctcactcttggtacgcgggtgcggaagttggttaccgttaccacgttaccgactctgcgtggatcgaaccgcaggcggaactggtttacggtgcggtttctggtaaacagttctcttggaaagaccagggtatgaacctgaccatgaaagacaaagacttcaacccgctgatcggtcgtaccggcgttgacgtcggtaaatctttctctggtaaagactggaaagttaccgcgcgtgcgggtctgggttaccagttcgacctgttcgctaacggtgaaaccgttctgcgtgacgcttctggtgaaaaacgtatcaaaggtgaaaaagacggtcgtatgctgatgaacgtgggtctgaacgcggaaatccgtgacaacgtgcgtttcggtctggaattcgagaaatctgcgttcggtaaatacaacgtggacaacgcgatcaacgcgaacttccgttactctttctgataa

SEQ ID NO: 29 - protein, 52 aa, Escherichia coli
MNKIYSIKYSAATGGLIAVSELAKKVICKTNRKISAALLSLAVISYTNIIYA

SEQ ID NO: 30 - DNA, 156 bp, Escherichia coli
atgaataaaatatactccattaaatatagtgctgccactggcggactcattgctgtttctgaattagcgaaaaaagtcatatgtaaaacaaaccgaaaaatttctgctgcattattatctctggcagttattagttatactaatataatatatgcc

SEQ ID NO: 31 - DNA, 156 bp, Artificial sequence
  Codon optimised for expression in E. coli
atgaacaaaatctactctatcaaatactctgcggcgaccggcggtctgatcgcggtttctgagctcgccaaaaaagttatctgcaaaaccaaccgtaaaatctctgcggcgctgctgtctctggcggttatctcttacaccaacatcatctacgcg

SEQ ID NO: 32 - protein, 278 aa, Escherichia coli
YKAFLAEVNNLNKRMGDLRDINGEAGAWARIMSGTGSAGGGFSDNYTHVQVGADNKHELDGLDLFTGVTMTYTDSHAGSDAFSGETKSVGAGLYASAMFESGAYIDLIGKYVHHDNEYTRDYSSHSWYAGAEVGYRYHVTDSAWIEPQAELVYGAVSGKQFSWKDQGMNLTMKDKDFNPLIGRTGVDVGKSFSGKDWKVTARAGLGYQFDLFANGETVLRDASGEKRIKGEKDGRMLMNVGLNAEIRDNVRFGLEFEKSAFGKYNVDNAINANFRYSF

SEQ ID NO: 33 - DNA, 35 bp, Artificial sequence
  Primer sequence
atggtaggtctcaaatgaacaaaatctactctatc

SEQ ID NO: 34 - DNA, 37 bp, Artificial sequence
  Primer sequence
gcgcaagcttttatcaatgatgatgatgatgatgacc

SEQ ID NO: 35 - DNA, 29 bp, Artificial sequence
  Primer sequence
tttctgagctcgccaaaaaagttatctgc

SEQ ID NO: 36 - DNA, 64 bp, Artificial sequence
  Primer sequence
ttcgaacttaagagatctaggtgatggtgatggtgatgcgccgcgtagatgatgttggtgtaag

SEQ ID NO: 37 - DNA, 31 bp, Artificial sequence
  Primer sequence
gcgagatctgatgaccgaacagcagtggaac

SEQ ID NO: 38 - DNA, 28 bp, Artificial sequence
  Primer sequence
ttatagatctgatggaagctgctgctgc

SEQ ID NO: 39 - DNA, 32 bp, Artificial sequence
  Primer sequence
ggcgttcgaaaatgaccgaacagcagtggaac

SEQ ID NO: 40 - DNA, 27 bp, Artificial sequence
  Primer sequence
atcctgcagagccgccagcgaacatgc

SEQ ID NO: 41 - DNA, 28 bp, Artificial sequence
  Primer sequence
tatattcgaatcgccgccagcgaacatg

SEQ ID NO: 42 - DNA, 30 bp, Artificial sequence
  Primer sequence
ccaagatctgatgaccgacgtttctcgtaa

SEQ ID NO: 43 - DNA, 30 bp, Artificial sequence
  Primer sequence
ttccttcgaatcgccgccaccagcacccag

SEQ ID NO: 44 - DNA, 28 bp, Artificial sequence
  Primer sequence
taactgcagagccgccaccagcacccag

SEQ ID NO: 45 - DNA, 30 bp, Artificial sequence
  Primer sequence
tgttagatctggaagcggttgaacacttcg

SEQ ID NO: 46 - DNA, 29 bp, Artificial sequence
  Primer sequence
tatgatcgatgttcgcgatgatgtggttg

SEQ ID NO: 47 - DNA, 30 bp, Artificial sequence
  Primer sequence
tatactgcagagttcgcgatgatgtggttg

SEQ ID NO: 48 - DNA, 28 bp, Artificial sequence
  Primer sequence
aataagatctgaacgacgcgcagaccgc

SEQ ID NO: 49 - DNA, 29 bp, Artificial sequence
  Primer sequence
tatattcgaatccgccagtttcgccggag

SEQ ID NO: 50 - DNA, 32 bp, Artificial sequence
  Primer sequence
gccgagatctgaccacctatgatacctggacc

SEQ ID NO: 51 - DNA, 34 bp, Artificial sequence
  Primer sequence
ccgtttcgaatcgccatcgctgttcatcgcaatg

SEQ ID NO: 52 - DNA, 33 bp, Artificial sequence
  Primer sequence
ccggctgcagagccatcgctgttcatcgcaatg

SEQ ID NO: 53 - DNA, 33 bp, Artificial sequence
  Primer sequence
acgtagatctggtttctcagatcgcgaccaccg

SEQ ID NO: 54 - DNA, 35 bp, Artificial sequence
  Primer sequence
tagcctgcagacgcgttagacatgtcaacggtacc

SEQ ID NO: 55 - DNA, 29 bp, Artificial sequence
  Primer sequence
acgtagatctggcgccgtacccggacccg

SEQ ID NO: 56 - DNA, 33 bp, Artificial sequence
  Primer sequence
tagcctgcagaaccacccgccgcgttcaggatc

SEQ ID NO: 57 - DNA, 35 bp, Artificial sequence
  Primer sequence
gccgagatctgatggtttctaaaggtgaagaagac

SEQ ID NO: 58 - DNA, 34 bp, Artificial sequence
  Primer sequence
ccgtctgcagatttatacagttcgtccataccgc

SEQ ID NO: 59 - DNA, 38 bp, Artificial sequence
  Primer sequence
tatgctgcagacacctcttaccagggttctatcaaagc

SEQ ID NO: 60 - DNA, 36 bp, Artificial sequence
  Primer sequence
tatactgcagacaccctgaccgttgacgaactgacc

SEQ ID NO: 61 - DNA, 37 bp, Artificial sequence
  Primer sequence
tatactgcagacaacctgctgctggtcgacttcatcg

SEQ ID NO: 62 - DNA, 34 bp, Artificial sequence
  Primer sequence
tatactgcagacaccccggaaatcaaacagcagg

SEQ ID NO: 63 - DNA, 34 bp, Artificial sequence
  Primer sequence
tatgctgcagactacaaagcgttcctggcggaag

SEQ ID NO: 64 - DNA, 36 bp, Artificial sequence
  Primer sequence
tatgctgcagacggtaacgacaaaaacggtctgaac

SEQ ID NO: 65 - DNA, 35 bp, Artificial sequence
  Primer sequence
tatactgcagacgttaaagcgccggaaaacacctc

SEQ ID NO: 66 - DNA, 36 bp, Artificial sequence
  Primer sequence
tatgctgcagacttcaaaaccgaaacccagaccatc

SEQ ID NO: 67 - DNA, 33 bp, Artificial sequence
  Primer sequence
tatcctgcagacaccggctacaaaaccgttgcg

SEQ ID NO: 68 - DNA, 32 bp, Artificial sequence
  Primer sequence
tatactgcagacaccgaaacccagaccatcgg

SEQ ID NO: 69 - DNA, 34 bp, Artificial sequence
  Primer sequence
tatactgcagacacccagaccatcggtttctctg

SEQ ID NO: 70 - DNA, 36 bp, Artificial sequence
  Primer sequence
catactgcagacaccatcggtttctctgacgttacc

SEQ ID NO: 71 - DNA, 33 bp, Artificial sequence
  Primer sequence
tgtactgcagacggtttctctgacgttaccccg

SEQ ID NO: 72 - DNA, 33 bp, Artificial sequence
  Primer sequence
tctgctgcagactctgacgttaccccggaaatc

SEQ ID NO: 73 - DNA, 38 bp, Artificial sequence
  Primer sequence
ggcactgcagacaaacagcaggaaaaagacggtaaatc

SEQ ID NO: 74 - DNA, 36 bp, Artificial sequence
  Primer sequence
ggcactgcagacaaagacggtaaatctgtttggacc

SEQ ID NO: 75 - DNA, 33 bp, Artificial sequence
  Primer sequence
ggcactgcagacaaatctgtttggaccctgacc

SEQ ID NO: 76 - DNA, 36 bp, Artificial sequence
  Primer sequence
tatactgcagactggaccctgaccggctacaaaacc

SEQ ID NO: 77 - DNA, 30 bp, Artificial sequence
  Primer sequence
ggcactgcagacgcgaacgcggacgcggcg

SEQ ID NO: 78 - DNA, 35 bp, Artificial sequence
  Primer sequence
ggcactgcagacgcgacctctctgatgtctggtgg

SEQ ID NO: 79 - DNA, 36 bp, Artificial sequence
  Primer sequence
ggcacctgcaggctttaaggccggcacccgggtgac

SEQ ID NO: 80 - DNA, 37 bp, Artificial sequence
  Primer sequence
ggcacctgcaggcaccccaaccctgcatgttgatacc

SEQ ID NO: 81 - DNA, 41 bp, Artificial sequence
  Primer sequence
ggcacctgcaggcgatggttttaaagcggaggctgataaag

SEQ ID NO: 82 - DNA, 37 bp, Artificial sequence
  Primer sequence
ggcacctgcaggcgctgacagtttcatgaatgccggg

SEQ ID NO: 83 - DNA, 40 bp, Artificial sequence
  Primer sequence
ggcacctgcaggctataaaaacttcatgacggaagttaac

SEQ ID NO: 84 - DNA, 35 bp, Artificial sequence
  Primer sequence
gcgcaagctttcagaacatataccggaaattcgcg

SEQ ID NO: 85 - DNA, 20 bp, Artificial sequence
  Primer sequence
gagttattttaccactccct

SEQ ID NO: 86 - DNA, 17 bp, Artificial sequence
  Primer sequence
cgcagtagcggtaaacg

SEQ ID NO: 87 - DNA, 34 bp, Artificial sequence
  Primer sequence
gacgttaccccggaagcgaaacagcaggaaaaag

SEQ ID NO: 88 - DNA, 34 bp, Artificial sequence
  Primer sequence
ctttttcctgctgtttcgcttccggggtaacgtc

SEQ ID NO: 89 - DNA, 34 bp, Artificial sequence
  Primer sequence
gacgttaccccggaaaaaaaacagcaggaaaaag

SEQ ID NO: 90 - DNA, 34 bp, Artificial sequence
  Primer sequence
ctttttcctgctgttttttttccggggtaacgtc

SEQ ID NO: 91 - DNA, 36 bp, Artificial sequence
  Primer sequence
aaagacggtaaatctgttgcgaccctgaccggctac

SEQ ID NO: 92 - DNA, 36 bp, Artificial sequence
  Primer sequence
gtagccggtcagggtcgcaacagatttaccgtcttt

SEQ ID NO: 93 - DNA, 36 bp, Artificial sequence
  Primer sequence
aaagacggtaaatctgttaaaaccctgaccggctac

SEQ ID NO: 94 - DNA, 36 bp, Artificial sequence
  Primer sequence
gtagccggtcagggttttaacagatttaccgtcttt

SEQ ID NO: 95 - DNA, 35 bp, Artificial sequence
  Primer sequence
gtaaatctgtttggaccgcgaccggctacaaaacc

SEQ ID NO: 96 - DNA, 35 bp, Artificial sequence
  Primer sequence
ggttttgtagccggtcgcggtccaaacagatttac

SEQ ID NO: 97 - DNA, 35 bp, Artificial sequence
  Primer sequence
gtaaatctgtttggaccaaaaccggctacaaaacc

SEQ ID NO: 98 - DNA, 35 bp, Artificial sequence
  Primer sequence
ggttttgtagccggttttggtccaaacagatttac

SEQ ID NO: 99 - DNA, 35 bp, Artificial sequence
  Primer sequence
ctgtttggaccctgaccgcgtacaaaaccgttgcg

SEQ ID NO: 100 - DNA, 35 bp, Artificial sequence
  Primer sequence
cgcaacggttttgtacgcggtcagggtccaaacag

SEQ ID NO: 101 - DNA, 35 bp, Artificial sequence
  Primer sequence
ctgtttggaccctgaccaaatacaaaaccgttgcg

SEQ ID NO: 102 - DNA, 35 bp, Artificial sequence
  Primer sequence
cgcaacggttttgtatttggtcagggtccaaacag

(19) United States
(12) Patent Application Publication
(10) Pub. No.: US 2013/0344536 A1
(43) Pub. Date: Dec. 26, 2013

(54) PROTEIN SECRETION

(71) Applicants: Yanina Romanovna Sevastsyanovich, Edgbaston (GB); Denise Lorena Leyton, Edgbaston (GB); Ian Robert Henderson, Edgbaston (GB)

(72) Inventors: Yanina Romanovna Sevastsyanovich, Edgbaston (GB); Denise Lorena Leyton, Edgbaston (GB); Ian Robert Henderson, Edgbaston (GB)

(21) Appl. No.: 13/985,546

(22) PCT Filed: Feb. 16, 2012
(86) PCT No.: PCT/GB2012/050352

§ 371 Date: Sep. 16, 2013

(30) Foreign Application Priority Data
Feb. 16, 2011 (GB) 1102700.0

(51) Int. Cl. C12P 21/00

(52) U.S. Cl. CPC C12P 21/00
USPC 435/69.1; 435/320.1; 530/350; 536/23.1

(57) ABSTRACT

A bacterial expression construct comprises a nucleic acid sequence encoding a secretion unit peptide comprising less than 300 amino acids of the C-terminus of a SPATE-class bacterial autotransporter polypeptide, the secretion unit peptide 5 comprising: (i) the α-helix; (ii) linker; and (iii) β-barrel region of the β-domain of the autotransporter polypeptide. Such an expression construct, and associated nucleic acids and peptides, find application in the expression of proteins of interest from a host bacterial cell to the cell culture medium.

The present invention concerns bacterial protein expression constructs, peptides which can be used to direct secretion of proteins of interest from the cell to the cell culture medium, and associated nucleic acids and peptides.

The International Biopharmaceutical Association states that in 2003 the biopharmaceutical industry employed more than 2.7 million people and generated $172 billion in real output. The current projection is that by 2014 the total employment impact will increase to over 3.6 million and the real output figure will reach $350.1 billion. The biopharmaceutical industry relies on recombinant protein production (RPP). However, there are several barriers to RPP: (1) Biological products are complex molecules which often require slow, complex manufacturing methods and a battery of analytical techniques; thus, there is a need for new tools and methods which will accelerate development; (2) the demand for products such as monoclonal antibodies is driving the need to improve efficiency; and (3) the complexity of biomolecules presents a challenge in terms of controlling the effect process conditions have on product purity and heterogeneity. Research to overcome these barriers is a priority.

It is desirable for bacterial protein expression systems to provide a number of key characteristics for it to be useful for the industrial scale preparation of recombinant proteins of interest: the system should be easily manipulated by standard molecular biology techniques; it should be capable of using commonly used bacterial strains for industrial scale preparation; the system should pose minimal restriction on the size of the recombinant protein of interest; it should require minimal addition of amino acids to the recombinant protein of interest to effect secretion; the system should cause negligible detrimental effects on host cell viability and integrity; it should produce sufficiently large amounts of the target protein to be commercially viable; and the recombinant protein of interest should be produced in a manner allowing it to be isolated with minimal process impurities.

A bacterial expression system in which the recombinant protein of interest is correctly folded and secreted into the culture fluid, with minimal addition of extra amino acids would: (1) remove the need for elaborate extraction techniques; (2) significantly reduce the diversity and quantity of process impurities; (3) reduce the size and/or number of downstream processing (DSP) unit operations; (4) increase the overall process robustness; (5) speed-up the process development time, (6) reduce the development and manufacturing costs whilst; and (7) speed up the time-to-market for the protein.

For a variety of reasons the host cell of choice for the production of biopharmaceuticals, and other recombinant proteins of interest, is E. coli, with proteins being targeted to the cytoplasm or periplasm. The production of such recombinant proteins is rarely limited by the ability to clone and express a particular gene encoding a protein of interest: substantial bottlenecks arise from protein folding, post-translational modifications and secretion. Recombinant proteins overexpressed in the E. coli cytoplasm often accumulate in a misfolded form as ‘inclusion bodies’, rather than in a correctly-folded form. In contrast, accumulation of periplasmically-targeted proteins often adversely affects bacterial growth and viability. However, in both cases mechanical or chemical extraction techniques must be employed to release the target protein from the host cells. These processes are associated with numerous ‘process impurities’, such as host cell proteins, DNA, endotoxin and the whole cell itself or cellular fragments, and ‘product impurities’ such as aggregates, oxidised forms or non-functional forms of the target proteins.

Against this background, the present inventors have investigated developing a protein expression system in which the protein of interest is secreted from the bacterial cell to the culture medium.

E. coli and other Gram-negative bacteria are characterised in having a double layer of cell membrane: the inner cytoplasmic membrane and the external outer membrane. The space between the inner and outer membranes is the periplasmic space, or periplasm. E. coli and other Gram-negative bacteria secrete few proteins, as the existence of the outer-membrane poses a barrier to the release of the protein of interest into the extracellular milieu. To overcome the outer-membrane barrier and achieve extracellular localisation of a target protein at efficient levels, one of the Gram-negative outer membrane secretion systems can be utilised to try and drive secretion of the protein of interest.

The molecular analysis of the protein secretion pathways of Gram-negative bacteria has revealed the existence of at least seven major, distinct and conserved mechanisms of protein secretion. These pathways are functionally independent mechanisms with respect to outer membrane translocation; commonalities exist in the inner membrane transport steps of some systems. These pathways have been numbered Type I, II, III, IV, V, VI and the Chaperone-Usher pathways.

Most attempts at commercial production of extracellular proteins have failed. The bacterial chaperone-usher and Type I-III protein secretion systems have all been adapted to translocate foreign proteins to the surface of cells or into the extracellular milieu. However, these have not met with commercial success for a number of different reasons, but mainly the complexity of these systems (consisting of 3-20 different subunits) makes it difficult to secrete non-native proteins. An additional factor is that proteins targeted via the Type I and III systems possess targeting signals for their secretion machineries that are not cleaved.

The autotransporter (AT) protein secretion pathway falls under the umbrella of Type V secretion. In contrast to the other secretion systems that are composed of multiple subunits, ranging from 3 to >20 different proteins, ATs are encoded as single polypeptides. Gram-negative bacteria utilise the AT system to secrete a wide variety of different functional moieties with a wide range in size for the translocated passenger domain (20-500 kDa).

The AT protein secretion pathway has been identified in a range of different Gram-negative bacterial species. In all cases, the structure of AT polypeptide is conserved, superficially consisting of three distinct domains: (i) the N-terminal signal sequence; (ii) the functional ‘passenger’ domain; and (iii) the C-terminal β-domain. ATs are first translocated across the inner membrane via a widespread periplasmic targeting signal sequence peptide. After export through the inner membrane, the signal sequence peptide is removed and the remainder of the AT protein is released into the periplasm. The C-terminal β-domain adopts a characteristic β-barrel structure which inserts into the outer membrane of the bacterial cell. The ‘passenger’ domain of the AT polypeptide is then translocated to the cell surface via the pore of the β-barrel structure. Once extruded, the passenger domain adopts its native conformation on the cell surface with the functional domain located N-proximally.

After extrusion to the cell surface, the ‘passenger’ domain may either remain covalently attached to the β-domain as an intact outer membrane protein, or may be cleaved into separate ‘passenger’ and translocation unit domains. Cleaved passenger domains may be released into the extracellular milieu. In some cases, passenger domain cleavage is autoproteolytic.

Accordingly the AT secretion system can be harnessed to display a wide variety of functionally distinct recombinant proteins on the cell surface, using standard molecular biology techniques to replace the DNA encoding the native passenger domain with sequences encoding the protein of interest. The AT secretion system allows a large number of native molecules (as many as 10⁵/cell) to be inserted into the outer membrane without hampering cell viability or reducing cell integrity.

In addition, the use of the AT to secrete a protein of interest from the bacterial cell and release it to the culture medium has also been investigated. However, the use of AT for this purpose is limited due to the mechanisms by which the passenger domains are cleaved from the β-domain. Thus, virtually all of the work done to date has focussed on surface display of the secreted target protein, rather than release into the extracellular milieu.

Against this background the present inventors have investigated harnessing the AT polypeptide system to direct secretion of proteins of interest from the bacterial cell, and subsequent release of those proteins from the AT polypeptide to the culture medium. They have determined the minimal fragment of the β-domain from SPATE-class AT polypeptides, termed the “secretion unit”, that is sufficient to direct secretion and release of proteins from the host cell.

Accordingly a first aspect of the invention provides a bacterial expression construct comprising a nucleic acid sequence encoding a secretion unit peptide comprising less than 300 amino acids of the C-terminus of a SPATE-class bacterial autotransporter polypeptide, said secretion unit peptide comprising: (i) the α-helix; (ii) linker; and (iii) β-barrel region of the β-domain of the autotransporter polypeptide.

To be an effective system for recombinant protein production an Autotransporter system needs to undergo autoprocessing, where the recombinant target protein is released into the extracellular milieu. The inventors have devised a system that effects protein secretion into the culture supernatant in a soluble form. This system is based on the SPATE-class of AT polypeptides. The inventors have used the Pet and Pic AT polypeptides as examples of that class.

Pet is an enterotoxin secreted by enteroaggregative E. coli and belongs to a subgroup of the Autotransporters termed the SPATEs (serine protease autotransporters of the Enterobacteriaceae). Pet carries an N-terminal signal sequence required for protein transport through inner membrane in a SecB-dependent manner, a passenger domain where the effector function (serine protease) is encoded, and a C-terminal β-barrel that mediates passenger domain translocation to the cell surface.

Unlike many other autotransporters that remain attached to its β-barrel or associated with the outer membrane, for SPATE-class ATs, including Pet, the passenger domain is cleaved off and secreted into extracellular environment. Due to this property, together with the apparent simplicity of the autotransporter secretion mechanism, SPATE-class ATs can be exploited for secretion of soluble recombinant proteins into the culture medium.

However, to date the minimum length of amino acids from SPATE ATs required for effective secretion and release of soluble recombinant proteins has not been determined. As stated above, it is desirable to minimise the region of amino acids added to the recombinant protein of interest to effect secretion. This is because the more amino acids that are added to the recombinant protein of interest, then the more likely it is that the added amino acids will affect the function of the recombinant protein of interest, or the biocompatibility of the recovered protein.

The present inventors have determined that a “secretion unit” peptide of less than 300 amino acids, said secretion unit comprising: (i) the α-helix; (ii) linker; and (iii) β-barrel region of the β-domain of a SPATE-class bacterial autotransporter polypeptide can be effectively harnessed to secrete a protein of interest from the bacterial cell and mediate its release into the culture medium.

Importantly, the ‘secretion unit peptide’ does not have to include any amino acid sequence from the ‘passenger domain’ (where the ‘passenger domain’ includes the functional portion of the protein, the autochaperone domain (AC) and the hydrophobic secretion facilitator, which the inventors have termed ‘HSF’) of a SPATE-class bacterial autotransporter polypeptide in order to direct efficient secretion and release of a protein of interest into the culture medium.

This finding is surprising and unexpected. In recent studies of SPATE-class AT polypeptides, it has been concluded that additional amino acids from the ‘autochaperone’ (AC) region of the passenger domain of the AT polypeptides are required to effectively secrete a protein of interest from the bacterial cell and mediate its release into the culture medium. For example, Soprova et al (2010) J. Biol Chem 285, 38224-38233 concludes that amino acid residues in the AC region of the autotransporter hemoglobin protease (Hbp) are necessary for translocation of the AT. Binder et al (2010) J. Mol. Biol 400, 783-802, also conclude that a region from the passenger domain of SPATE-class AT polypeptides, the HSF domain, is required for correct display of the protein on the cell surface. Jong et al (2010) Curr. Opin. Biotech 21, 646-652 review recent progress towards harnessing ATs for the secretion of protein into culture medium or display on the cell surface. The document also reports that proteins of interest are fused to the autochaperone region of the passenger domain, and that the autochaperone domain is important for efficient translocation through the outer membrane. Peterson et al (2010) PNAS 107, 17739-17744 reports that a fragment of the passenger domain of EspP, a SPATE-class AT polypeptide, is required for efficient translocation of the passenger domain.

Hence, until the present invention, it was the consensus of opinion in this field of research that a portion of the “passenger domain” of the AT polypeptide had to be retained and fused with the protein of interest to ensure that a protein of interest is secreted from a host bacterial cell and released into the culture medium

The present inventors have demonstrated that this is not correct. The ‘secretion unit peptide’ does not have to include any amino acid sequence from the ‘passenger domain’ of a SPATE-class bacterial autotransporter polypeptide. Therefore, the aspects of the present invention provided herein are based on the surprising finding that a “secretion unit” comprising less than 300 amino acids of the C-terminus of a SPATE-class bacterial autotransporter polypeptide, said secretion unit peptide comprising: (i) the α-helix; (ii) linker; and (iii) β-barrel region of the β-domain of the autotransporter polypeptide is sufficient for this purpose.

An embodiment of the invention is wherein the secretion unit peptide does not include any amino acid sequence from the ‘passenger domain’ of a SPATE-class bacterial autotransporter polypeptide.

As stated above, the first aspect of the invention provides a bacterial expression construct. The expression construct is used for the efficient expression and secretion of a protein of interest from a bacterial cell to the extracellular milieu. In use, a gene encoding a protein of interest is cloned in to the expression construct such that the gene is operatively linked with the nucleic acid sequence encoding a secretion unit peptide. Upon introduction of the bacterial expression construct into an appropriate host cell, for example a Gram-negative bacterium such as E. coli, the protein of interest and the secretion unit peptide are formed as a single fusion polypeptide molecule.

On translocation of the fusion polypeptide molecule to the periplasm, the secretion unit peptide component of the fusion polypeptide mediates both the translocation of the protein of interest through the outer membrane, and its release from the fusion polypeptide into the cell culture medium. Once released, the protein of interest can be recovered from the cell culture medium using standard techniques in the art. Accordingly, the present invention provides a bacterial expression construct and associated peptide and nucleic acid molecules that have much utility for the preparation of proteins of interest.

By “bacterial expression construct”, the construct is based on expression constructs known in the art that can be used to direct the expression of recombinant polypeptides in bacterial host cells.

An “expression construct” is a term well known in the art. Expression constructs are basic tools for biotechnology and the production of proteins. It generally includes a plasmid that is used to introduce a specific gene into a target cell, a “host cell”. Once the expression construct is inside the cell, protein that is encoded by that gene is produced by the cellular-transcription and translation machinery ribosomal complexes. The plasmid also includes nucleic acid sequences required for maintenance and propagation of the vector. The goal of an expression vector is the production of large amounts of stable messenger RNA, and therefore proteins.

Suitable expression constructs comprising nucleic acid for introduction into bacteria can be chosen or constructed, containing appropriate regulatory sequences, including promoter sequences, terminator fragments, enhancer sequences, marker genes and other sequences as appropriate. For further details see, for example, Molecular Cloning: a Laboratory Manual: 2nd edition, Sambrook et al., 1989, Cold Spring Harbor Laboratory Press.

The plasmid is frequently engineered to contain regulatory sequences that act as enhancer and promoter regions and lead to efficient transcription of the gene carried on the expression vector. Most parts of the regulatory unit are located upstream of coding sequence of the heterologous gene and are operably linked thereto. The expression cassette may also contain a downstream 3′ untranslated region comprising a polyadenylation site. The regulatory sequences can direct constitutive or inducible expression of the heterologous coding sequence.

As an example, the expression construct can be based on the generic pASK-IBA33plus expression vector; expression of the recombinant protein can be induced from the tet promoter/operator in E. coli TOP10 strain.

By “protein of interest”, or other such terms like “recombinant protein”, “heterologous protein”, “heterologous coding sequence”, “heterologous gene sequence”, “heterologous gene”, “recombinant gene” or “gene of interest”, as can be used are interchangeably herein, these terms refer to a protein product that is sought to be expressed in the mammalian cell and harvested in high amount, or nucleic acid sequences that encode such a protein. The product of the gene can be a protein or polypeptide, but also a peptide.

The protein of interest may be any protein of interest, e.g. a therapeutic protein such as an interleukin or an enzyme or a subunit of a multimeric protein such as an antibody or a fragment thereof, as can be appreciated by the skilled person.

For the avoidance of doubt, the bacterial expression construct of the first aspect of the invention can comprise more than one nucleic acid encoding a protein of interest.

The bacterial expression construct of the first aspect of the invention comprises nucleic acid sequence encoding a secretion unit peptide comprising less than 300 amino acids of the C-terminus of a SPATE-class bacterial autotransporter polypeptide. By “secretion unit peptide” we mean that the peptide comprises the α-helix, linker, and (3-barrel region of the β-domain of a SPATE-class bacterial autotransporter polypeptide.

The β-domain of a SPATE-class bacterial autotransporter polypeptide has been well characterised. The specific sub-regions of the β-domain, i.e. α-helix, linker, and (3-barrel region, are terms which are well known in the art.

For the avoidance of doubt, the “secretion unit peptide” encoded by the nucleic acid sequence within the bacterial expression construct of the first aspect of the invention does not include peptides that are derived from AT polypeptides that have been altered such that they are not capable of translocating a linked protein of interest.

Many SPATE-class bacterial AT polypeptides are known. Thus, the secretion unit peptide may comprise less than 300 amino acids of the C-terminus of any SPATE-class bacterial autotransporter polypeptide. Examples of different types of SPATE AT polypeptides include Pet, Sat, EspP, SigA, EspC, Tsh, SepA, Pic, Hbp, SsaA, EatA, EpeA, EspI, PicU, Vat, Boa, IgA1, Hap, App, MspA, EaaA and EaaC, as well as further homologous polypeptides. Hence an embodiment of the first aspect of the invention is wherein the secretion unit peptide is derivable from one of these AT polypeptides or a homologous polypeptide whose secretion unit possesses the same function.

By “homologous polypeptide” we mean a polypeptide having an amino acid sequence that has a similarity or identity of at least 30%, at least 40%, at least 50%, at least 60%, at least 70%, at least 80%, at least 85%, at least 90%, at least 95%, at least 97%, at least 98% or at least 99% to a known SPATE-class bacterial AT polypeptide, including those listed above.

In an embodiment, the SPATE-class bacterial AT polypeptide comprises less than 300 amino acids of the C-terminus of Pet, Sat, EspP, SigA, EspC, Tsh, SepA or Pic.

Examples of each type of SPATE-class bacterial autotransporter polypeptide are well known in the art. An embodiment of the present invention is wherein the secretion unit peptide is derivable from one or more SPATE-class bacterial AT polypeptides selected from the following: PET_ECO44, SAT_CFT073, ESPP_ECO57, SIGA_SHIFL, ESPC_ECO27, TSH_{—}E. coli, SEPA_EC536, PIC_ECO44, SEPA_SHIFL.

By “derivable” we include where the secretion unit peptide is encoded by a nucleic acid sequence derived from a larger section of nucleic acid which encodes the particular SPATE-class bacterial AT polypeptide. Alternatively, the secretion unit peptide may be encoded by a nucleic acid sequence synthesised de novo having the desired nucleotide sequence.

For the Pet AT polypeptide the α-helix region is located from 1010 to 1024; the linker region is located from 1025 to 1033; and the β-barrel region from 1034 to 1295, of the amino acid sequence shown in SEQ ID NO:1 at the end of the description. Further information may be found in GenBank accession number FN554767.1 (Swiss-Prot accession number O68900.1). Representative amino acid sequence from 1010 to 1295 of Pet AT polypeptide PET_ECO44 is provided in SEQ ID NO:2 at the end of the description. Representative nucleic acid sequence encoding the secretion unit of PET_ECO44 is provided in SEQ ID NO:3 at the end of the description.

For the Sat AT polypeptide the α-helix region is located from 1014 to 1028; the linker region is located from 1029 to 1037; and the β-barrel region from 1038 to 1299, of the amino acid sequence shown in SEQ ID NO:4 at the end of the description. Further information may be found in GenBank accession number AAN82067.1. Representative amino acid sequence from 1014 to 1299 of SAT_CFT073 polypeptide is provided in SEQ ID NO:5 at the end of the description. Representative nucleic acid sequence encoding the secretion unit of SAT_CFT073 is provided in SEQ ID NO:6 at the end of the description.

For the EspP polypeptide the α-helix region is located from 1015 to 1029; the linker region is located from 1030 to 1038; and the β-barrel region from 1039 to 1300, of the amino acid sequence shown in SEQ ID NO:7 at the end of the description. Further information may be found in Swiss-Prot accession number Q7BSW5.1. Representative amino acid sequence from 1015 to 1300 of EspP polypeptide ESPP_ECO57 is provided in SEQ ID NO:8 at the end of the description. Representative nucleic acid sequence encoding the secretion unit of ESPP_ECO57 is provided in SEQ ID NO:9 at the end of the description.

For the SigA AT polypeptide the α-helix region is located from 1000 to 1014; the linker region is located from 1015 to 1023; and the β-barrel region from 1024 to 1285, of the amino acid sequence shown in SEQ ID NO:10 at the end of the description. Further information may be found in GenBank accession number: AAF67320.1. Representative amino acid sequence from 1000 to 1285 of SigA AT polypeptide SIGA_SHIFL is provided in SEQ ID NO:11 at the end of the description. Representative nucleic acid sequence encoding the secretion unit of SIGA_SHIFL is provided in SEQ ID NO:12 at the end of the description.

For the EspC AT polypeptide the α-helix region is located from 1020 to 1035; the linker region is located from 1035 to 1043; and the β-barrel region from 1044 to 1305, of the amino acid sequence shown in SEQ ID NO:13 at the end of the description. Further information may be found in Swiss-Prot accession number Q9EZE7.2. Representative amino acid sequence from 1020 to 1305 of EspC AT polypeptide ESPC_ECO27 is provided in SEQ ID NO:14 at the end of the description. Representative nucleic acid sequence encoding the secretion unit of ESPC_ECO27 is provided in SEQ ID NO: 15 at the end of the description.

For the Tsh AT polypeptide the α-helix region is located from 1092 to 1106; the linker region is located from 1107 to 1115; and the β-barrel region from 1116 to 1377, of the amino acid sequence shown in SEQ ID NO:16 at the end of the description. Further information may be found in GenBank accession number AAA24698.1. Representative amino acid sequence from 1092 to 1377 of the Tsh AT polypeptide TSH_{—}E. coli is provided in SEQ ID NO:17 at the end of the description. Representative nucleic acid sequence encoding the secretion unit of TSH_{—}E. coli is provided in SEQ ID NO:18 at the end of the description.

For the SepA AT polypeptide the α-helix region is located from 1091 to 1105; the linker region is located from 1006 to 1114; and the β-barrel region from 1115 to 1376, of the amino acid sequence shown in SEQ ID NO:19 at the end of the description. Further information may be found in NCBI Reference Sequence: YP_{—}668278.1. Representative amino acid sequence from 1091 to 1376 of the SepA AT polypeptide SEPA_EC536 is provided in SEQ ID NO:20 at the end of the description. Representative nucleic acid sequence encoding the secretion unit of SEPA_EC536 is provided in SEQ ID NO:21 at the end of the description.

For the Pic AT polypeptide the α-helix region is located from 1087 to 1101; the linker region is located from 1102 to 1110; and the β-barrel region from 1111 to 1372, of the amino acid sequence shown in SEQ ID NO:22 at the end of the description. Further information may be found in Swiss-Prot accession number Q7BS42.2. Representative amino acid sequence from 1087 to 1372 of the Pic AT polypeptide PIC_ECO44 is provided in SEQ ID NO:23 at the end of the description. Representative nucleic acid sequence encoding the secretion unit of PIC_ECO44 is provided in SEQ ID NO:24 at the end of the description.

Also, an additional example of the SepA AT polypeptide is provided in SEQ ID NO: 25. This is the SEPA_SHIFL AT polypeptide. The α-helix region is located from 1081 to 1095; the linker region is located from 1096 to 1104 and the β-barrel region from 1105 to 1364, of the amino acid sequence shown in SEQ ID NO:25 at the end of the description. Further information may be found in Swiss-Prot accession number Q8VSL2.1. Representative amino acid sequence from 1079 to 1364 of SEPA_SHIFL is provided in SEQ ID NO:26 at the end of the description. Representative nucleic acid sequence encoding the secretion unit of SEPA_SHIFL is provided in SEQ ID NO:27 at the end of the description.

It can be appreciated that the nucleic acid encoding the secretion unit peptide can encode amino acid sequence derived from a single SPATE-class AT. For example, the nucleic acid sequence can encode amino acids 1010 to 1295 of the Pet AT PET_ECO44 as discussed above. Alternatively, the nucleic acid sequence can encode different regions of the secretion unit derived from different SPATE-class ATs. For example, the nucleic acid sequence could encode amino acids 1010 to 1024 of the Pet AT PET_ECO44, then the linker region from 1029 to 1037 of SAT_CFT073, followed by the β-barrel region from 1039 to 1300 of EspP polypeptide ESPP_ECO57. This “mixing” of regions of amino acids to provide a secretion unit peptide is an embodiment of the invention.

However, a preferred embodiment of the first aspect of the invention is wherein the secretion unit comprises the amino acid sequence provided in any one of SEQ ID NOs 2, 5, 8, 11, 14, 17, 20, 23 or 26 or a variant thereof, wherein the variant is capable of mediating the extracellular secretion of a peptide from the periplasm.

The term “variant” as used herein used to describe a secretion unit peptide which retains the biological function of that peptide, i.e. it is capable of mediating the extracellular secretion and release of a protein of interest. As shown herein, using said secretion unit peptide in a fusion protein increases the secretion of said protein from a bacterial cell. A skilled person would know that the sequence of any one of SEQ ID NOs 2, 5, 8, 11, 14, 17, 20, 23 or 26 can be altered without the loss of biological activity. In particular, single like for like changes with respect to the physio-chemical properties of the respective amino acid should not disturb the functionality, and moreover small deletions within non-functional regions of the secretion unit peptide can also be tolerated and hence are considered “variants” for the purpose of the present invention. The experimental procedures described below can be readily adopted by the skilled person to determine whether a ‘variant’ can still function as a secretion unit peptide, i.e. whether the variant is capable of mediating the extracellular secretion and release of a protein of interest.

Also, the β-barrel region of the secretion unit peptide includes two types of structural amino acid motifs: the β-strands which are inserted in to the extracellular membrane, and the surface loops which as positioned between the β-strands and are located in the extracellular milieu. The present inventors have shown it is possible to alter or remove amino acid sequence of the surface loops within the β-barrel region and the secretion unit peptide is still able to function effectively.

Hence by “variant” the present invention also encompasses where the amino acid sequence of the surface loops is altered or removed. Preferably the deletion is of loop 3 (amino acids 1129 to 1136 according to the numbering used in Pet AT SEQ ID NO:1). As way of example, SEQ ID NO: 32 as provided below provides the amino acid sequence of a Pet At secretion peptide in which loop 3 has been deleted.

The “secretion unit peptide” encoded by the nucleic acid sequence within the bacterial expression construct of the first aspect of the invention comprises less than 300 amino acids of the C-terminus of a SPATE-class bacterial autotransporter polypeptide.

Accordingly, the bacterial construct of the present invention does not include nucleic acid sequence encoding a ‘full length’ SPATE-class AT polypeptide.

As stated above, an advantage of the present invention is that the inventors have determined the minimum amino acid sequence of SPATE-class AT polypeptides which can be effectively harnessed to secrete a protein of interest from the bacterial cell and mediate its release into the culture medium. This is advantageous since it is desirable to have a small a region of amino acids added the recombinant protein of interest to effect secretion.

By “less than 300” amino acids, we include where the nucleic acid sequence encodes a secretion unit peptide of 295, 290, 289, 288, 287, 286, 285, 284, 283, 282, 282, 281, 280, 279, 278, 277, 276, 275, 270 or less amino acids. Preferably the secretion unit peptide has 286 amino acids. In some embodiments of the present invention, one or more of the surface loop regions of the β-barrel region may have been removed. Where ‘loop 3’ has been removed, in such embodiments the nucleic acid sequence encodes a secretion unit peptide of 278 amino acids.

In an embodiment, the nucleic acid sequence encodes a secretion unit peptide of less than 298, less than 295, less than 290, less than 285, less than 280, less than 270, less than 260, less than 250, less than 240, less than 230 or less than 200 amino acids.

In an embodiment, the nucleic acid sequence encodes a secretion unit peptide of at least 175, at least 200, at least 220, at least 230, at least 240, at least 250, at least 260, at least 270, at least 275, at least 280 or at least 285 amino acids.

In an embodiment, the nucleic acid sequence encodes a secretion unit peptide of 286 or 294 amino acids.

A preferred embodiment of the present invention is wherein the nucleic acid sequence encoding the secretion unit peptide comprises the nucleic acid sequence provided in any one of SEQ ID NOs 3, 6, 9, 12, 15, 18, 21, 24, 27 or 28 or a variant thereof, wherein the variant encodes a secretion unit peptide capable of mediating the extracellular secretion of a peptide from the periplasm.

In this context when referring to nucleic acid molecules, by “variant” we include where the nucleic acid sequence encodes a secretion unit peptide having those variations discussed above. Also, it can be appreciated that the nucleic acid sequence of SEQ ID NOs 3, 6, 9, 12, 15, 18, 21, 24 or 27 can also be altered without changing the amino acid sequence of the encoded secretion unit peptide. For example, the nucleic acid sequence can be ‘codon optimised’ for expression in E. coli, a routine modification well known to the skilled person. Further changes can be made so as to remove multiple restriction enzyme sites to facilitate subsequent genetic manipulations using the expression construct.

As way of example, we provide in SEQ ID NO:28 nucleic acid sequence encoding a secretion unit peptide from the Pet autotransporter, where the codons have been optimised for expression in E. coli.

A preferred embodiment of the first aspect of the invention is wherein expression construct comprises nucleic acid sequence encoding a secretion unit consisting of less than 300 amino acids of the C-terminus of a SPATE-class bacterial autotransporter polypeptide, said secretion unit peptide comprising: (i) the α-helix; (ii) linker; and (iii) β-barrel region of the β-domain of the autotransporter polypeptide.

Preferably the nucleic acid sequence encodes a secretion unit consisting of the amino acid sequence provided in any one of SEQ ID NOs 2, 5, 8, 11, 14, 17, 20, 23 or 26, or a variant thereof, wherein said variant mediates the extracellular secretion of a peptide from the periplasm. Preferably the nucleic acid sequence encodes a secretion unit consisting of the amino acid sequence provided in SEQ ID NO: 2.

The description of the present application provides detailed information on the amino acid sequence of representative secretion unit peptides, as well as nucleic acid sequence encoding such peptides.

The preparation of bacterial expression constructs of the present invention can therefore be readily achieved using information in the art without any inventive requirement. In particular, we provide herein details of representative bacterial expression cassettes (e.g. the generic pASK-IBA33plus expression vector and pET22b vector) and detailed information on the nucleic acid sequences encoding secretion unit peptides.

It can therefore be appreciated that commonly used laboratory techniques for manipulating recombinant nucleic acid molecules can be used to derive the claimed bacterial expression construct.

A variety of methods have been developed to operably link polynucleotides, especially DNA, to vectors for example via complementary cohesive termini. Suitable methods are described in Sambrook et al (1989) Molecular Cloning, A Laboratory Manual, Cold Spring Harbor Laboratory, Cold Spring Harbor, N.Y.

A desirable way to modify the DNA encoding a polypeptide of the invention is to use the polymerase chain reaction. This method may be used for introducing the DNA into a suitable vector, for example by engineering in suitable restriction sites, or it may be used to modify the DNA in other useful ways as is known in the art. Hence nucleic acid sequence encoding a secretion unit peptide comprising less than 300 amino acids of the C-terminus of a SPATE-class bacterial autotransporter polypeptide can be readily prepared according to the information provided herein and located in a bacterial expression construct.

A discussion on the preparation of examples of bacterial expression constructs according to the first aspect of the invention is provided herein.

A further embodiment of the first aspect of the invention is wherein the bacterial expression construct further comprises a multiple cloning site located 5′ to the nucleic acid sequence encoding the N-terminal amino acid of the secretion unit.

The term ‘multiple cloning site’ is well known in the art. Also called a ‘polylinker’, it is a short segment of DNA which contains many restriction sites hence facilitating the insertion of nucleic acid sequences in the expression construct using procedures involving molecular cloning or subcloning.

A further embodiment of the first aspect of the invention is wherein the bacterial expression construct further comprises a nucleic acid sequence encoding a bacterial inner membrane signal peptide.

As briefly discussed above, the Pet AT polypeptide carries an N-terminal signal sequence required for protein transport through inner membrane in a SecB-dependent manner. An example of a N-terminal signal sequence used in Pet is provided in SEQ ID NO:29 at the end of the description and corresponds to the first 52 amino acids from the Pet sequence shown in SEQ ID NO:1.

An example of a nucleic acid sequence encoding SEQ ID NO:29 is provided in SEQ ID NO:30 at the end of the description.

As discussed above, the nucleic acid sequence encoding the Pet autotransporter has been ‘codon optimised’ for expression in E. coli. We provide in SEQ ID NO: 31 at the end of the description nucleic acid sequence encoding the N-terminal signal sequence used in Pet, ‘codon optimised’ as discussed.

However, as can be appreciated further bacterial inner membrane signal peptides can be readily used, and hence further nucleic acid sequences encoding a bacterial inner membrane signal peptide as stated in this embodiment of the invention can easily be identified by the skilled person. Indeed, the present inventors have demonstrated that multiple different signal sequences that target different inner membrane translocation pathways, can be used to direct Pet to the periplasm (Leyton et al (2010) FEMS Microbiol Letts, 311, 133-139).

An embodiment of the invention is wherein the expression construct has the following structure: (i) nucleic acid encoding a bacterial inner membrane signal peptide, operatively linked at the 3′ with (ii) a multiple cloning site, operatively linked at the 3′ with (iii) nucleic acid encoding the secretion unit.

In such an arrangement, when a gene encoding a protein of interest is placed in the multiple cloning site such that the protein of interest is operatively linked with the secretion unit peptide, upon introduction to a suitable host cell the bacterial expression construct will encode a heterologous polypeptide molecule having: (i) an N-terminal bacterial inner membrane signal peptide; (ii) a protein of interest; (iii) a C-terminal secretion unit peptide. Such a heterologous polypeptide molecule will be exported to the periplasm, where the inner membrane signal peptide will be cleaved, and the protein of interest/secretion unit peptide fusion will be translocated across the outer membrane. The secretion unit peptide will then be cleaved, and the protein of interest released in to the extracellular milieu.

An embodiment of the invention is wherein the expression construct further comprises a second nucleic acid sequence encoding a protein of interest located at the multiple cloning site, the second nucleic acid arranged such that the protein of interest is operatively linked with the secretion unit peptide.

A further embodiment of the invention is wherein the expression construct encodes a recombinant polypeptide having the following structure: (i) a bacterial inner membrane signal peptide, operatively linked at the C-terminus with (ii) a protein of interest, operatively linked at the C-terminus with (iii) the secretion unit peptide.

As explained further below in Example 1, the inventors have prepared specific embodiment of the expression constructs according to the first aspect of the invention.

pASK-ESAT6-PetΔ*20 is one such expression construct. It was prepared as follows. A nucleic acid sequence encoding the Pet AT polypeptide was inserted into the pASK-IBA33plus bacterial expression plasmid (purchased from IBA). Nucleic acid sequence encoding the ESAT6 polypeptide (used as an example of a ‘protein of interest’) was then cloned in to the BglII and PstI sites in the Pet nucleic acid sequence (see FIG. 2) to generate pASK-ESAT6-Pet-BP. The region from the PstI site to the codon encoding amino acid 1009 of Pet polypeptide was then deleted, providing the pASK-ESAT6-PetΔ*20 expression construct. This expression construct therefore encodes a fusion protein having: (i) a bacterial inner membrane signal peptide (in this case the native Pet signal peptide), operatively linked at the C-terminus with (ii) a protein of interest (in this case ESAT6), operatively linked at the C-terminus with (iii) the secretion unit peptide (in this case amino acids 1010-1295 of Pet).

pASK-ESAT6-PicΔ*20 is a further such expression construct. It was prepared by replacing the nucleic acid sequence encoding amino acids 1010-1295 of Pet in the pASK-ESAT6-PetΔ*20 with nucleic acid sequence encoding the equivalent fragment from the Pic nucleic acid sequence. This expression construct therefore encodes a fusion protein having: (i) a bacterial inner membrane signal peptide (in this case the native Pet signal peptide), operatively linked at the C-terminus with (ii) a protein of interest (in this case ESAT6), operatively linked at the C-terminus with (iii) the secretion unit peptide (in this case amino acids 1087 to 1372 of Pic).

It can be appreciated that pASK-ESAT6-PetΔ*20 and pASK-ESAT6-PicΔ*20 can be readily altered to encode a different ‘protein of interest’ by simply replacing the nucleic acid encoding the ESAT6 polypeptide.

In addition to the particular components of the bacterial expression construct provided above, further nucleic acid molecules can be included. For example, nucleic acid sequences encoding amino acid tags useful for facilitating isolation of the protein of interest from the extracellular milieu can be included, such as commonly used His-tag system, as well known to the skilled person.

The bacterial expression construct of the first aspect of the invention should be introduced into a suitable host cell to mediate expression of the recombinant protein. There are many standard laboratory techniques that can be adopted by the skilled person to introduce expression constructs to host cells. Generally, not all of the hosts will be transformed by the vector and it will therefore be necessary to select for transformed host cells. One selection technique involves incorporating into an expression construct containing any necessary control elements a DNA sequence marker that codes for a selectable trait in the transformed cell. These markers include dihydrofolate reductase, G418 or neomycin resistance for eukaryotic cell culture, and tetracycline, kanamycin or ampicillin resistance genes for culturing in E. coli and other bacteria. The selectable markers could also be those which complement auxotrophisms in the host. Alternatively, the gene for such a selectable trait can be on another vector, which is used to co-transform the desired host cell.

The host cell should be a Gram-negative bacterial species, preferably E. coli, Shigella, Salmonella, Yersinia or Klebsiella.

As is well known in the field, mutated derivatives of such Gram-negative bacterial species have been prepared that improve the quality and/or quantity of the amount of protein produced. They can therefore be used as host cells to mediate expression of the recombinant protein from the expression construct of this aspect of the invention.

In particular, the following bacterial strains are particularly useful for the aspects of the invention:

E. coli TOP10 F-mcrk A Δ(mrr-hsdRMS-mcrBC) φ80lacZΔM15 ΔlacX74 recA1 araD139 Δ(araleu) 7697 ga/U ga/K rpsL enc/A1 nupG (available from Invitrogen)
E. coli BL21 (DE3) fhuA2 [Ion] ompT gal (λ DE3) [dcm] ΔhsdS λ DE3=λ sBamHIo ΔEcoRI-B int::(lacI::PlacUV5::T7 gene1) i21 Δnin5 (available from New England Biolabs)
E. coli JM109 endA1, recA1, gyrA96, thi, hsdR17 (rk−, mk+), relA1, supE44, D(lac-proAB), [F¢, traD36, proAB, laqIqZDM15] (available from Promega

A second aspect of the invention provides a host cell comprising a bacterial expression construct according to the first aspect of the invention. Preferably the host cell is a Gram-negative bacterium.

The invention also relates to a host cell expressing one or more fusion proteins wherein said fusion protein comprises the secretion unit peptide as defined herein and a protein of interest. All of the particular embodiments of the bacterial expression construct according to the first aspect of the invention can be utilised in the host cell of this aspect of the invention. Hence the preceding discussion on that aspect of the invention also applies to the second aspect of the invention.

Methods of preparing a bacterial expression construct according to the first aspect of the invention as provided above, as are methods of preparing a host cell comprising that bacterial expression construct.

Preferably the host cell is a Gram-negative bacterial species, preferably E. coli, Shigella, Salmonella, Yersinia or Klebsiella. Preferably the host cell is a bacterial strain, for example:

E. coli TOP10 F-mcrA Δ(mrr-hsdRMS-mcrBC) φ80lacZΔM15 ΔlacX74 recA1 araD139 Δ(araleu) 7697 ga/U ga/K rpsL endA1 nupG (available from Invitrogen)
E. coli BL21 (DE3) fhuA2 [Ion] ompT gal (λ DE3) [dcm] ΔhsdS λ DE3=λsBamHIo ΔEcoRI-B int::(lacI::PlacUV5::T7 gene1) i21 Δnin5 (available from New England Biolabs)
E. coli JM109 endA1, recA1, gyrA96, thi, hsdR17 (rk−, mk+), relA1, supE44, D(lac-proAB), [F¢, traD36, proAB, laqIqZDM15] (available from Promega)

A third aspect of the invention provides a recombinant peptide comprising a secretion unit peptide comprising less than 300 amino acids of the C-terminus of a SPATE-class bacterial autotransporter polypeptide, said secretion unit peptide comprising: (i) the α-helix; (ii) linker; and (iii) β-barrel region of the β-domain of the autotransporter polypeptide.

For the avoidance of doubt, the particular embodiments of the secretion unit peptide defined above in relation to the first aspect of the invention apply to the third aspect of the invention. Hence where relevant the preceding discussion on that aspect of the invention also applies to the third aspect of the invention.

Examples of amino acid sequences for the secretion unit peptide of this aspect of the invention are provided in relation to the first aspect of the invention. In particular, embodiments of third aspect of the invention include where the secretion unit peptide is derivable from a SPATE-class bacterial autotransporter polypeptide selected from the following: Pet, Sat, EspP, SigA, EspC, Tsh, SepA, and Pic. Preferably the secretion unit peptide is derivable from one or more SPATE-class bacterial autotransporter polypeptides selected from the following: PET_ECO44, SAT_CFT073, ESPP_ECO57, SIGA_SHIFL, ESPC_ECO27, TSH_{—}E. coli, SEPA_EC536, PIC_ECO44, SEPA_SHIFL.

In particular, the secretion unit peptide of the third aspect of the invention comprises the amino acid sequence provided in any one of SEQ ID NOs 2, 5, 8, 11, 14, 17, 20, 23 or 26 or a variant thereof, wherein the variant is capable of mediating the extracellular secretion of a peptide from the periplasm.

It is preferred that the secretion unit peptide consists of less than 300 amino acids of the C-terminus of a SPATE-class bacterial autotransporter polypeptide, said secretion unit peptide comprising: (i) the α-helix; (ii) linker; and (iii) β-barrel region of the β-domain of the autotransporter polypeptide. Preferably the secretion unit peptide consists of the amino acid sequence provided in any one of any one of SEQ ID NOs 2, 5, 8, 11, 14, 17, 20, 23 or 26 or a variant thereof, wherein the variant is capable of mediating the extracellular secretion of a peptide from the periplasm. More preferably the secretion unit peptide consists of the amino acid sequence provided in SEQ ID NO: 2.

Examples of nucleic acid sequences encoding a secretion unit peptide according to the third aspect of the invention as provided above, for example the nucleic acid sequence encoding the secretion unit peptide comprises the nucleic acid sequence provided in any one of SEQ ID NOs 3, 6, 9, 12, 15, 18, 21, 24, 27 or 28 or a variant thereof, wherein the variant encodes a secretion unit peptide capable of mediating the extracellular secretion of a peptide from the periplasm.

As can be appreciated, the secretion unit peptide according to the third aspect of the invention can be prepared using the information presented herein. In particular, the secretion unit peptide can be prepared de novo using routine peptide synthesis techniques, or the secretion unit peptide can be prepared by expressing a nucleic acid sequence encoding a secretion unit peptide, as provided herein, in an appropriate host cell, and isolating the expressed peptide from that cell using well known and routine laboratory methods.

A fourth aspect of the invention provides a nucleic acid molecule comprising a sequence encoding the recombinant peptide of the third aspect of the invention.

For the avoidance of doubt, the particular embodiments of the nucleic acid molecules defined above in relation to the first aspect of the invention apply to the fourth aspect of the invention. Hence where relevant the preceding discussion on that aspect of the invention also applies to the fourth aspect of the invention.

Examples of nucleic acid sequences encoding a secretion unit peptide according to the third aspect of the invention as provided above, for example the nucleic acid sequence encoding the secretion unit peptide comprises the nucleic acid sequence provided in any one of SEQ ID NOs 3, 6, 9, 12, 15, 18, 21, 24, 27 or 28 or a variant thereof, wherein the variant encodes a secretion unit peptide capable of mediating the extracellular secretion of a peptide from the periplasm.

As can be appreciated, the nucleic acid sequence according to the fourth aspect of the invention can be prepared using the information presented herein. In particular, the nucleic acid can be prepared de novo using routine nucleic acid synthesis techniques, or isolated from a larger polynucleotide sequence encoding a SPATE-class bacterial autotransporter polypeptide or homologous protein.

A fifth aspect of the invention provides a recombinant fusion protein comprising a peptide according to the third aspect of the invention fused with a protein of interest.

For the avoidance of doubt, the particular embodiments of the peptide according to the third aspect of the invention are defined above and are relevant to the fifth aspect of the invention. Hence where relevant the preceding discussion on that aspect of the invention also applies to the fifth aspect of the invention.

As can be appreciated, the recombinant fusion protein according to the fifth aspect of the invention can be prepared using the information presented herein. In particular, a gene encoding a protein of interest can be located in a bacterial expression construct according to the first aspect of the invention. Upon introduction to a suitable host cell, the bacterial expression construct will encode a recombinant fusion protein molecule of the fifth aspect of the invention.

A sixth aspect of the invention provides a method of secreting a polypeptide from a periplasm, the method comprising fusing a secretion unit peptide comprising less than 300 amino acids of the C-terminus of a SPATE-class bacterial autotransporter polypeptide, said secretion unit peptide comprising: (i) the α-helix; (ii) linker; and (iii) β-barrel region of the β-domain of the autotransporter polypeptide, to the C-terminus of the polypeptide.

Preferably the method further comprises arranging for the fusion protein to be expressed in a suitable host cell, as discussed above, during which the secretion unit peptide will direct secretion of the polypeptide from the periplasm.

A seventh aspect of the invention provides the use of a secretion unit peptide comprising less than 300 amino acids of the C-terminus of a SPATE-class bacterial autotransporter polypeptide, said secretion unit peptide comprising: (i) the α-helix; (ii) linker; and (iii) β-barrel region of the β-domain of the autotransporter polypeptide for secretion of a polypeptide from a bacterial periplasm.

Particular embodiments of the sixth and seventh aspects of the invention are wherein the secretion unit peptide is as defined in relation to the first and third aspects of the invention.

By “polypeptide” we include “protein of interest” as described above in relation to earlier aspects of the invention.

An eighth aspect of the invention provides a method of preparing a recombinant polypeptide, the method comprising culturing the host cell of the second aspect of the invention in a culture medium so as to obtain the expression and secretion of the recombinant polypeptide into the culture medium.

For the avoidance of doubt, by “recombinant polypeptide” we mean the “protein of interest” as described above in relation to the first aspect of the invention.

The method of the eighth aspect of the invention comprises culturing the host cell described above for a sufficient time and under appropriate conditions in a culture medium so as to obtain expression of the recombinant polypeptide from the bacterial expression construct.

As stated above, the expression construct is used for the efficient expression and secretion of a protein of interest from a bacterial cell to the extracellular milieu. In use, a gene encoding a protein of interest is cloned in to the expression construct such that the gene is operatively linked with the nucleic acid sequence encoding a secretion unit peptide. Upon introduction of the bacterial expression construct into an appropriate host cell, the protein of interest and the secretion unit peptide are formed as a single polypeptide molecule. The heterologous fusion polypeptide molecule will be exported to the periplasm, where the inner membrane signal peptide will be cleaved, and the protein of interest/secretion unit peptide fusion will be translocated across the outer membrane. The secretion unit peptide will then be cleaved, and the protein of interest, i.e. the recombinant polypeptide, released in to the extracellular milieu.

The recombinant polypeptide can be readily isolated from the culture medium using standard techniques known in the art including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography.

A further embodiment of the eighth aspect of the invention comprises (i) preparing a bacterial expression construct of the first aspect of the invention, comprising a gene encoding the protein of interest; (ii) introducing the bacterial expression construct into an appropriate host bacterial cell; (iii) culturing the host cell in conditions to promote the expression and secretion of the protein of interest into the culture medium; (iv) isolating the protein of interest from the culture medium.

Methods of culturing bacterial cells are well known in the art. Examples of methods and materials which can be used in the eighth aspect of the invention are provided in the accompanying examples.

A ninth aspect of the invention provides a kit of parts comprising: (i) the expression construct as defined above; and (ii) a manual of operation.

The manual of operation can include information concerning, for example, the restriction enzyme map of the expression construct; the nucleic acid sequence of expression construct; how to introduce a gene encoding a protein of interest in to the expression construct; optional conditions for expression of the protein of interest in a suitable host cell, and other such information as appropriate.

The kit of parts can further comprise further components, for example, transformation competent host cells for expression of the expression construct; enzymes that can be used to prepare an expression construct harbouring a gene encoding a protein of interest, such as typical restriction enzymes; enzymes that can be used to amplify the copy number of a gene encoding a protein of interest, such as DNA polymerase, preferably Taq, Pfu, or further well-known thermostable DNA polymerases; ‘test control’ agents such as control plasmid inserts. The kit may also comprise reagents useful for the recovery of the protein of interest from the cell supernatant, such as protein purification columns or resins.

The invention is now described by reference to the following, non-limiting, figures and examples.

### FIGURE LEGENDS

FIG. 1. Schematic of Autotransporter secretion.

FIG. 2. Construction of heterologous protein fusions with Pet. Heterologous protein insertions in the Pet passenger domain are shown by boxes marked ‘HP’ or with the name of the protein; the latter are also listed on the right. Abbreviations BC, BB and BP on the left refer to the type of protein fusion generated by insertion of foreign DNA into the pet gene between the restriction sites BglII-ClaI, BglII-BstBI or BglII-PstI, respectively. The co-ordinates above the figure are given for the amino acids derived from the pet gene sequence. The arrow at position 1018 denotes the cleavage site in the α-helix that effects release of the passenger domain into the culture medium. Modification of this site results in surface display of molecules. The abbreviations SS, AC, HSF, α and L denote the positions of the signal sequence, autochaperone domain, hydrophobic secretion facilitator, α-helix and linker, respectively.

FIG. 3. Identification of the minimal AT module permitting secretion of heterologous proteins to the culture supernatant. Schematic of ESAT6-Pet-BP protein fusion and truncations created to determine the minimal C-terminal Pet fragment capable of ESAT-6 secretion, in accordance with an embodiment of the present invention, is shown. Abbreviations are the same as in FIG. 2. Length of the C-terminal Pet fragment present in the truncation mutants is shown in brackets.

### SUMMARY

In this study the inventors replaced Pet passenger domain with a number of heterologous proteins such as ESAT-6, Ag85B, mCherry, pertactin (Prn), LatA, SapA, Pmp17, YapA, and BMAA1263 and have shown secretion of resulting protein chimeras into culture supernatants. These constructs contained Pet signal sequence at their N-termini and the Pet C-terminus of varying lengths. Notably, all N-terminal Pet truncations were able to promote secretion of the N-terminally fused recombinant protein partners into culture medium. The smallest secretion-proficient truncations, Pet817-1295 and Pet889-1295, lacked most (or all) of the Pet (3-helical stem structure but included complete autochaperone (AC) domain. In native Pet protein, the AC domain comprises last 100 amino acids of the passenger domain followed by 19 amino acid-long hydrophobic secretion facilitator (HSF) domain which separates the Pet passenger from the translocation domain (α-helix and the β-barrel). In the art the AC domain is thought to be essential for folding and secretion of native Pet protein but its role in, or requirement for, the heterologous protein secretion is unknown.

Using ESAT-6 as a model protein, the inventors endeavoured to identify the smallest part of Pet C-terminus that can promote ESAT-6 secretion into culture medium. For this, the inventors constructed a series of ESAT-6-Pet fusions in which the length of Pet C-terminus was gradually reduced from Pet817-1295 to Pet1010-1295 through sequential (nested) deletions within the AC-HSF, and of the entire AC-HSF domain region. Surprisingly, analysis of ESAT-6 secretion showed that Pet truncations lacking whole of the AC domain (Pet988-1295) efficiently secreted ESAT-6 into culture supernatants. Surprisingly removal of the HSF domain (Pet1010-1295) still allowed ESAT-6 secretion. This data shows that the AC domain and HSF domain are not essential for heterologous protein secretion. The C-terminal Pet fragment, Pet 1010-1295, was therefore identified as a minimal part of Pet that can mediate translocation and release of heterologous proteins into extracellular environment in E. coli.

### INTRODUCTION, RESULTS AND DISCUSSION

To be an effective system for recombinant protein production an Autotransporter system needs to undergo autoprocessing, where the recombinant target protein is released into the extracellular milieu.

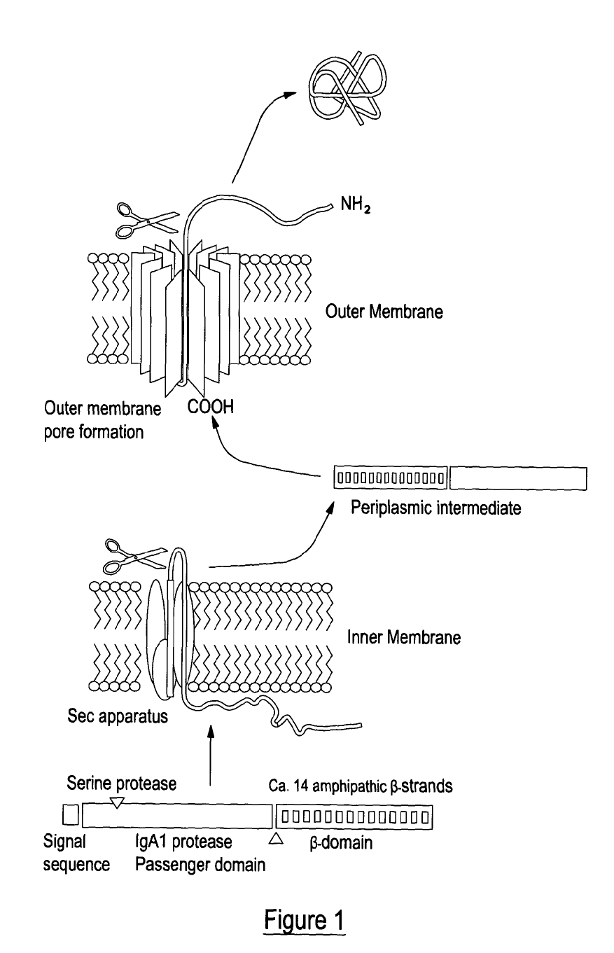

Pet is an enterotoxin secreted by enteroaggregative E. coli and is a member of autotransporter protein family (type Va secretion system); it belongs to a subgroup of the Autotransporters termed the SPATEs (serine protease autotransporters of the Enterobacteriaceae). Pet carries an N-terminal signal sequence required for protein transport through inner membrane in a SecB-dependent manner, a passenger domain where the effector function (serine protease) is encoded, and a C-terminal β-barrel that mediates passenger domain translocation to the cell surface (FIG. 1). Unlike many other autotransporters that remain attached to its β-barrel or associated with the outer membrane, the Pet passenger domain, which encodes the toxin function, is cleaved off and secreted into extracellular environment. Due to this property, together with the apparent simplicity of the autotransporter secretion mechanism, Pet can be exploited for secretion of soluble recombinant proteins into the culture medium. However, the amino acid sequences required for effective release into the culture milieu have not been defined.

Here the inventors demonstrate that Pet, and other SPATE-class AT proteins, can be utilised for release of recombinant proteins into the culture medium, where it accumulates as a soluble protein. In accordance with an embodiment of the present invention, they demonstrate the minimal amino acid sequences required to allow secretion to occur. They also demonstrate which regions of Pet can be manipulated while allowing secretion to be maintained.

### Fusions to the Pet Passenger Domain can be Secreted to the External Milieu.

To be effective for recombinant protein production Pet must be efficient at secretion of non-native proteins. To test this, the inventors made series of fusions to the Pet passenger domain. These fusions utilised a pet gene construct that was synthesised de novo by GenScript and cloned into the generic pASK-IBA33plus expression vector; expression was induced from the tet promoter/operator in E. coli TOP10. The pet gene sequence was codon optimised using E. coli codon usage and cleared of multiple restriction sites while unique restriction sites were engineered in this sequence to facilitate genetic manipulations. These procedures did not change native Pet protein translation. Thus the pet cassette has been made suitable for in-frame insertions of genes encoding recombinant proteins of interest and easy engineering of such features as affinity purification tags, protease cleavage sites and for convenient site mutagenesis.

To test the ability of Pet to mediate secretion of heterologous proteins into the culture medium we chose proteins with distinctive size, structural and functional signatures. These included the secreted portions of (1) Pertactin (44 kDa) from Bordetella pertussis, a component of the acellular whooping cough vaccine, (2) YapA (105 kDa), a surface protein from Yersinia pestis, (3) Pmp17 (40 kDa), a polymorphic surface protein from Chlamydophila abortus, (4) SapA (60 kDa), a putative surface protein from Salmonella enterica serovar Typhimurium, (5) the red fluorescent protein mCherry (27 kDa), a derivative of Discosoma sp DsRed, (6) the predicted secreted esterase Ag85B (35 kDa), a putative Mycobacterium tuberculosis vaccine candidate, (7) ESAT-6 (10 kDa) the major diagnostic marker from M. tuberculosis, (8) LatA (44 kDa), a predicted surface protein from Lawsonia intracellularis, and (9) BMAA1263 (71 kDa), a putative surface protein from Burkholderia mallei.

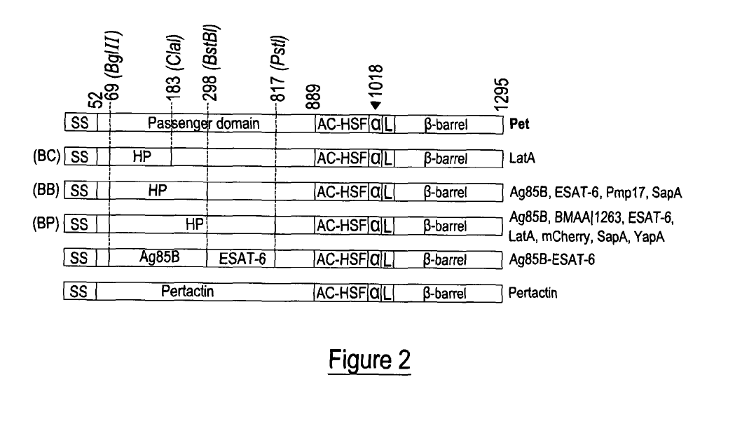

DNA encoding the heterologous proteins was synthesized de novo after codon optimization for expression in E. coli and part of the pet gene was replaced in-frame with the heterologous genes to give rise to fusion proteins as shown in FIG. 2. Insertion of the nucleotide sequence encoding LatA between BglII and ClaI restriction sites in pet resulted in deletion of the nucleotide sequence encoding 114 amino acids of Pet that covered much of domain 1, which is globular in structure and confers serine protease activity. Secretion of the resulting LatA-Pet-BC protein fusion into culture medium in E. coli TOP10 was confirmed by Western blotting with anti-Pet antibodies. Insertions of heterologous DNA between BglII and BstBI (BB fusions) or BglII and PstI (BP fusions) pet restriction sites resulted in removal of some or most of the Pet passenger β-helix (FIG. 2). The resulting protein fusions included the N-terminal Pet signal sequence, and either Pet 298-1295 (BB fusions) fragment or Pet 817-1295 (BP fusions) fragment at the C-terminus, both containing the predicted Pet AC-HSF domain. Secretion of Pet fusions with Ag85B, ESAT-6, Pmp17, SapA, BMAA1263, mCherry, LatA and YapA proteins into culture supernatants in E. coli TOP10 was demonstrated. To authenticate the identities of some of the secreted protein fusions, bands were excised from polyacrylamide gels and subjected to mass spectrometry; the appropriate protein was confirmed in each case (Table 1). As additional marker of secretion of the chimeric proteins, the cleaved Pet β-barrel accumulated in the outer membrane (OM) at levels similar to that for wild-type Pet.

Fusions to the Pet Passenger Domain can be Secreted to the External Milieu When Expressed from Different Plasmids.

To ensure the Pet system was capable of secreting recombinant proteins to the culture medium when cloned in a separate expression plasmid a further construct was made. The construct designated pET-prn-pet was synthesised such that the pertactin-pet chimeric sequence (encoding Pertactin-Pet protein fusion) was de novo synthesised (GenArt) and cloned into a pET22b vector under the control of T7lac promoter. In this construct the secreted domain of Bordetella pertussis pertactin protein (amino acids 35-471) is flanked by the 52 amino acid Pet signal sequence at the N-terminus and the 406 amino acid Pet translocation domain (Pet889-1295) at the C-terminus (FIG. 2). Secretion of Pertactin-Pet fusion protein into culture supernatant in E. coli BL21*(DE3) was confirmed by Western blotting with anti-Pet antibodies and accumulation of cleaved Pet β-barrel in the OM was shown.

### Pet can Mediate Secretion of Proteins Containing Cysteine Residues

Proteins targeted for secretion to the exterior of the cell often traverse the periplasm. The periplasm is a highly oxidising environment which promotes disulphide bond formation between cysteine amino acids; the enzyme DsbA catalyses this reaction. To test whether Pet could secrete proteins containing cysteine amino acids to the external culture medium, a Pmp17-Pet-BB fusion (FIG. 2) that contains 7 cysteine residues was produced in a wild-type E. coli K-12 (E. coli TOP10) background and an equivalent dsbA mutant. The Pmp17-Pet-BB secreted by dsbA mutant accumulated in the culture medium at levels similar to wild type Pet while no full-length fusion could be detected in wild type E. coli TOP10. These results are consistent with the observations made for other ATs that disulphide bond formation and partial folding of native or heterologous polypeptides hinder their secretion. Thus, the Pet protein production system is capable of efficiently secreting recombinant proteins that contain multiple cysteine residues when expressed in a mutant strain which does not promote disulphide bond formation.

### Pet can Secrete Multivalent Proteins

It may be desirable at times to reduce cost of recombinant protein production by expressing separate distinct proteins as polyvalent molecules. To test whether the Pet secretion system was effective at producing multivalent chimeras, the pASK-Ag85B-ESAT6 construct was made in which Ag85B and ESAT-6 protein-encoding DNA was inserted in tandem in the pet gene between BglII-BstBI-PstI sites (FIG. 2). The resulting fusion protein Ag85B-ESAT6-Pet was produced in E. coli TOP10 and its secretion into culture supernatant was confirmed by Western blotting with anti-ESAT6 and anti-Pet antibodies; the presence of cleaved Pet β-barrel in the OM was also shown.

Pet can Mediate Secretion of Proteins with Purification Tags.

Although secretion of heterologous proteins to the culture medium overcomes many of the barriers associated with purification of proteins from the intact cell a number of process impurities may still remain. The target protein can be removed from these process impurities in a variety of manners including by use of purification tags. Thus we assessed if the Pet system was capable of producing proteins with some of the common sequence tags, including. purification tags. Amino acid sequences encoding a His₆-tag or a FLAG-tag were engineered, after signal sequence cleavage site, into Pet, its deletion derivatives, Pmp17-Pet-BB and SapA-Pet-BP using standard molecular biology procedures. Similarly, FS-ESAT6-Pet-BP variant was engineered to contain a 25 amino acid long fusogenic sequence tag at the N-terminus. In all cases the tagged proteins accumulated in the culture supernatants (and corresponding cleaved β-barrels in the OM) demonstrating that purification tags can be attached to proteins destined for secretion into the extracellular milieu.

Fusion Proteins Secreted by Pet into Culture Medium Show Correct Folding

To be useful as a method of recombinant protein production, the AT system must be able to secrete soluble, folded and functional proteins into the culture medium. To test if the chimeric proteins were natively folded after secretion, His₆-tagged derivatives of YapA-Pet-BP, mCherry-Pet-BP and wild type Pet were harvested from the culture supernatant fractions and subjected to analysis by circular dichroism (CD). YapA is predicted to possess a mixed α-helical/β-strand conformation and mCherry is known to adopt a β-barrel conformation. CD spectra of YapA showed minima at 222 nm and 208 nm and maxima at 195 nm indicative of a folded protein with mixed α-helical/β-strand content. Consistent with their natively folded β-strand conformations, CD spectra for Pet and mCherry showed minima at 218 nm and maxima at 195 nm. Additionally, mCherry purified from the culture supernatant fraction showed red fluorescence indicating a folded protein with functional activity.

Yields of Heterologous Protein Fusions with Pet in Culture Supernatants

Effective utilisation of the AT module for generalised protein secretion necessitates achieving yields of target proteins at industrial scale and at concentrations competitive with alternative technologies. Secreted yields of Pertactin and ESAT-6 from Pet fusion constructs in shake flasks were calculated. For ESAT6-Pet variants concentrations of 5.4 mg/l were achieved after expression in E. coli BL21*. For Pertactin yields of 1 mg/l were achieved after expression in E. coli BL21* cultures. Importantly, these are yields for small-scale non-optimised conditions and they are consistent with levels achieved for other E. coli protein secretion systems; higher protein yields could be generated in a controlled, optimised fermentation process.

### Pet-Driven Heterologous Protein Secretion is not Due to Cell Lysis or Periplasmic Leakage

To demonstrate that the presence of heterologous proteins in the culture medium was due to secretion rather than leakage from the periplasm, we examined the cellular location of mCherry and ESAT-6. For this we used the non cleaved Pet derivative, Pet*, that contains N1018G and D1115G substitutions. These mutations disrupt the interdomain cleavage site such that the passenger domain is completely translocated to the cell surface but remains covalently attached to the β-domain. These mutations were introduced into mCherry-Pet-BP and ESAT6-Pet-BB to create mCherry* and ESAT6*. In each case no passenger domain accumulated in the culture medium and full length protein species were detected in the OM. Immunofluorescence and flow cytometry studies of bacteria expressing Pet*, mCherry* and ESAT6* with specific antibodies revealed surface localisation of passenger domains whereas with the native cleavage site there was negligible staining. These experiments demonstrated that the heterologous fusions were expressed and actively translocated to the cell surface before cleavage. Crucially, probing with antibodies directed at the periplasmic protein BamD revealed labelling was not due to egress of antibodies into the bacterial cell since cells did not label unless permeabilised by chemical treatment; hence secretion occurred without major loss of membrane integrity.

To ensure proteins were not released into the culture medium by cell lysis upon induction of expression, staining with propidium iodide (PI) and Bis-(1,3-dibutylbarbituric acid) trimethine oxonol (BOX) was used to assess cell viability and the integrity of the cell envelope of bacteria secreting heterologous fusions. Flow cytometry analyses of cultures expressing ESAT6-Pet-BB, ESAT6-Pet-BP and Pet proteins revealed that the majority of cells remain healthy and alive during protein secretion with only negligible increases in the number of BOX- or PI+BOX-positive cells after induction of protein expression compared to uninduced cultures. Finally, assays for alkaline phosphatase, a periplasmic enzyme, demonstrate no leakage of periplasmic proteins after expression of heterologous fusions. Taken together these data indicate that the presence of secreted proteins in the culture media is not due to cell lysis or periplasmic leakage, but active secretion.

Additionally, the presence of ESAT-6 and fluorescent mCherry on the bacterial cell surface of cultures expressing mCherry* and ESAT6*, indicated the Pet AT-module, lacking the cleavage site, can also be used for autodisplay of functional proteins on the bacterial cell surface.

### The Minimal Pet Construct Required for Secretion

Having established that the Pet Autotransporter system can be used for secretion of non-native proteins to the culture supernatant, the inventors determined the minimal portion of Pet that is required to achieve such secretion in order to provide a system that is useful for commercial protein expression.

The inventors used the ESAT6-Pet-BP fusion described above as a starting point (FIG. 2); this construct contains the Mycobacterial ESAT-6 protein fused to a Pet fragment corresponding to amino acids 817-1295 of native Pet. This construct contains amino acids sequences forming a portion of the β-helical stem of the passenger domain (817-888); the AC domain (amino acids 889-989), the 21 amino acid-long hydrophobic secretion facilitator (HSF) domain (990-1009), the 14 amino acid-long α-helix that spans the pore of the β-barrel and includes the cleavage site (1010-1024), a 10 amino acid linker region (1025-1033) and the β-barrel (1034-1295).

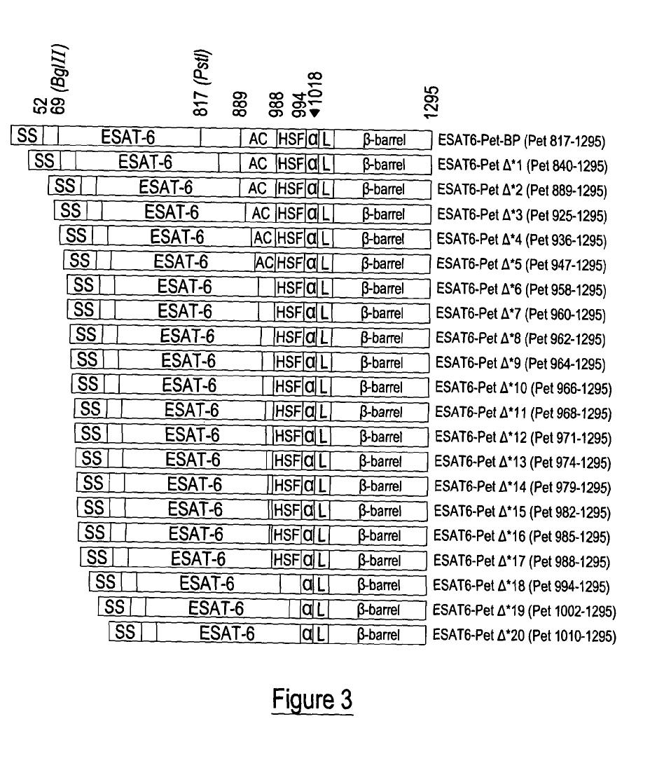

Previously, the AC domain has been implicated in secretion of AT passenger domains, where contemporaneous folding of the (3-helix and a Brownian ratchet mechanism provide the vectorial impetus for secretion. We sought to determine the precise length of the minimal functional translocation domain for Pet and establish whether the AC-domain is required for secretion of heterologous proteins to the growth medium. For this, we created 20 constructs derivative from pASK-ESAT6-Pet-BP and harbouring sequential truncations within the pet gene fragment encoding Pet817-1295 (FIG. 3).

All ESAT6-Pet fusion proteins were successfully secreted into culture medium in E. coli TOP10. It was surprisingly found that the smallest secretion-proficient Pet fragment is Pet1010-1295 in ESAT6-PetΔ*20. This fragment encodes a secretion unit peptide which comprises just 286 amino acids of the C-terminus of the Pet autotransporter polypeptide, the secretion unit containing only the predicted α-helix, linker, and the downstream β-barrel domain (ESAT6-PetΔ*20) and lacks any upstream sequences. The ESAT-6 protein secreted by this Pet derivative contains, at the C-terminus, only the 9 amino acids of the wild type Pet passenger domain α-helix that are juxtaposed with the cleavage site. The construct ESAT6-PetΔ*19, which comprises 294 amino acids of Pet (Pet 1002-1295), was also capable of supporting secretion of ESAT-6 into the culture medium.

The construct ESAT6-PetΔ*17 was also found to be capable of supporting secretion of ESAT-6 to the culture medium. This construct encodes a secretion unit peptide comprising 308 amino acids of the C-terminus of the Pet autotransporter polypeptide (Pet 988-1295), which comprises the α-helix, linker, and the downstream β-barrel domain, and additionally comprises the HSF domain.

The data presented herein therefore shows that the shortest Pet truncation (ESAT6-Pet Δ*20), in which ESAT6 was fused to the α-helix, does still support secretion. This corresponds to amino acids 1010-1295 of Pet. All other constructs (ESAT6-PetΔ*1-Δ*19) could also support secretion of ESAT-6 to the culture medium. Surprisingly, the AC domain is not required for recombinant protein secretion. Two truncations (ESAT6-PetΔ*17 and ESAT6-PetΔ*20) differed only by the 22 amino acids that encompass the HSF domain, indicating that, contrary to current opinion in the field, the HSF domain is also not required for recombinant protein secretion by Pet (FIG. 3).

Two recent reports implicated a conserved tryptophan residue (W985) in the AC domain in secretion of some SPATEs. Interestingly, ESAT6-PetΔ*17, Δ*18, Δ*19 and Δ*20 proteins lack the predicted Pet AC domain altogether but are secreted. To further test a role for W985 in secretion, this amino acid and three other conserved and juxtaposed residues (I983, L987 and G989) were mutated to alanine and lysine in the secretion-competent ESAT6-PetΔ*6. All mutated proteins were secreted into the growth medium as efficiently as the ESAT6-PetΔ*6 and retained a cleaved β-domain in the OM. These data further support the view that the AC domain is not required for secretion perse but is essential for folding of native AT polypeptides.

To confirm this finding, the inventors conducted further experiments to demonstrate efficient secretion of ESAT6-Pet fusions from Salmonella enterica SL1344. It was found that the PetΔ*6 fragment does secrete ESAT6 to the culture supernatant and the cleaved β-barrel is retained in the OM. This confirms the Pet fragments containing 286 amino acids (Pet 1010-1295) or 294 amino acids (Pet 1002-1295) are sufficient to function as a ‘secretion unit’, and moreover this ‘secretion unit’ can function in Salmonella enterica as well as E. coli.

The HSF Domain can be Manipulated without Loss of Secretion

The AC domain and the α-helical pore spanning domain are connected by a region designated the HSF. This region has a high content of hydrophobic amino acids and is predicted from the crystal structure of the Hbp passenger domain to be unstructured. To test whether the amino acid sequence of this region was essential for secretion, the inventors created several point mutations. Thus the asparagines and aspartic acid residues (N995 and D997) were mutated to alanines; the lysines residues (K1000K1001) were converted to alanines; the alanine residues (A998A999) were converted to tryptophans and glycines. None of these mutations impacted significantly on the ability of the protein to be secreted. This demonstrates that specific amino acid sequence of the HSF is not critical for secretion to be effected, and is in agreement with the data discussed above which shows that the HSF domain is not required for recombinant protein secretion (FIG. 3).

A Secretion Unit from Pic

As can be seen above, the inventors have identified a minimum region of Pet AT polypeptide which can function as a ‘secretion unit’. They then examined where an equivalent region from Pic can also function as a ‘secretion unit’.

Pic is a member of the SPATE-class bacterial autotransporter polypeptides. Pic belongs to a clade of the SPATEs that is evolutionarily distinct from that harbouring Pet. Alignment of Pet and Pic protein sequences from the beginning of the predicted AC domains shows 68% identity and 80% similarity. An example of the polypeptide sequence of Pic is provided in SEQ ID NO: 22. The secretion unit in Pic comprises amino acid sequence from 1087 to 1372 of the sequence shown in SEQ ID NO:22; an example of the secretion unit in Pic is provided in SEQ ID NO:24.

The inventors prepared expression constructs containing nucleic acid encoding different lengths of Pic secretion unit peptide, using the same approach as outlined above for the Pet secretion unit experiments. These deletion variants were numbered in the same way as the Pet deletion constructs discussed above. Hence PicΔ*6 has amino acids 1035 to 1372 of Pic; PicΔ*12 has amino acids 1048 to 1372 of Pic; PicΔ*17 has amino acids 1065 to 1372; PicΔ*19 has amino acids 1079 to 1372 (294 amino acids); PicΔ*20 has amino acids 1087 to 1372 (286 amino acids).

They then introduced a gene encoding the ESAT6 protein to the expression construct, and investigated the expression and translocation of the ESAT6-Pic secretion unit peptide fusion in an appropriate host cell (E. coli TOP10 strain). The presence of ESAT6 polypeptide in concentrated cell culture supernatant was determined by Western blotting with anti-ESAT6 antibodies; consistently with the secretion result cleaved Pic β-barrel was detected in the OM fractions.

Therefore, the inventors have confirmed that a peptide comprising 286 amino acids (amino acids 1087 to 1372) or 294 amino acids (amino acids 1079 to 1372) of the Pic SPATE-class bacterial autotransporter polypeptide does function as a “secretion unit” peptide, and that a bacterial expression construct comprising nucleic acid sequence encoding that Pic secretion unit peptide can be used for the efficient expression and secretion of a protein of interest to the extracellular milieu.

### The Transmembrane β-Barrel Strands are Essential for Secretion

The inventors have demonstrated above that the minimal construct required for secreting proteins to the culture medium is the region encompassing the β-barrel, the linker that connects the barrel to the pore spanning α-helix and the α-helix. The inventors have termed this the ‘secretion unit’ peptide.

To determine precisely the elements required for the Secretion Unit to effect secretion of a protein to the culture medium we undertook two approaches: a random transposon strategy and a targeted insertion strategy. The transposon strategy utilised the random insertion of a nucleotide sequence which encoded a 19-amino acid sequence; there were three possible reading frames for this nucleotide sequence giving three possible amino acid insertions. In almost every case insertion of a linker into a surface loop was tolerated. Furthermore, deletion of loop 3 (pBADPetβΔL3) was found not to abolish secretion. This indicates that the amino acid sequence of the surface loops can be altered without affecting secretion of the protein. In contrast, insertion of the transposons insertions into the β-strands or turns in general abrogated secretion to the culture medium suggesting the integrity of these structures must be maintained for secretion to occur.

To confirm these observations the inventors used a targeted strategy where a nine-amino acid HA epitope was inserted into each β-strand and each surface loop. In general, insertions into the loops were tolerated and secretion to the culture medium was maintained; insertion into the β-strands was not tolerated and secretion was abrogated. Unexpectedly, some minor alterations in the β-strands can be tolerated—several insertions into β-strand 1 and 5 were tolerated; in each case the insertion compensates for loss of the native amino acid sequence by providing the necessary hydrophobic amino acids to complete the β-strand. This indicates limited alterations in the β-strands can be tolerated if the alterations maintain the integrity of the amphipathic β-strand (Table 2).

In the studies provided herein the inventors examined the role of the linker region. In some cases insertions into the linker region did not affect secretion of the protein to the culture medium. To test this further they made a construct in which the linker was increased in size (pBADPetβM7): secretion to the culture medium was maintained. It was found that the amino acid sequence of the linker region connecting the β-barrel to the α-helix can be altered and secretion is maintained. However, a linker sequence must be maintained as deletion of the linker abolishes secretion.

The inventors also looked to see if insertions into the α-helix affected secretion. Transposon insertions into the α-helix (pPetβEZ1) abolished secretion demonstrating that the integrity of the α-helix is required for secretion (Table 2).

### Conclusion

From the above data it can be seen that the inventors have identified a minimal region of Pet and Pic AT polypeptides which can function as a ‘secretion unit’. They have also determined that particular regions within the ‘secretion unit’ can also be altered. The findings presented herein demonstrate the minimal portion of Pet and Pic that can be used for secreting non-native proteins to the culture supernatant. Thus the data demonstrates the utility of a bacterial expression construct containing the secretion unit for a commercial protein expression system.

### Materials and Methods

### Description of the Pet-Based Secretion Cassette

The pet gene was synthesised de novo by GenScript and cloned into pBADHisA vector giving pBADPet construct. The pet gene sequence was codon optimised using E. coli codon usage gene and cleared of multiple restriction sites while unique restriction sites were engineered in this sequence to facilitate genetic manipulations. These procedures did not change native Pet protein translation. Thus the pet cassette has been made suitable for in-frame insertions of genes encoding recombinant proteins of interest and easy engineering of such features as affinity purification tags, protease cleavage sites and for convenient site mutagenesis. In this work, the recombinant DNA has been inserted between BglII-site on the right and one of the sites distributed across the passenger domain on the left as shown in FIG. 2. These insertions preserve N-terminal Pet signal sequence required for inner membrane translocation and C-terminal Pet translocation domain promoting outer membrane translocation. In principle, cloning between BglII-PstI sites could be the only construction step required to engineer secreted Pet fusion with a protein of interest but shorter secretion-proficient Pet C-terminus (Pet 1010-1295) can be engineered by simple PCR. Apart from using pBAD vector, the pet cassette could be further transferred in the preferred expression vector depending of the desired yield, genetic host background, vector copy number and induction regime. The inventors have used two other expression vectors, pASK-IBA33plus and pET22b, to produce Pet and fusion proteins. In the pASK-Pet, Pet is expressed from tetracycline promoter/operator and is induced by addition of a tetracycline derivative, anhydrotetracycline. The tetP/O expression system offers fine-tuned expression levels in dose-dependent manner. Expression from pASK vector is independent of host background but standard expression hosts are used for best result (such as E. coli TOP10 and BL21*). In pET22b vector the gene is placed under the transcriptional control of T7lac phage promoter (IPTG-inducible); pET vectors are used with a host carrying insertion of T7 phage polymerase (for example E. coli BL21(DE3) and derivatives) that is expressed from the IPTG-inducible lacUV5 promoter on the bacterial chromosome. In pBAD system, Pet is expressed from arabinose inducible P_{BAD} which can be additionally supressed by addition of glucose; the vector is used with ara-deficient strains such as E. coli TOP10. All these expression systems are commonly used in research and industry. In this work the inventors tested secretion of recombinant protein-Pet fusions using pET22b/BL21*(DE3) and pASK/TOP10 expression systems, both giving high levels of expression and secretion. The inventors used pBAD/arabinose expression system for the studies on mutagenesis of the secretion unit.

### Reagents, Media and Bacterial Strains.

A polyclonal rabbit antiserum generated towards the Pet passenger domain has been previously described (Eslava, C, F. Navarro-Garcia, J. R. Czeczulin, I. R. Henderson, A. Cravioto, and J. P. Nataro. 1998. Pet, an autotransporter enterotoxin from enteroaggregative Escherichia coli. Infection and Immunity 66:3155-3163). The Pet β-domain was cloned into the MBP-fusion tag expression vector pMAL-c2X (New England Biolabs, Herts, UK) and polyclonal rabbit antiserum was raised towards the MBP-Petβ fusion protein. Secondary goat anti-rabbit antibodies conjugated with alkaline phosphatase (AP) and AP-substrate (5-Bromo-4-chloro-3-indolylphosphate) were obtained from Sigma-Aldrich (UK). Polyclonal anti-ESAT6 and anti-RFP antibodies were purchased from Abeam. Restriction enzymes, DNA-modifying enzymes and T4 ligase were purchased from NEB, Invitrogen and Fermentas and were used according to the manufacturer's instructions. PCR was done using Phusion DNA Polymerase (Finnzymes).

E. coli strains TOP10 (F-mcrA Δ(mrr-hsdRMS-mcrBC) φ80lacZΔM15 ΔlacX74 nupG recA1 araD139 Δ(ara-leu)7697 galE15 galK16 rpsL(Str^{R}) endA1 λ⁻; Invitrogen), NEB 5αF′I^{q} (F′proA⁺B⁺lacI^{q} Δ(lacZ)M15zzf::Tn10 (Tet^{R})/fhuA2Δ(argF-lacZ)U169 phoA glnV44 φ80Δ(lacZ)M15 gyrA96 recA1 endA1 thi-1 hsdR17; NEB), RLG221 (recA56 araD139 (ara-leu)7697 lacX74 galU galK hsdR strA), JM110 (rpsL thr leu thi lacY galK galT ara tonA tsx dam dcm glnV44 Δ(lac-proAB) e14-[F′ traD36 proAB⁺lacI^{q} lacZΔM15] hsdR17(r_{K}⁻m_{K}⁺); NEB) and HB101 (Promega) were used for cloning. E. coli TOP10, TOP10 dsbA (Km^{r}), BL21*(F⁻ ompThsdS_{B} (r_{B}⁻m_{B}⁻) gal dcm rne131 (DE3) (Novagen) and Salmonella enterica Typhimurium SL1344 strains were used for protein expression. Bacterial strains were grown at 37° C. in Luria-Bertani liquid and solid (3% agar) media supplemented with carbenicillin (100 and 80 μg/ml respectively) for plasmid maintenance. For expression from pBAD vector, bacteria were grown at 37° C. in Luria-Bertani (LB) broth and where necessary, the growth medium was supplemented with 100 μg mL⁻¹ ampicillin, 2% D-glucose, or 0.02% L-arabinose.

### DNA and Electrophoresis Techniques.

Standard techniques were employed for all recombinant DNA manipulations and electrophoresis procedures, including sodium dodecyl sulphate-polyacrylamide electrophoresis (SDS-PAGE) (Sambrook, supra). Plasmid DNA was isolated using the Qiagen Spin miniprep kit (Qiagen, UK), and PCRs and digests were purified using the Qiaquick PCR purification kit or Gel extraction kit (Qiagen) according to the manufacturer's instructions. Alternatively, small DNA fragments arising post-digestion or from PCR were separated on a 7.5% polyacrylamide gel, excised and eluted overnight at 4° C. with Elution Buffer (10 mM Tris-HCl pH 7.5, 50 mM NaCl, 1 mM EDTA pH 8.0). The DNA was then ethanol-precipitated and resuspended in milliQ H₂O.

### Plasmid Construction.

Plasmids used and constructed for mutagenesis in Pet secretion unit. Plasmids used in this part of study are listed in Table 3. A codon optimized pet gene was synthesized de novo by GenScript and cloned into pBADHisA (Invitrogen) to create pBADPet (Leyton, D. L., M. d. G. De Luna, Y. R. Sevastsyanovich, K. Tveen Jensen, D. F. Browning, A. Scott-Tucker, and I. R. Henderson. (2010) FEMS Microbiology Letters 311:133-139). Distinct fragments flanked by specific restriction sites and comprising sequence coding for the Pet translocator domain with defined HA epitope tag insertions, various deletions and sequence mutations were synthesized de novo and cloned into pUC57 (GenScript). pUC57 comprising these fragments were digested with the appropriate restriction enzymes and subcloned into pBADPet, pre-digested with the same restriction enzymes, to create pBADPet derivatives containing these distinct fragments (Table 3).

Plasmids used and constructed for secretion work and for defining Pet and Pic secretion units. pBADPet (Leyton supra) was used as a source of pet gene (codon optimised). For expression experiments, pet was cloned into pASK-IBA33plus (IBA BioTAGnology) under the control of tetracycline promoter/operator and into pET22b (Novagen) under the control of T7lac promoter. To generate pASK-Pet, the pet gene was amplified from pBADPet by PCR using BsaI-pet-F and HindIII-pet-R primers (Table 4) and cloned between BsaI and HindIII sites in pASK-IBA33plus. To construct pET-Pet, pet gene was excised from pBADPet as an NdeI-HindIII fragment and cloned into pET22b pre-digested with the same enzymes. In pASK-His₆-Pet and pASK-His₆-Pet-ΔD1, the nucleotide sequence encoding His₆-tag has been incorporated in pet gene after the signal sequence. To construct these plasmids, an approximately ˜100 bp pet fragment gene was amplified from pASK-Pet using primers SacI-pet-F/PetSS-BglII-AflII-BstBI-R and the resulting amplicon was cloned in pASK-Pet between SacI/BglII or SacI/BstBI sites. pASK-Pet* was constructed by replacing PstI-HindIII fragment of pet gene in pASK-Pet with equivalent fragment from pBADPet*, which contains mutations resulting in N1018G and D1115G substitutions in Pet translocation domain. These mutations result in production of non cleaved Pet protein.

To construct pet chimeras with heterologous genes, the latter were amplified by PCR using relevant DNA templates and appropriate primer pairs listed in Tables 3 and 4. For sapA, pmp17, latA, bmaa1263 and yapA only part of the gene corresponding to the predicted extracellular protein domain was used to insert into pet (Table 3). The PCR-amplified heterologous genes were cloned between BglII/ClaI, BglII/BstBI and BglII/PstI sites in pet gene in pASK-Pet and subsequently the full length chimeric fusions were cloned into the NdeI-HindIII sites of pET22b if expression in the BL217T7 system was to be tested. Primers used for these clonings are listed in Table 4. To create the equivalent constructs encoding His₆-tagged fusion proteins, the chimeric sequences constructed in pASK-Pet were sub-cloned into pASK-His₆-Pet as BglII-HindIII fragments. pASK-ESAT6-Pet* and pASK-mCherry-Pet* were constructed in the same way as pASK-ESAT6-Pet-BB and pASK-mCherry-Pet-BP (above) but using pASK-Pet* as a vector for inserting relevant PCR-amplified genes. pASK-Ag85B-ESAT6-Pet was constructed by inserting PCR-amplified esxA gene (ESAT6) between BstBI-PstI sites in pASK-Ag85B-Pet-BB. Constructs pASK-ESAT6-Pet Δ*1 to Δ*20 were made by replacing the PstI-HindIII fragment in pASK-ESAT6-Pet-BP with the shorter pet gene fragments generated by PCR with one of the forward primers (PstI-TSYQ-del1-F to PstI-YKAF-del20-F) and HindIII-pet-R as a reverse primer (Table 4). The equivalent constructs encoding ESAT6-Pic chimeras were generated by replacing the PstI-HindIII pet fragment in pASK-ESAT6-Pet-BP with the pic fragment amplified from pPid using one of the forward primers (SbfI-FKAG-Pic-del6-F to SbfI-YKNF-Pic-del20-F) and HindIII-Pic-end-R as a reverse primer. To mutate codons determining 1974, W985, L987 and G989 in pASK-ESAT6-PetΔ*6, the site directed mutagenesis primers (Table 4) were used in two-step (overlap) PCR with the BglII-ESAT6-F and HindIII-pet-R primers. All constructs generated in this study were sequenced to confirm the veracity of the nucleotide modifications.

### Secretion Profiles and Outer Membrane Preparations.

Cultures of E. coli TOP10, TOP10 dsbA, BL21*(DE3) and SL1344 containing recombinant test constructs and appropriate vector controls were grown overnight in 5 ml LB supplemented with carbenicillin at 37° C. with aeration (180 rpm). The overnight cultures were diluted at 1/100 in 50 ml of fresh LB medium (with 100 μg/ml carbenicillin) in 250 ml conical flasks and grown at 37° C. with aeration (180 rpm) until OD_{600 nm} of approximately 0.5. Protein expression was induced by adding anhydrotetracycline (aTc, 200 μg/L final concentration) or IPTG (0.5 mM) depending on the expression system used and the cultures were grown for further 2 h. The culture OD_{600 nm} values were equalised by diluting with L-broth and 20 ml of these culture samples were harvested by centrifugation. The spent media (supernatant) was filtered through 0.2 μm and secreted proteins were precipitated by adding 1/10 volume of ice-cold 100% (w/v) TCA. After 45 min incubation on ice, precipitated proteins were pelleted by centrifugation for 45 min at 14,000 rpm at 4° C. The pellets were washed once with ice-cold methanol and pelleted as above. The pellets were dried for 30 min using Speed Vac and resuspended in 2×SDS-PAGE loading dye with 10% saturated Tris buffer. Five to 10 μl of the secreted protein samples were analysed on SDS-PAGE (10-15% polyacrylamide). Bacterial pellets from the same experiment were used to prepare cell envelope fractions as previously described (Henderson et al (1997) FEMS Microbiol. Letters 149, 115-120). Briefly, cells were resuspended in 10 ml Tris buffer, pH 7.4, and broken by sonication. Unbroken cells and debris were removed by centrifugation (10,000 rpm, 15 min, 4° C.) while supernatants were centrifuged for 1.5 h at 18,000 rpm at 4° C. to pellet cell envelopes. The outer membrane fraction was then extracted with 2% (v/v) Triton X-100 and harvested by centrifugation as before. The outer membrane fraction was washed extensively in 10 mM Tris-HCl (pH 7.4) to remove the detergent. The outer membrane proteins were resuspended in SDS-PAGE loading dye and analysed on 12% SDS-PAGE.

Pet Biogenesis Using pBAD/Arabinose Induction System.

Growth and expression of Pet from E. coli TOP10 transformed with various pBADPet derivatives (Table 3) was performed as previously described (Leyton 2010 supra). Briefly, overnight E. coli LB cultures, supplemented with glucose, were diluted 1:100 into fresh medium and grown to an OD₆₀₀=0.5. The bacteria were pelleted by centrifugation (10,000 g, 4° C., 10 min), washed with LB broth, pelleted as before, resuspended in LB broth supplemented with arabinose and grown for an additional 1 h. The OD₆₀₀ of cultures were normalized to allow comparison of secreted protein levels, pelleted as before, and the supernatants were then filtered through 0.22 μm⁻¹ pore-size filters (Millipore, USA). TCA precipitation of culture supernatants and extraction of outer membrane proteins were done as above.

### Western Blotting.

Secretion of Pet and chimeras was assessed by Western blotting using polyclonal rabbit anti-Pet (1/5000), anti-ESAT-6 (1/2000) or anti-RFP (1/2000) as primary antibodies and goat anti-rabbit alkali phosphatase-conjugated antibodies (1/10000) as secondary antibodies. Blocking of blots and primary antibody incubation was done in 1×PBS, 0.05% Tween 20, 5% dry skimmed milk. Blots were washed in 1×PBS, 0.05% Tween 20 buffer. Primary antibody incubation was usually performed overnight at 4° C. while secondary antibody incubation was for 1-2 h at room temperature. The blots were developed using NBT/BCIP substrate solution (Sigma).

### EZ-Tn5 In-Frame Linker Insertion.

The EZ-Tn5 in-frame linker insertion kit (Epicentre Biotechnologies, USA) was used according to the manufacturer's instructions to introduce a 19 amino acid linker randomly into the pet open reading frame. Briefly, an in vitro transposon reaction was prepared by mixing the target DNA (pCEFNI; Table 3) with EZ-Tn5 transposase and EZ-Tn5 transposon, which contains a Kanamycin resistance cassette between two NotI restriction sites. Transposon reactions were stopped and immediately transformed into E. coli TOP10. Kanamycin resistant transformants harbouring insertions within the pet translocator domain were identified through colony PCR using primers 3-barrelFor (5′-AAAATGCATGTAAGGATGTCTTCAAAACTGAAACACAGA-3′) and β-barrelRev (5′-TCACTCATTAGGCACCCCAG-3), and size analysis of PCR products. Plasmid DNA was isolated from these transformants, digested with NotI to excise the Kanamycin cassette, purified and the backbone re-ligated to generate clones with a single NotI restriction site and a 57 nucleotide (19 amino acids) insertion into all three reading frames. Ligations were transformed into E. coli HB101 and selected using Ampicillin, the antibiotic marker present in pCEFNL Plasmid DNA was extracted from Kanamycin sensitive and Ampicillin resistant transformants and sequenced using primers β-barrelFor and β-barrelRev to map the linker insertion sites within the Pet translocator domain.

### Flow Cytometry.

Propidium iodide (PI) and Bis-(1,3-dibutylbarbituric acid) trimethine oxonol (Bis-oxonol or BOX) were purchased from Sigma. For analysis of viability, bacterial cells (˜10⁵-10⁶) were diluted in 1 ml filter-sterilised Dulbecco's PBS supplemented with 10 μl of working solutions of PI and BOX (5 and 10 μg/ml respectively) and analysed immediately on FACSAria II (BD Biosciences) using 488 nm laser. Side and forward scatter data and fluorescence data from 104 particles were collected. Optical filters used to measure green and red fluorescence were 502LP, 530/30BP (FITC) and 610LP, 616/23BP (PE-Texas Red) respectively. Discriminator on forward scatter was adjusted to exclude small particle noise. To analyse surface localisation of proteins by indirect flow cytometry, cells were washed in PBS and incubated at room temperature (RT) with PBS+ 1% BSA to block non-specific binding. Cells were then incubated, for 1 h at RT, with relevant primary antibody diluted in the same buffer (anti-Pet, 1:500; anti-ESAT6, 1:500; anti-mCherry, 1:800) followed by 3 washes in PBS and final incubation with Alexa Fluor® 488 goat anti-rabbit IgG (1:500; Invitrogen) under the same conditions. Cells were washed as before and analysed on FACSAria II as described above.

### Immunofluorescence.

Immunofluorescent detection of proteins in live or fixed bacterial cells was done as previously described (Leyton et al (2011) J Biol Chem 286:42283-91). Briefly, poly-L-lysine-coated coverslips loaded with either fixed or live cells were washed three times with PBS, and nonspecific binding sites were blocked for 1 h in PBS containing 1% BSA (Europa Bioproducts). Coverslips were incubated with the appropriate antibody for 1 h, washed three times with PBS, and incubated for an additional 1 h with Alexa Fluor® 488 goat anti-rabbit IgG. The coverslips were then washed three times with PBS, mounted onto glass slides, and visualized using either phase contrast or fluorescence using a Zeiss Axiolmager Z2 microscope (100× objective) and an AxioCam MRm camera. Exposure time was 40 ms.

### CD Analysis of Protein Folding.

Far-UV CD measurements were collected from 190 to 260 nm on a JASCO J-715 spectropolarimeter at room temperature, as described previously (Leyton 2011 supra). Briefly, For CD analysis purified proteins were buffer exchanged into 10 mM Sodium Phosphate, pH 8.0; readings were taken with a 1-mm path length cell, 2-nm bandwidth, 1-nm increments, 2-s response, 100 nm/min scanning speed, and in continuous scanning mode. 12 accumulations for folded proteins were averaged, and the spectrum was subtracted for buffer contribution. Protein structures were modelled in Swiss-Model or Phyre and were visualised using PyMol. Secondary structure predictions were done with PsiPred.

### Alkaline Phosphatase Assay.

The Garen and Levinthal (1960) Biochim Biophys Acta 38, 470-483) assay of AP activity was used based on conversion of p-nitrophenylphosphate (pNPP) substrate into yellow product with absorbance at 410 nm. Briefly, 1 ml of 3 mM pNPP solution was mixed with 2 ml of 1.5 M Tris-HCl, pH 8.0, and incubated in 25° C. water bath for 5 min before adding 0.1 ml of concentrated culture supernatants or cell lysates. After 1 h incubation in 25° C. water bath, OD 410 nm was determined.

TABLE 1
Mass spectrometry analysis of some recombinant protein fusions with Pet.

| Fusion | | # | # | # | | |
|---|---|---|---|---|---|---|
| protein | Coverage | PSMs | Peptides | AAs | Score | Description |
| YapA- | 35.24 | 1357 | 55 | 1430 | 5376.35 | putative autotransporter protein |
| Pet-BP | | | | | | [Yersinia pestis CA88-4125] |
| | 13.82 | 638 | 24 | 1295 | 1852.07 | RecName: Full = Serine protease pet |
| | | | | | | autotransporter; Contains: |
| | | | | | | RecName: Full = Serine protease |
| | | | | | | pet; AltName: Full = Plasmid- |
| | | | | | | encoded toxin pet; Contains: |
| | | | | | | RecName: Full = Serine protease pet |
| | | | | | | translocator; Flags: Precursor |
| Ag85B- | 41.62 | 1522 | 63 | 1295 | 5215.18 | RecName: Full = Serine protease pet |
| Pet-BB | | | | | | autotransporter; Contains: |
| | | | | | | RecName: Full = Serine protease |
| | | | | | | pet; AltName: Full = Plasmid- |
| | | | | | | encoded toxin pet; Contains: |
| | | | | | | RecName: Full = Serine protease pet |
| | | | | | | translocator; Flags: Precursor |
| | 8.42 | 72 | 2 | 285 | 255.16 | Chain A, Mycobacterium |
| | | | | | | Tuberculosis Antigen 85b With |
| | | | | | | Trehalose |
| | 7.38 | 72 | 2 | 325 | 255.16 | secreted antigen Ag85B |
| | | | | | | [Mycobacterium tuberculosis] |
| Pertactin- | 8.80 | 572 | 18 | 1295 | 1422.29 | RecName: Full = Serine protease pet |
| Pet | | | | | | autotransporter; Contains: |
| | | | | | | RecName: Full = Serine protease |
| | | | | | | pet; AltName: Full = Plasmid- |
| | | | | | | encoded toxin pet; Contains: |
| | | | | | | RecName: Full = Serine protease pet |
| | | | | | | translocator; Flags: Precursor |
| | 22.63 | 325 | 12 | 539 | 1237.01 | Chain A, The Structure Of |
| | | | | | | Bordetella Pertussis Virulence |
| | | | | | | Factor P.69 Pertactin |
| Pmp17- | 39.15 | 1255 | 58 | 1295 | 4062.94 | RecName: Full = Serine protease pet |
| Pet-BB | | | | | | autotransporter; Contains: |
| | | | | | | RecName: Full = Serine protease |
| | | | | | | pet; AltName: Full = Plasmid- |
| | | | | | | encoded toxin pet; Contains: |
| | | | | | | RecName: Full = Serine protease pet |
| | | | | | | translocator; Flags: Precursor |
| | 13.71 | 237 | 13 | 839 | 664.93 | polymorphic outer membrane |
| | | | | | | protein [Chlamydophila abortus |
| | | | | | | S26/3] |
| ESAT6- | 40.93 | 1471 | 65 | 1295 | 4929.59 | RecName: Full = Serine protease pet |
| Pet-BB | | | | | | autotransporter; Contains: |
| | | | | | | RecName: Full = Serine protease |
| | | | | | | pet; AltName: Full = Plasmid- |
| | | | | | | encoded toxin pet; Contains: |
| | | | | | | RecName: Full = Serine protease pet |
| | | | | | | translocator; Flags: Precursor |
| | 38.30 | 97 | 2 | 94 | 530.53 | Chain B, Structure Of The Cfp10- |
| | | | | | | Esat6 Complex From |
| | | | | | | Mycobacterium Tuberculosis |
| | 37.89 | 59 | 2 | 95 | 310.30 | 6 kDa early secreted antigenic |
| | | | | | | protein [Mycobacterium ulcerans] |
| ESAT6- | 33.36 | 1175 | 46 | 1295 | 3500.18 | RecName: Full = Serine protease pet |
| Pet-BP | | | | | | autotransporter; Contains: |
| | | | | | | RecName: Full = Serine protease |
| | | | | | | pet; AltName: Full = Plasmid- |
| | | | | | | encoded toxin pet; Contains: |
| | | | | | | RecName: Full = Serine protease pet |
| | | | | | | translocator; Flags: Precursor |
| | 38.30 | 130 | 2 | 94 | 604.17 | Chain B, Structure Of The Cfp10- |
| | | | | | | Esat6 Complex From |
| | | | | | | Mycobacterium Tuberculosis |
| | 37.89 | 85 | 2 | 95 | 370.05 | 6 kDa early secreted antigenic |
| | | | | | | protein [Mycobacterium ulcerans] |
| | 37.89 | 67 | 2 | 95 | 363.46 | 6 kDa early secretory antigenic |
| | | | | | | target [Mycobacterium kansasii] |
| | 37.89 | 67 | 2 | 95 | 362.59 | Esat6 [Mycobacterium riyadhense] |
| SapA- | 12.66 | 198 | 24 | 1295 | 542.16 | RecName: Full = Serine protease pet |
| Pet-BP | | | | | | autotransporter; Contains: |
| | | | | | | RecName: Full = Serine protease |
| | | | | | | pet; AltName: Full = Plasmid- |
| | | | | | | encoded toxin pet; Contains: |
| | | | | | | RecName: Full = Serine protease pet |
| | | | | | | translocator; Flags: Precursor |
| | 4.62 | 51 | 3 | 931 | 138.88 | Flagellar protein [Salmonella |
| | | | | | | enterica subsp. enterica serovar |
| | | | | | | Saintpaul str. SARA23] |
| mCherry- | 10.19 | 123 | 17 | 1295 | 325.03 | RecName: Full = Serine protease pet |
| Pet-BP | | | | | | autotransporter; Contains: |
| | | | | | | RecName: Full = Serine protease |
| | | | | | | pet; AltName: Full = Plasmid- |
| | | | | | | encoded toxin pet; Contains: |
| | | | | | | RecName: Full = Serine protease pet |
| | | | | | | translocator; Flags: Precursor |
| | 47.88 | 45 | 16 | 236 | 161.22 | gb\|AAV52164.1\| monomeric red |
| | | | | | | fluorescent protein [synthetic |
| | | | | | | construct] |

TABLE 2
Pet derivatives permissive and non-permissive for secretion.

| | Location of | |
|---|---|---|
| Plasmid | mutation | Secretion |
| Control vectors | | |
| pSPORT1 | NA | No |
| pCEFN1 | NA | Yes |
| pBADHisA | NA | No |
| pBADPet | NA | Yes |
| EZTn5 insertion | | |
| mutants | | |
| pPetβEZ1 | α-helix | No |
| pPetβEZ2 | Linker | No |
| pPetβEZ3A/B | Linker | Yes x2 |
| pPetβEZ4 | β1 | Yes |
| pPetβEZ5 | β1 | No |
| pPetβEZ6 | β2 | No |
| pPetβEZ7 | β2 | No |
| pPetβEZ8 | β2 | No |
| pPetβEZ9 | T1 | No |
| pPetβEZ10A/B/C | β3 | No x3 |
| pPetβEZ11A/B/C/D/E | β3 | No x5 |
| pPetβEZ12 | β3-L2 | Yes |
| pPetβEZ13 | L2 | Yes |
| pPetβEZ14 | β4 | No |
| pPetβEZ15A/B/C | β4 | No x3 |
| pPetβEZ16 | T2 | No |
| pPetβEZ17 | β5 | No |
| pPetβEZ18 | β5 | Yes |
| pPetβEZ19 | β5-L3 | No |
| pPetβEZ20A/B/C/D/E | β7 | No x5 |
| pPetβEZ21 | L4 | Yes |
| pPetβEZ22 | L4-β8 | Yes |
| pPetβEZ23A/B | β8 | No x2 |
| pPetβEZ24 | L5 | No |
| pPetβEZ25 | β10 | No |
| pPetβEZ26 | β10 | No |
| pPetβEZ27 | β11 | No |
| HA-epitope mutants | | |
| pBADPetβHA1 | β1 | Yes |
| pBADPetβHA2 | β1 | No |
| pBADPetβHA3 | β2 | No |
| pBADPetβHA4 | β3 | No |
| pBADPetβHA5 | β4 | No |
| pBADPetβHA6 | β5 | No |
| pBADPetβHA7 | β6 | No |
| pBADPetβHA8 | β7 | No |
| pBADPetβHA9 | β8 | No |
| pBADPetβHA10 | β9 | No |
| pBADPetβHA11 | β10 | No |
| pBADPetβHA12 | β11 | No |
| pBADPetβHA13 | β12 | No |
| pBADPetβHA14 | β1-L1 | No |
| pBADPetβHA15 | L2 | Yes |
| pBADPetβHA16 | L3 | Yes |
| pBADPetβHA17 | L4 | Yes |
| pBADPetβHA18 | L5 | Yes |
| pBADPetβHA19 | L6 | Yes |
| Deletion mutants | | |
| pBADPetβΔL3 | L3 | Yes |
| pBADPetβΔL4 | L4 | No |
| pBADPetβΔL5 | L5 | No |
| pBADPetβΔLinker | Linker | No |
| pBADPetβΔHelix | Helix | No |
| Miscellaneous | | |
| mutants | | |
| pBADPetβM1 | Linker | No |
| pBADPetβM2 | β-barrel surface | No |
| pBADPetβM3 | β-barrel interior | No |
| pBADPetβM4 | β-barrel interior | No |
| pBADPetβM5 | β-barrel interior | No |
| pBADPetβM6 | β-barrel cleavage | No |
| | site | |
| pBADPetβM7 | Linker | Yes |
| pBADPetβM8 | Linker | No |
| pBADPetβM9 | Helix | No |

TABLE 3
Plasmids used in this study.

| Plasmid | | | |
|---|---|---|---|
| Cloning/expression | | | |
| vectors | Relevant description | | Reference |
| pSPORT1 | Cloning vector | | Invitrogen |
| pCEFN1 | pSPORT1 derivative expressing Pet from its original | | Eslava et al. |
| | promoter | | (1998) |
| | | | Infection and |
| | | | Immunity 66, |
| | | | 3155-3163 |
| pBADHisA | Arabinose-inducible expression vector | | Invitrogen |
| pBADPet | pBADHisA derivative expressing de novo | | GenScript/ |
| | synthesized Pet | | This study |
| pUC57 | Cloning vector | | GenScript |
| pASK-IBA33plus | Expression vector, tet promoter/operator, Amp^{r} | | IBA |
| | | | BioTAGnology |
| pET22b | Expression vector, T7lac promoter, Amp^{r} | | Novagen |
| EZTn5 insertion | | Location of | |
| mutants | | linker | |
| pPetβEZ1 | EZTn5 linker between L₁₀₂₀-N₁₀₂₁ in Pet | α-helix | This study |
| | from pCEFN1 | | |
| pPetβEZ2 | EZTn5 linker between L₁₀₂₇-R₁₀₂₈ in Pet | Linker | This study |
| | from pCEFN1 | | |
| pPetβEZ3A/B | EZTn5 linker between G₁₀₃₂-E₁₀₃₃ in Pet | Linker | This study |
| | from pCEFN1 (coding frame + 1 for both) | | |
| pPetβEZ4 | EZTn5 linker between A₁₀₃₈-R₁₀₃₉ in Pet | β1 | This study |
| | from pCEFN1 (coding frame 0) | | |
| pPetβEZ5 | EZTn5 linker between A₁₀₃₈-R₁₀₃₉ in Pet | β1 | This study |
| | from pCEFN1 (coding frame + 2) | | |
| pPetβEZ6 | EZTn5 linker between D₁₀₅₃-N₁₀₅₄ in Pet | β2 | This study |
| | from pCEFN1 | | |
| pPetβEZ7 | EZTn5 linker between T₁₀₅₆-H₁₀₅₇ in Pet | β2 | This study |
| | from pCEFN1 | | |
| pPetβEZ8 | EZTn5 linker between V₁₀₆₀-G₁₀₆₁ in Pet | β2 | This study |
| | from pCEFN1 | | |
| pPetβEZ9 | EZTn5 linker between L₁₀₆₈-D₁₀₆₉ in Pet | T1 | This study |
| | from pCEFN1 | | |
| pPetβEZ10A/B/C | EZTn5 linker between L₁₀₇₃-F₁₀₇₄ in Pet | β3 | This study |
| | from pCEFN1 (coding frame 0 for all | | |
| | three) | | |
| pPetβEZ11A/B/C/D/E | EZTn5 linker between T₁₀₈₀-Y₁₀₈₁ in Pet | β3 | This study |
| | from pCEFN1 (coding frame + 2 for all | | |
| | five) | | |
| pPetβEZ12 | EZTn5 linker between G₁₀₈₇-S₁₀₈₈ in Pet | β3-L2 | This study |
| | from pCEFN1 | | |
| pPetβEZ13 | EZTn5 linker between A₁₀₉₀-F₁₀₉₁ in Pet | L2 | This study |
| | from pCEFN1 | | |
| pPetβEZ14 | EZTn5 linker between A₁₁₀₀-G₁₁₀₁ in Pet | β4 | This study |
| | from pCEFN1 | | |
| pPetβEZ15A/B/C | EZTn5 linker between A₁₁₀₄-S₁₁₀₅ in Pet | β4 | This study |
| | from pCEFN1 (coding frame + 2 for all | | |
| | three) | | |
| pPetβEZ16 | EZTn5 linker between S₁₁₁₀-G₁₁₁₁ in Pet | T2 | This study |
| | from pCEFN1 | | |
| pPetβEZ17 | EZTn5 linker between K₁₁₁₉-Y₁₁₂₀ in Pet | β5 | This study |
| | from pCEFN1 | | |
| pPetβEZ18 | EZTn5 linker between D₁₁₂₄-N₁₁₂₅ in Pet | β5 | This study |
| | from pCEFN1 | | |
| pPetβEZ19 | EZTn5 linker between T₁₁₂₈-A₁₁₂₉ in Pet | β5-L3 | This study |
| | from pCEFN1 | | |
| pPetβEZ20A/B/C/D/E | EZTn5 linker between P₁₁₆₄-Q₁₁₆₅ in Pet | β7 | This study |
| | from pCEFN1 (coding frame + 1 for all | | |
| | five) | | |
| pPetβEZ21 | EZTn5 linker between L₁₁₈₇-T₁₁₈₈ in Pet | L4 | This study |
| | from pCEFN1 | | |
| pPetβEZ22 | EZTn5 linker between M₁₁₈₉-K₁₁₉₀ in Pet | L4-β8 | This study |
| | from pCEFN1 | | |
| pPetβEZ23A/B | EZTn5 linker between S₁₂₀₈-F₁₂₀₉ in Pet | β8 | This study |
| | from pCEFN1 (coding frame + 2 for both) | | |
| pPetβEZ24 | EZTn5 linker between E₁₂₃₃-T₁₂₃₄ in Pet | L5 | This study |
| | from pCEFN1 | | |
| pPetβEZ25 | EZTn5 linker between L₁₂₅₄-M₁₂₅₅ in Pet | β10 | This study |
| | from pCEFN1 (coding frame 0)^{b} | | |
| pPetβEZ26 | EZTn5 linker between L₁₂₅₄-M₁₂₅₅ in Pet | β10 | This study |
| | from pCEFN1 (coding frame + 2)^{b} | | |
| pPetβEZ27 | EZTn5 linker between L₁₂₇₁-E₁₂₇₂ in Pet | β11 | This study |
| | from pCEFN1 | | |
| HA-epitope mutants | | | |
| pBADPetβHA1 | HA-epitope between G₁₀₃₅-A₁₀₃₆ in Pet, | β1 | This study |
| | 387-bp HpaI/KpnI fragment subcloned | | |
| | into pBADPet | | |
| pBADPetβHA2 | HA-epitope between M₁₀₄₁-S₁₀₄₂ in Pet, | β1 | This study |
| | 385-bp SalI/EagI fragment subcloned | | |
| | into pBADPet | | |
| pBADPetβHA3 | HA-epitope between V₁₀₅₈-Q₁₀₅₉ in Pet, | β2 | This study |
| | 387-bp HpaI/KpnI fragment subcloned | | |
| | into pBADPet | | |
| pBADPetβHA4 | HA-epitope between V₁₀₇₇-T₁₀₇₈ in Pet, | β3 | This study |
| | 387-bp HpaI/KpnI fragment subcloned | | |
| | into pBADPet | | |
| pBADPetβHA5 | HA-epitope between G₁₁₀₁-L₁₁₀₂ in Pet, | β4 | This study |
| | 387-bp HpaI/KpnI fragment subcloned | | |
| | into pBADPet | | |
| pBADPetβHA6 | HA-epitope between V₁₁₂₁-H₁₁₂₂ in Pet, | β5 | This study |
| | 387-bp NgoMIV/AatII fragment | | |
| | subcloned into pBADPet | | |
| pBADPetβHA7 | HA-epitope between G₁₁₄₇-A₁₁₄₈ in Pet, | β6 | This study |
| | 387-bp NgoMIV/AatII fragment | | |
| | subcloned into pBADPet | | |
| pBADPetβHA8 | HA-epitope between Y₁₁₇₀-G₁₁₇₁ in Pet, | β7 | This study |
| | 387-bp NgoMIV/AatII fragment | | |
| | subcloned into pBADPet | | |
| pBADPetβHA9 | HA-epitope between R₁₂₀₀-T₁₂₀₁ in Pet, | β8 | This study |
| | 387-bp NgoMIV/AatII fragment | | |
| | subcloned into pBADPet | | |
| pBADPetβHA10 | HA-epitope between R₁₂₁₉-A₁₂₂₀ in Pet, | β9 | This study |
| | 444-bp KpnI/EcoRI fragment subcloned | | |
| | into pBADPet | | |
| pBADPetβHA11 | HA-epitope between R₁₂₅₂-M₁₂₅₃ in Pet, | β10 | This study |
| | 444-bp KpnI/EcoRI fragment subcloned | | |
| | into pBADPet | | |
| pBADPetβHA12 | HA-epitope between E₁₂₇₄-K₁₂₇₅ in Pet, | β11 | This study |
| | 315-bp AatII/EcoRI fragment subcloned | | |
| | into pBADPet | | |
| pBADPetβHA13 | HA-epitope between N₁₂₈₈-A₁₂₈₉ in Pet, | β12 | This study |
| | 213-bp EcoRI/HindIII fragment | | |
| | subcloned into pBADPet | | |
| pBADPetβHA14 | HA-epitope between S₁₀₄₆-A₁₀₄₇ in Pet, | β1-L1 | This study |
| | 387-bp HpaI/KpnI fragment subcloned | | |
| | into pBADPet | | |
| pBADPetβHA15 | HA-epitope between S₁₀₈₈-D₁₀₈₉ in Pet, | L2 | This study |
| | 387-bp HpaI/KpnI fragment subcloned | | |
| | into pBADPet | | |
| pBADPetβHA16 | HA-epitope between F₁₁₃₁-A₁₁₃₂ in Pet, | L3 | This study |
| | 387-bp NgoMIV/AatII fragment | | |
| | subcloned into pBADPet | | |
| pBADPetβHA17 | HA-epitope between G₁₁₈₄-M₁₁₈₅ in Pet, | L4 | This study |
| | 387-bp NgoMIV/AatII fragment | | |
| | subcloned into pBADPet | | |
| pBADPetβHA18 | HA-epitope between R₁₂₃₇-D₁₂₃₈ in Pet, | L5 | This study |
| | 444-bp KpnI/EcoRI fragment subcloned | | |
| | into pBADPet | | |
| pBADPetβHA19 | HA-epitope between G₁₂₇₉-K₁₂₈₀ in Pet, | L6 | This study |
| | 213-bp EcoRI/HindIII fragment | | |
| | subcloned into pBADPet | | |

| | | |
|---|---|---|
| Deletion mutants | | |
| pBADPetβΔL3 | Loop 3 deletion in Pet, 351-bp NgoMIV/AatII | This study |
| | fragment subcloned into pBADPet | |
| pBADPetβΔL4 | Loop 4 deletion in Pet, 339-bp NgoMIV/AatII | This study |
| | fragment subcloned into pBADPet | |
| pBADPetβΔL5 | Loop 5 deletion in Pet, 378-bp KpnI/EcoRI fragment | This study |
| | subcloned into pBADPet | |
| pBADPetβΔLinker | Linker deletion in Pet, 345-bp HpaI/KpnI fragment | This study |
| | subcloned into pBADPet | |
| pBADPetβΔHelix | Helix deletion in Pet, 331-bp SalI/EagI fragment | This study |
| | subcloned into pBADPet | |
| pBADPetβΔHSF | HSF deletion in Pet, 432-bp SalI/NgoMIV fragment | This study |
| | subcloned into pBADPet | |
| Miscellaneous | | |
| mutants | | |
| pBADPetβM1 | Linker residues changed to lysines in Pet, 358-bp | This study |
| | SalI/EagI fragment subcloned into pBADPet | |
| pBADPetβM2 | Surface hydrophobic residues changed to glycine in | This study |
| | Pet, 564-bp NgoMIV/EcoRI fragment subcloned into | |
| | pBADPet | |
| pBADPetβM3 | Hydrophobic tract residues mutated in Pet, 1035-bp | This study |
| | SalI/EcoRI fragment subcloned into pBADPet | |
| pBADPetβM4 | Pet barrel interior filled in to occlude pore, 849-bp | This study |
| | HpaI/EcoRI fragment subcloned into pBADPet | |
| pBADPetβM5 | Pet barrel interior made less occluded, 495-bp | This study |
| | KpnI/EcoRI fragment subcloned into pBADPet | |
| pBADPetβM6 | Mutation of putative cleavage site amino acids | This study |
| | Asn₁₀₁₈/Asp₁₁₁₅ in Pet, 624-bp SalI/KpnI fragment | |
| | subcloned into pBADPet | |
| pBADPetβM7 | Linker duplicated in Pet, 388-bp SalI/EagI fragment | This study |
| | subcloned into pBADPet | |
| pBADPetβM8 | Linker residues changed to glycines in Pet, 358-bp | This study |
| | SalI/EagI fragment subcloned into pBADPet | |
| pBADPetβM9 | Helix duplicated in Pet, 400-bp SalI/EagI fragment | This study |
| | subcloned into pBADPet | |
| Secretion studies | | |
| pPic1 | pACYC184 plasmids with insertion of pic gene from | Henderson et |
| | E. coli 042 | al., 1999 |
| pASK-Pet | pASK-IBA33plus expressing de novo synthesised | This study |
| | Pet, Amp^{r} | |
| pBADPet* | Derivative of pBADPet expressing de novo | Leyton et al, |
| | synthesised non cleaved Pet mutant (Pet*) with | 2011 |
| | N1018G and D1115G substitutions | |
| pASK-Pet* | pASK-Pet expressing Pet*, the non cleaved Pet | This study |
| | mutant | |
| pASK-His₆-Pet | pASK-Pet encoding Pet with a His₆-tag incorporated | This study |
| | after signal sequence | |
| pASK-His₆-Pet-ΔD1 | Similar to pASK-His₆-Pet; encodes Pet deleted of | This study |
| | domain 1 | |
| pET-Pet | pET22b expressing de novo synthesised Pet, Amp^{r} | This study |
| pMA-FS-ESAT6 | pMA cloning vector carrying de novo synthesized | GenScript/This |
| | esxA gene encoding ESAT-6 from Mycobacterium | study |
| | tuberculosis; Amp^{r} also carries a phagosomal | |
| | fusogenic sequence (FS) at the 5′ end of the gene, | |
| | Amp^{r} | |
| pMA-Ag85B | pMA cloning vector containing de novo synthesised | GenScript/This |
| | fbpB encoding a putative esterase, antigen 85-B from | study |
| | Mycobacterium tuberculosis, Amp^{r} | |
| pET-LatA | pET22b expressing de novo synthesised LatA (locus | GenScript/This |
| | LI0649), a putative surface protein from Lawsonia | study |
| | intracellularis, Amp^{r} | |
| pET-Pmp17 | pET22b containing de novo synthesized pmp17G | GenScript/This |
| | gene encoding polymorphic outer membrane protein | study |
| | Pmp17 from Chlamydophila abortus, Amp^{r} | |
| pET-SapA | pET22b containing de novo synthesized yaiT gene | GenArt/This |
| | encoding putative surface protein SapA from | study |
| | Salmonella enterica subsp. Enterica serovar | |
| | Typhimurium, Amp^{r} | |
| pET-YapA | pET22b containing de novo synthesized yapA gene | Epoch Life |
| | encoding putative surface protein YapA from Yersinia | Science/This |
| | pestis, Amp^{r} | study |
| pET-BMAA1263 | pET22b containing de novo synthesised BMAA1263, | Epoch Life |
| | a putative surface protein from Burkholderia mallei, | Science/This |
| | Amp^{r} | study |
| pUC74-mCherry | pUC74 cloning vector carrying de novo synthesised | GenScript/This |
| | red fluorescent protein mCherry, Amp^{r} | study |
| pASK-ESAT6-Pet-BB | pASK-Pet with esxA insertion between BglII/BstBI | This study |
| | sites of pet | |
| pASK-ESAT6-Pet* | pASK-ESAT6-Pet-BB derivative expressing non | This study |
| | cleaved ESAT6-Pet* fusion containing N1018G and | |
| | D1115G substitutions in Pet secretion unit | |
| pASK-ESAT6-Pet-BP | pASK-Pet with esxA insertion between BglII/PstI sites | This study |
| | of pet | |
| pASK-FS-ESAT6-Pet- | Similar to pASK-ESAT6-Pet-BP but expresses | This study |
| BP | ESAT-6 with N-terminal FS sequence | |
| pASK-Ag85B-Pet-BB | pASK-Pet with fbpB insertion between BglII/BstBI | This study |
| | sites of pet | |
| pASK-Ag85B-Pet-BP | pASK-Pet with fbpB insertion between BglII/PstI sites | |
| | of pet | |
| pASK-Ag85B-ESAT6- | pASK-Ag85B-Pet-BB with esxA insertion between | This study |
| Pet | BstBI/PstI sites of pet | |
| pASK-Pmp17-Pet-BB | pASK-Pet with insertion of the part of pmp17G gene | This study |
| | encoding predicted surface domain of Pmp17 | |
| | between BglII/BstBI sites of pet | |
| pASK-His₆-Pmp17- | pASK-Pmp17-Pet-BB with the His₆-tag engineered | This study |
| Pet-BB | after Pet signal sequence cleavage site | |
| pASK-SapA-Pet-BB | pASK-Pet with insertion of the part of yaiT gene | This study |
| | encoding predicted extracellular domain of SapA | |
| | between BglII/BstBI sites of pet | |
| pASK-SapA-Pet-BP | pASK-Pet with insertion of the part of yaiT gene | This study |
| | encoding predicted extracellular domain of SapA | |
| | between BglII/PstI sites of pet | |
| pASK-His₆-SapA-Pet- | pASK-SapA-Pet-BP with the His₆-tag engineered | This study |
| BP | after Pet signal sequence cleavage site | |
| pASK-YapA-Pet-BP | pASK-Pet with insertion of the part of yapA gene | This study |
| | encoding predicted extracellular domain of YapA | |
| | between BglII/PstI sites of pet | |
| pASK-His₆-YapA-Pet- | pASK-YapA-Pet-BP with the His₆-tag engineered | This study |
| BP | after Pet signal sequence cleavage site | |
| pASK-LatA-Pet-BC | pASK-Pet carrying insertion latA gene fragment | This study |
| | encoding predicted extracellular domain of LatA | |
| | between BglII/ClaI sites of pet | |
| pASK-LatA-Pet-BP | pASK-Pet carrying insertion latA gene fragment | This study |
| | encoding predicted extracellular domain of LatA | |
| | between BglII/PstI sites of pet | |
| pASK-BMAA1263- | pASK-Pet carrying insertion of a predicted | This study |
| Pet-BP | extracellular domain of BMAA1263 between | |
| | BglII/PstI sites of pet | |
| pASK-mCherry-Pet- | pASK-Pet with mcherry insertion between BglII/PstI | This study |
| BP | sites of pet | |
| pASK-mCherry-Pet* | pASK-mCherry-Pet-BP derivative expressing non | This study |
| | cleaved mCherry-Pet* fusion containing N1018G and | |
| | D1115G substitutions in Pet secretion unit | |
| pASK-His₆-mCherry- | pASK-mCherry-Pet-BP with the His₆-tag engineered | This study |
| Pet-BP | after Pet signal sequence cleavage site | |
| pET-Prn-Pet | pET22b containing de novo synthesized prn-pet | GenScript/This |
| | sequence encoding extracellular domain of Pertactin | study |
| | (P69.C) from Bordetella pertussis | |
| Pet secretion unit | | |
| pASK-ESAT6-Pet Δ*1 | pASK-ESAT6-Pet-BP derivative containing truncated | This study |
| | 3′ pet gene fragment corresponding to Pet 840-1295 | |
| | protein sequence | |
| pASK-ESAT6-Pet Δ*2 | As above, contains pet fragment encoding Pet 889-1295 | This study |
| pASK-ESAT6-Pet Δ*3 | As above, contains pet fragment encoding Pet 925-1295 | This study |
| pASK-ESAT6-Pet Δ*4 | As above, contains pet fragment encoding Pet 936-1295 | This study |
| pASK-ESAT6-Pet Δ*5 | As above, contains pet fragment encoding Pet 947-1295 | This study |
| pASK-ESAT6-Pet Δ*6 | As above, contains pet fragment encoding Pet 958-1295 | This study |
| pASK-ESAT6-Pet Δ*7 | As above, contains pet fragment encoding Pet 960-1295 | This study |
| pASK-ESAT6-Pet Δ*8 | As above, contains pet fragment encoding Pet 962-1295 | This study |
| pASK-ESAT6-Pet Δ*9 | As above, contains pet fragment encoding Pet 964-1295 | This study |
| pASK-ESAT6-Pet | As above, contains pet fragment encoding Pet 966-1295 | This study |
| Δ*10 | | |
| pASK-ESAT6-Pet | As above, contains pet fragment encoding Pet 968-1295 | This study |
| Δ*11 | | |
| pASK-ESAT6-Pet | As above, contains pet fragment encoding Pet 971-1295 | This study |
| Δ*12 | | |
| pASK-ESAT6-Pet | As above, contains pet fragment encoding Pet 975-1295 | This study |
| Δ*13 | | |
| pASK-ESAT6-Pet | As above, contains pet fragment encoding Pet 979-1295 | This study |
| Δ*14 | | |
| pASK-ESAT6-Pet | As above, contains pet fragment encoding Pet 982-1295 | This study |
| Δ*15 | | |
| pASK-ESAT6-Pet | As above, contains pet fragment encoding Pet 985-1295 | This study |
| Δ*16 | | |
| pASK-ESAT6-Pet | As above, contains pet fragment encoding Pet 988-1295 | This study |
| Δ*17 | | |
| pASK-ESAT6-Pet | As above, contains pet fragment encoding Pet 994-1295 | This study |
| Δ*18 | | |
| pASK-ESAT6-Pet | As above, contains pet fragment encoding Pet 1002-1295 | This study |
| Δ*19 | | |
| pASK-ESAT6-Pet | As above, contains pet fragment encoding Pet 1010-1295 | This study |
| Δ*20 | | |
| Pet AC mutants | | |
| pASK-ESAT6-Pet Δ*6 | pASK-ESAT6-Pet Δ*6 derivative carrying L987→A | This study |
| L987A | substitution in AC domain | |
| pASK-ESAT6-Pet Δ*6 | pASK-ESAT6-Pet Δ*6 derivative carrying L987→K | This study |
| L987K | substitution in AC domain | |
| pASK-ESAT6-Pet Δ*6 | pASK-ESAT6-Pet Δ*6 derivative carrying G989→A | This study |
| G989A | substitution in AC domain | |
| pASK-ESAT6-Pet Δ*6 | pASK-ESAT6-Pet Δ*6 derivative carrying G989→K | This study |
| G989K | substitution in AC domain | |
| pASK-ESAT6-Pet Δ*6 | pASK-ESAT6-Pet Δ*6 derivative carrying W985→A | This study |
| W985A | substitution in AC domain | |
| pASK-ESAT6-Pet Δ*6 | pASK-ESAT6-Pet Δ*6 derivative carrying W985→K | This study |
| W985K | substitution in AC domain | |
| pASK-ESAT6-Pet Δ*6 | pASK-ESAT6-Pet Δ*6 derivative carrying I974→A | This study |
| I974A | substitution in AC domain | |
| pASK-ESAT6-Pet Δ*6 | pASK-ESAT6-Pet Δ*6 derivative carrying I974→K | This study |
| I974K | substitution in AC domain | |
| Pic secretion unit | | |
| pASK-ESAT6-Pic Δ*6 | pASK-ESAT6-Pet Δ*6 in which 3′ PstI-HindIII | This study |
| | fragment of pet gene was replaced with the | |
| | equivalent fragment from pic gene corresponding to | |
| | Pic 1035-1372 protein sequence | |
| pASK-ESAT6-Pic | pASK-ESAT6-Pet Δ*12 in which 3′ PstI-HindIII | This study |
| Δ*12 | fragment of pet gene was replaced with the | |
| | equivalent fragment from pic gene corresponding to | |
| | Pic 1048-1372 protein sequence | |
| pASK-ESAT6-Pic | pASK-ESAT6-Pet Δ*17 in which 3′ PstI-HindIII | This study |
| Δ*17 | fragment of pet gene was replaced with the | |
| | equivalent fragment from pic gene corresponding to | |
| | Pic 1065-1372 protein sequence | |
| pASK-ESAT6-Pic | pASK-ESAT6-Pet Δ*19 in which 3′ PstI-HindIII | This study |
| Δ*19 | fragment of pet gene was replaced with the | |
| | equivalent fragment from pic gene corresponding to | |
| | Pic 1079-1372 protein sequence | |
| pASK-ESAT6-Pic | pASK-ESAT6-Pet Δ*20 in which 3′ PstI-HindIII | This study |
| Δ*20 | fragment of pet gene was replaced with the | |
| | equivalent fragment from pic gene corresponding to | |
| | Pic 1087-1372 protein sequence | |

TABLE 4
Primers used in this study

| | SEQ | |
|---|---|---|
| Primer | ID NO. | Sequence (5′-3′)* |
| BsaI-pet-F | 33 | ATGGTAGGTCTCAAATGAACAAAATCTACTCTATC |
| HindIII-pet-R | 34 | GCGCAAGCTTTTATCAATGATGATGATGATGATGACC |
| SacI-pet-F | 35 | TTTCTGAGCTCGCCAAAAAAGTTATCTGC |
| PetSS-BgIII-AfIII- | 36 | TTCGAACTTAAGAGATCTAGGTGATGGTGATGGTGATGCGC |
| BstBI-R | | CGCGTAGATGATGTTGGTGTAAG |
| BgIII-ESAT6-F | 37 | GCGAGATCTGATGACCGAACAGCAGTGGAAC |
| BgIII-FS-ESAT6-F | 38 | TTATAGATCTGATGGAAGCTGCTGCTGC |
| BstBI-ESAT6-F | 39 | GGCGTTCGAAAATGACCGAACAGCAGTGGAAC |
| PstI-ESAT6-R | 40 | ATCCTGCAGAGCCGCCAGCGAACATGC |
| BstBI-ESAT6-R | 41 | TATATTCGAATCGCCGCCAGCGAACATG |
| BgIII-Ag85B-F | 42 | CCAAGATCTGATGACCGACGTTTCTCGTAA |
| BstBI-Ag85B-R | 43 | TTCCTTCGAATCGCCGCCACCAGCACCCAG |
| PstI-Ag85B-R | 44 | TAACTGCAGAGCCGCCACCAGCACCCAG |
| BgIII-LatA-F | 45 | TGTTAGATCTGGAAGCGGTTGAACACTTCG |
| Clal-LatA-R | 46 | TATGATCGATGTTCGCGATGATGTGGTTG |
| PstI-LatA-R | 47 | TATACTGCAGAGTTCGCGATGATGTGGTTG |
| BgIII-Pmp17-F | 48 | AATAAGATCTGAACGACGCGCAGACCGC |
| BstBI-Pmp17-R | 49 | TATATTCGAATCCGCCAGTTTCGCCGGAG |
| BgIII-SapA-F | 50 | GCCGAGATCTGACCACCTATGATACCTGGACC |
| BstBI-SapA-R | 51 | CCGTTTCGAATCGCCATCGCTGTTCATCGCAATG |
| PstI-SapA-R | 52 | CCGGCTGCAGAGCCATCGCTGTTCATCGCAATG |
| BgIII-YapA-F | 53 | ACGTAGATCTGGTTTCTCAGATCGCGACCACCG |
| PstI-YapA-R | 54 | TAGCCTGCAGACGCGTTAGACATGTCAACGGTACC |
| BgIII-BMAA1263-F | 55 | ACGTAGATCTGGCGCCGTACCCGGACCCG |
| PstI-BMAA1263-R | 56 | TAGCCTGCAGAACCACCCGCCGCGTTCAGGATC |
| BgIII-mCherry-F | 57 | GCCGAGATCTGATGGTTTCTAAAGGTGAAGAAGAC |
| PstI-mCherry-R | 58 | CCGTCTGCAGATTTATACAGTTCGTCCATACCGC |
| PstI-TSYQ-del1-F | 59 | TATGCTGCAGACACCTCTTACCAGGGTTCTATCAAAGC |
| PstI-TLTV-del2-F | 60 | TATACTGCAGACACCCTGACCGTTGACGAACTGACC |
| PstI-NLLL-del3-F | 61 | TATACTGCAGACAACCTGCTGCTGGTCGACTTCATCG |
| PstI-TPEI-del12-F | 62 | TATACTGCAGACACCCCGGAAATCAAACAGCAGG |
| PstI-YKAF-del20-F | 63 | TATGCTGCAGACTACAAAGCGTTCCTGGCGGAAG |
| PstI-GNDK-del4-F | 64 | TATGCTGCAGACGGTAACGACAAAAACGGTCTGAAC |
| PstI-VKAP-del5-F | 65 | TATACTGCAGACGTTAAAGCGCCGGAAAACACCTC |
| PstI-FKTE-del6-F | 66 | TATGCTGCAGACTTCAAAACCGAAACCCAGACCATC |
| PstI-TGYK-del17-F | 67 | TATCCTGCAGACACCGGCTACAAAACCGTTGCG |
| PstI-TETQ-del7-F | 68 | TATACTGCAGACACCGAAACCCAGACCATCGG |
| PstI-TQTI-del8-F | 69 | TATACTGCAGACACCCAGACCATCGGTTTCTCTG |
| PstI-TIGF-del9-F | 70 | CATACTGCAGACACCATCGGTTTCTCTGACGTTACC |
| PstI-GFSD-del10-F | 71 | TGTACTGCAGACGGTTTCTCTGACGTTACCCCG |
| PstI-SDVT-del11-F | 72 | TCTGCTGCAGACTCTGACGTTACCCCGGAAATC |
| PstI-KQQE-del13-F | 73 | GGCACTGCAGACAAACAGCAGGAAAAAGACGGTAAATC |
| PstI-KDG-del14-F | 74 | GGCACTGCAGACAAAGACGGTAAATCTGTTTGGACC |
| PstI-KSV-del15-F | 75 | GGCACTGCAGACAAATCTGTTTGGACCCTGACC |
| PstI-WTL-del16-F | 76 | TATACTGCAGACTGGACCCTGACCGGCTACAAAACC |
| PstI-ANAD-del18-F | 77 | GGCACTGCAGACGCGAACGCGGACGCGGCG |
| PstI-ATSL-del19-F | 78 | GGCACTGCAGACGCGACCTCTCTGATGTCTGGTGG |
| SbfI-FKAG-Pic- | 79 | GGCACCTGCAGGCTTTAAGGCCGGCACCCGGGTGAC |
| del6-F | | |
| SbfI-TPTL-Pic- | 80 | GGCACCTGCAGGCACCCCAACCCTGCATGTTGATACC |
| del12-F | | |
| SbfI-DGFK-Pic- | 81 | GGCACCTGCAGGCGATGGTTTTAAAGCGGAGGCTGATAAAG |
| del17-F | | |
| SbfI-ADSF-Pic- | 82 | GGCACCTGCAGGCGCTGACAGTTTCATGAATGCCGGG |
| del19-F | | |
| SbfI-YKNF-Pic- | 83 | GGCACCTGCAGGCTATAAAAACTTCATGACGGAAGTTAAC |
| del20-F | | |
| HindIII-Pic-end-R | 84 | GCGCAAGCTTTCAGAACATATACCGGAAATTCGCG |
| pASK-IBA33+ | 85 | GAGTTATTTTACCACTCCCT |
| Forward seq | | |
| pASK-IBA33+ | 86 | CGCAGTAGCGGTAAACG |
| Reverse seq | | |

Site directed mutagenesis primers

| | | |
|---|---|---|
| Bow tie_Ile to | 87 | GACGTTACCCCGGAAGCGAAACAGCAGGAAAAAG |
| Ala_F | | |
| Bow tie_Ile to | 88 | CTTTTTCCTGCTGTTTCGCTTCCGGGGTAACGTC |
| Ala_R | | |
| Bow tie_Ile to | 89 | GACGTTACCCCGGAAAAAAAACAGCAGGAAAAAG |
| Lys_F | | |
| Bow tie_Ile to | 90 | CTTTTTCCTGCTGTTTTTTTTCCGGGGTAACGTC |
| Lys_R | | |
| Bow tie_W to | 91 | AAAGACGGTAAATCTGTTGCGACCCTGACCGGCTAC |
| Ala_F | | |
| Bow tie_W to | 92 | GTAGCCGGTCAGGGTCGCAACAGATTTACCGTCTTT |
| Ala_R | | |
| Bow tie_W to | 93 | AAAGACGGTAAATCTGTTAAAACCCTGACCGGCTAC |
| Lys_F | | |
| Bow tie_W to | 94 | GTAGCCGGTCAGGGTTTTAACAGATTTACCGTCTTT |
| Lys_R | | |
| Bow tie_Leu to | 95 | GTAAATCTGTTTGGACCGCGACCGGCTACAAAACC |
| Ala_F | | |
| Bow tie_Leu to | 96 | GGTTTTGTAGCCGGTCGCGGTCCAAACAGATTTAC |
| Ala_R | | |
| Bow tie_Leu to | 97 | GTAAATCTGTTTGGACCAAAACCGGCTACAAAACC |
| Lys_F | | |
| Bow tie_Leu to | 98 | GGTTTTGTAGCCGGTTTTGGTCCAAACAGATTTAC |
| Lys_R | | |
| Bow tie_Gly to | 99 | CTGTTTGGACCCTGACCGCGTACAAAACCGTTGCG |
| Ala_F | | |
| Bow tie_Gly to | 100 | CGCAACGGTTTTGTACGCGGTCAGGGTCCAAACAG |
| Ala_R | | |
| Bow tie_Gly to | 101 | CTGTTTGGACCCTGACCAAATACAAAACCGTTGCG |
| Lys_F | | |
| Bow tie_Gly to | 102 | CGCAACGGTTTTGTATTTGGTCAGGGTCCAAACAG |
| Lys_R | | |

*Restriction sites are in bold font, mutant codons are underlined

### Amino Acid and Nucleic Acid Sequences Discussed in the Application

SEQ ID NO: 1 - PET_ECO44 amino acid sequence
MNKIYSIKYSAATGGLIAVSELAKKVICKTNRKISAALLSLAVISYTNIIYAANMDISKAWARDYLDLAQ
NKGVFQPGSTHVKIKLKDGTDFSFPALPVPDFSSATANGAATSIGGAYAVTVAHNAKNKSSANYQTYGST
QYTQINRMTTGNDFSIQRLNKYVVETRGADTSFNYNENNQNIIDRYGVDVGNGKKEIIGFRVGSGNTTFS
GIKTSQTYQADLLSASLFHITNLRANTVGGNKVEYENDSYFTNLTTNGDSGSGVYVFDNKEDKWVLLGTT
HGIIGNGKTQKTYVTPFDSKTTNELKQLFIQNVNIDNNTATIGGGKITIGNTTQDIEKNKNNQNKDLVFS
GGGKISLKENLDLGYGGFIFDENKKYTVSAEGNNNVTFKGAGIDIGKGSTVDWNIKYASNDALHKIGEGS
LNVIQAQNTNLKTGNGTVILGAQKTFNNIYVAGGPGTVQLNAENALGEGDYAGIFFTENGGKLDLNGHNQ
TFKKIAATDSGTTITNSNTTKESVLSVNNQNNYIYHGNVDGNVRLEHHLDTKQDNARLILDGDIQANSIS
IKNAPLVMQGHATDHAIFRTTKTNNCPEFLCGVDWVTRIKNAENSVNQKNKTTYKSNNQVSDLSQPDWET
RKFRFDNLNIEDSSLSIARNADVEGNIQAKNSVINIGDKTAYIDLYSGKNITGAGFTFRQDIKSGDSIGE
SKFTGGIMATDGSISIGDKAIVTLNTVSSLDRTALTIHKGANVTASSSLFTTSNIKSGGDLTLTGATEST
GEITPSMFYAAGGYELTEDGANFTAKNQASVTGDIKSEKAAKLSFGSADKDNSATRYSQFALAMLDGFDT
SYQGSIKAAQSSLAMNNALWKVTGNSELKKLNSTGSMVLFNGGKNIFNTLTVDELTTSNSAFVMRTNTQQ
ADQLIVKNKLEGANNLLLVDFIEKKGNDKNGLNIDLVKAPENTSKDVFKTETQTIGFSDVTPEIKQQEKD
GKSVWTLTGYKTVANADAAKKATSLMSGGYKAFLAEVNNLNKRMGDLRDINGEAGAWARIMSGTGSAGGG
FSDNYTHVQVGADNKHELDGLDLFTGVTMTYTDSHAGSDAFSGETKSVGAGLYASAMFESGAYIDLIGKY
VHHDNEYTATFAGLGTRDYSSHSWYAGAEVGYRYHVTDSAWIEPQAELVYGAVSGKQFSWKDQGMNLTMK
DKDFNPLIGRTGVDVGKSFSGKDWKVTARAGLGYQFDLFANGETVLRDASGEKRIKGEKDGRMLMNVGLN
AEIRDNVRFGLEFEKSAFGKYNVDNAINANFRYSF
SEQ ID NO: 2 Secretion unit from PET_ECO44
YKAFLAEVNNLNKRMGDLRDINGEAGAWARIMSGTGSAGGGFSDNYTHVQVGADNKHELDGLDLFTGVTM
TYTDSHAGSDAFSGETKSVGAGLYASAMFESGAYIDLIGKYVHHDNEYTATFAGLGTRDYSSHSWYAGAE
VGYRYHVTDSAWIEPQAELVYGAVSGKQFSWKDQGMNLTMKDKDFNPLIGRTGVDVGKSFSGKDWKVTAR
AGLGYQFDLFANGETVLRDASGEKRIKGEKDGRMLMNVGLNAEIRDNVRFGLEFEKSAFGKYNVDNAINA
NFRYSF
SEQ ID NO: 3 PET_ECO44 nucleic acid sequence encoding secretion unit
TATAAAGCCT TCCTTGCAGA GGTCAACAAC CTCAACAAAC GTATGGGTGA TCTGCGTGAC 60
ATTAACGGTG AGGCCGGTGC ATGGGCCCGT ATCATGAGTG GAACCGGGTC TGCCGGCGGT 120
GGATTCAGTG ACAACTACAC CCACGTTCAG GTCGGTGCGG ATAACAAACA TGAACTCGAT 180
GGCCTTGACC TCTTCACCGG GGTGACCATG ACCTATACCG ACAGCCATGC AGGCAGTGAT 240
GCCTTCAGTG GTGAAACGAA GTCTGTGGGT GCCGGTCTCT ATGCCTCTGC CATGTTTGAG 300
TCCGGAGCAT ATATCGACCT CATCGGTAAG TACGTTCACC ATGACAACGA GTATACCGCA 360
ACTTTCGCCG GCCTTGGCAC CAGAGACTAC AGCTCCCACT CCTGGTATGC CGGTGCGGAA 420
GTCGGTTACC GTTACCATGT AACTGACTCT GCATGGATTG AGCCGCAGGC GGAACTTGTT 480
TACGGTGCTG TATCCGGGAA ACAGTTCTCC TGGAAGGACC AGGGAATGAA CCTCACCATG 540
AAGGATAAGG ACTTTAATCC GCTGATTGGG CGTACCGGTG TTGATGTGGG TAAATCCTTC 600
TCCGGTAAGG ACTGGAAAGT CACAGCCCGC GCCGGCCTTG GCTACCAGTT TGACCTGTTT 660
GCCAACGGTG AAACTGTACT GCGTGATGCG TCCGGTGAAA AACGTATCAA AGGTGAAAAA 720
GACGGCCGTA TGCTCATGAA TGTTGGTCTG AATGCTGAGA TTCGTGACAA CGTACGCTTT 780
GGTCTTGAGT TTGAGAAATC GGCATTTGGT AAGTACAACG TGGATAACGC CATCAACGCC 840
AACTTCCGTT ACTCCTTCTG A
SEQ ID NO: 4 - SAT_CFT073 amino acid sequence
MREYMNKIYSLKYSAATGGLIAVSELAKRVSGKTNRKLVATMLSLAVAGTVNAANIDISNVWARDYLDLA
QNKGIFQPGATDVTITLKNGDKFSFHNLSIPDFSGAAASGAATAIGGSYSVTVAHNKKNPQAAETQVYAQ
SSYRVVDRRNSNDFEIQRLNKFVVETVGATPAETNPTTYSDALERYGIVTSDGSKKIIGFRAGSGGTSFI
NGESKISTNSAYSHDLLSASLFEVTQWDSYGMMIYKNDKTFRNLEIFGDSGSGAYLYDNKLEKWVLVGTT
HGIASVNGDQLTWITKYNDKLVSELKDTYSHKINLNGNNVTIKNTDITLHQNNADTTGTQEKITKDKDIV
FTNGGDVLFKDNLDFGSGGIIFDEGHEYNINGQGFTFKGAGIDIGKESIVNWNALYSSDDVLHKIGPGTL
NVQKKQGANIKIGEGNVILNEEGTFNNIYLASGNGKVILNKDNSLGNDQYAGIFFTKRGGTLDLNGHNQT
FTRIAATDDGTTITNSDTTKEAVLAINNEDSYIYHGNINGNIKLTHNINSQDKKTNAKLILDGSVNTKND
VEVSNASLTMQGHATEHAIFRSSANHCSLVFLCGTDWVTVLKETESSYNKKFNSDYKSNNQQTSFDQPDW
KTGVFKFDTLHLNNADFSISRNANVEGNISANKSAITIGDKNVYIDNLAGKNITNNGFDFKQTISTNLSI
GETKFTGGITAHNSQIAIGDQAVVTLNGATFLDNTPISIDKGAKVIAQNSMFTTKGIDISGELTMMGIPE
QNSKTVTPGLHYAADGFRLSGGNANFIARNMASVTGNIYADDAATITLGQPETETPTISSAYQAWAETLL
YGFDTAYRGAITAPKATVSMNNAIWHLNSQSSINRLETKDSMVRFTGDNGKFTTLTVNNLTIDDSAFVLR
ANLAQADQLVVNKSLSGKNNLLLVDFIEKNGNSNGLNIDLVSAPKGTAVDVFKATTRSIGFSDVTPVIEQ
KNDTDKATWTLIGYKSVANADAAKKATLLMSGGYKAFLAEVNNLNKRMGDLRDINGESGAWARIISGTGS
AGGGFSDNYTHVQVGADNKHELDGLDLFTGVTMTYTDSHAGSDAFSGETKSVGAGLYASAMFESGAYIDL
IGKYVHHDNEYTATFAGLGTRDYSSHSWYAGAEVGYRYHVTDSAWIEPQAELVYGAVSGKQFSWKDQGMN
LTMKDKDFNPLIGRTGVDVGKSFSGKDWKVTARAGLGYQFDLFANGETVLRDASGEKRIKGEKDGRMLMN
VGLNAEIRDNLRFGLEFEKSAFGKYNVDNAINANFRYSF
SEQ ID NO: 5 - Secretion unit from SATCFT073
YKAFLAEVNNLNKRMGDLRDINGESGAWARIISGTGSAGGGFSDNYTHVQVGADNKHELDGLDLFTGVTM
TYTDSHAGSDAFSGETKSVGAGLYASAMFESGAYIDLIGKYVHHDNEYTATFAGLGTRDYSSHSWYAGAE
VGYRYHVTDSAWIEPQAELVYGAVSGKQFSWKDQGMNLTMKDKDFNPLIGRTGVDVGKSFSGKDWKVTAR
AGLGYQFDLFANGETVLRDASGEKRIKGEKDGRMLMNVGLNAEIRDNLRFGLEFEKSAFGKYNVDNAINA
NFRYSF
SEQ ID NO: 6 - SAT_CFT073 nucleic acid sequence encoding secretion unit
TATAAAGCCT TCCTTGCTGA GGTCAACAAC CTTAACAAAC GTATGGGTGA TCTGCGTGAC 60
ATTAACGGTG AGTCCGGTGC ATGGGCCCGA ATCATTAGCG GAACCGGGTC TGCCGGCGGT 120
GGATTCAGTG ACAACTACAC CCACGTTCAG GTCGGTGCGG ATAACAAACA TGAACTCGAT 180
GGCCTTGACC TCTTCACCGG GGTGACCATG ACCTATACCG ACAGCCATGC AGGCAGTGAT 240
GCCTTCAGTG GTGAAACGAA GTCTGTGGGT GCCGGTCTCT ATGCCTCTGC CATGTTTGAG 300
TCCGGAGCAT ATATCGACCT CATCGGTAAG TACGTTCACC ATGACAACGA GTATACCGCA 360
ACTTTCGCCG GCCTTGGCAC CAGAGACTAC AGCTCCCACT CCTGGTATGC CGGTGCGGAA 420
GTCGGTTACC GTTACCATGT AACTGACTCT GCATGGATTG AGCCGCAGGC GGAACTTGTT 480
TACGGTGCTG TATCCGGGAA ACAGTTCTCC TGGAAGGACC AGGGAATGAA CCTCACCATG 540
AAGGATAAGG ACTTTAATCC GCTGATTGGG CGTACCGGTG TTGATGTGGG TAAATCCTTC 600
TCCGGTAAGG ACTGGAAAGT CACAGCCCGC GCCGGCCTTG GCTACCAGTT TGACCTGTTT 660
GCCAACGGTG AAACCGTACT GCGTGATGCG TCCGGTGAGA AACGTATCAA AGGTGAAAAA 720
GACGGTCGTA TGCTCATGAA TGTTGGTCTC AACGCCGAAA TTCGCGATAA TCTTCGCTTC 780
GGTCTTGAGT TTGAGAAATC GGCATTTGGT AAATACAACG TGGATAACGC GATCAACGCC 840
AACTTCCGTT ACTCTTTCTG A
SEQ ID NO: 7 - ESPP_ECO57 amino acid sequence
MNKIYSLKYSHITGGLIAVSELSGRVSSRATGKKKHKRILALCFLGLLQSSYSFASQMDISNFYIRDYMD
FAQNKGIFQAGATNIEIVKKDGSTLKLPEVPFPDFSPVANKGSTTSIGGAYSITATHNTKNHHSVATQNW
GNSTYKQTDWNTSHPDFAVSRLDKFVVETRGATEGADISLSKQQALERYGVNYKGEKKLIAFRAGSGVVS
VKKNGRITPFNEVSYKPEMLNGSFVHIDDWSGWLILTNNQFDEFNNIASQGDSGSALFVYDNQKKKWVVA
GTVWGIYNYANGKNHAAYSKWNQTTIDNLKNKYSYNVDMSGAQVATIENGKLTGTGSDTTDIKNKDLIFT
GGGDILLKSSFDNGAGGLVFNDKKTYRVNGDDFTFKGAGVDTRNGSTVEWNIRYDNKDNLHKIGDGTLDV
RKTQNTNLKTGEGLVILGAEKTFNNIYITSGDGTVRLNAENALSGGEYNGIFFAKNGGTLDLNGYNQSFN
KIAATDSGAVITNTSTKKSILSLNNTADYIYHGNINGNLDVLQHHETKKENRRLILDGGVDTTNDISLRN
TQLSMQGHATEHAIYRDGAFSCSLPAPMRFLCGSDYVAGMQNTEADAVKQNGNAYKTNNAVSDLSQPDWE
TGTFRFGTLHLENSDFSVGRNANVIGDIQASKSNITIGDTTAYIDLHAGKNITGDGFGFRQNIVRGNSQG
ETLFTGGITAEDSTIVIKDKAKALFSNYVYLLNTKATIENGADVTTQSGMFSTSDISISGNLSMTGNPDK
DNKFEPSIYLNDASYLLTDDSARLVAKNKASVVGDIHSTKSASIMFGHDESDLSQLSDRTSKGLALGLLG
GFDVSYRGSVNAPSASATMNNTWWQLTGDSALKTLKSTNSMVYFTDSANNKKFHTLTVDELATSNSAYAM
RTNLSESDKLEVKKHLSGENNILLVDFLQKPTPEKQLNIELVSAPKDTNENVFKASKQTIGFSDVTPVIT
TRETDDKITWSLTGYNTVANKEATRNAAALFSVDYKAFLNEVNNLNKRMGDLRDINGEAGAWARIMSGTG
SASGGFSDNYTHVQVGVDKKHELDGLDLFTGFTVTHTDSSASADVFSGKTKSVGAGLYASAMFDSGAYID
LIGKYVHHDNEYTATFAGLGTRDYSTHSWYAGAEAGYRYHVTEDAWIEPQAELVYGSVSGKQFAWKDQGM
HLSMKDKDYNPLIGRTGVDVGKSFSGKDWKVTARAGLGYQFDLLANGETVLRDASGEKRIKGEKDSRMLM
SVGLNAEIRDNVRFGLEFEKSAFGKYNVDNAVNANFRYSF
SEQ ID NO: 8 - Secretion unit from ESPP_ECO57
YKAFLNEVNNLNKRMGDLRDINGEAGAWARIMSGTGSASGGFSDNYTHVQVGVDKKHELDGLDLFTGFTV
THTDSSASADVFSGKTKSVGAGLYASAMFDSGAYIDLIGKYVHHDNEYTATFAGLGTRDYSTHSWYAGAE
AGYRYHVTEDAWIEPQAELVYGSVSGKQFAWKDQGMHLSMKDKDYNPLIGRTGVDVGKSFSGKDWKVTAR
AGLGYQFDLLANGETVLRDASGEKRIKGEKDSRMLMSVGLNAEIRDNVRFGLEFEKSAFGKYNVDNAVNA
NFRYSF
SEQ ID NO: 9 - ESPP_ECO57 nucleic acid sequence encoding secretion unit
TATAAAAATT TTCTTGCTGA AGTCAACAAC CTGAACAAAC GTATGGGTGA CCTGCGTGAC 60
ATCAACGGCG AAGCCGGTGC ATGGGCACGC ATCATGAGCG GTACCGGCTC TGCCAGTGGT 120
GGTTTCAGTG ACAACTACAC GCACGTTCAG GTCGGGGTCG ACAAAAAACA TGAGCTGGAC 180
GGACTGGATT TGTTTACCGG TTTCACTGTC ACACACACTG ACAGCAGTGC CTCCGCCGAT 240
GTTTTCAGCG GTAAAACGAA GTCTGTGGGG GCTGGCCTGT ATGCTTCCGC CATGTTTGAT 300
TCCGGTGCCT ATATCGACCT GATTGGCAAG TATGTTCACC ATGATAATGA GTACACAGCA 360
ACCTTTGCCG GACTCGGAAC CCGTGATTAC AGCACGCATT CATGGTATGC CGGTGCAGAA 420
GCGGGCTACC GCTATCATGT CACTGAGGAT GCCTGGATTG AGCCACAGGC TGAGCTGGTT 480
TACGGTTCTG TATCCGGTAA ACAGTTTGCA TGGAAGGACC AGGGAATGCA TCTGTCCATG 540
AAGGACAAGG ACTACAATCC GCTGATTGGC CGAACCGGTG TAGATGTGGG TAAATCCTTC 600
TCTGGTAAGG ACTGGAAAGT GACAGCCCGG GCCGGCCTGG GCTACCAGTT CGACCTGCTG 660
GCTAACGGCG AAACCGTATT GCGGGATGCA TCTGGTGAAA AACGCATCAA AGGTGAAAAG 720
GACAGCCGTA TGCTGATGTC CGTTGGCCTG AATGCAGAAA TCAGGGACAA CGTCCGCTTT 780
GGACTGGAGT TTGAGAAATC CGCCTTTGGT AAGTACAACG TTGATAATGC AGTCAACGCT 840
AACTTCCGTT ACTCGTTCTG A
SEQ ID NO: 10 - SIGA_SHIFL amino acid sequence
MNKIYSLKYSHITGGLVAVSELTRKVSVGTSRKKVILGIILSSIYGSYGETAFAAMLDINNIWTRDYLDL
AQNRGEFRPGATNVQLMMKDGKIFHFPELPVPDFSAVSNKGATTSIGGAYSVTATHNGTQHHAITTQSWD
QTAYKASNRVSSGDFSVHRLNKFVVETTGVTESADFSLSPEDAMKRYGVNYNGKEQIIGFRAGAGTTSTI
LNGKQYLFGQNYNPDLLSASLFNLDWKNKSYIYTNRTPFKNSPIFGDSGSGSYLYDKEQQKWVFHGVTST
VGFISSTNIAWTNYSLFNNILVNNLKKNFTNTMQLDGKKQELSSIIKDKDLSVSGGGVLTLKQDTDLGIG
GLIFDKNQTYKVYGKDKSYKGAGIDIDNNTTVEWNVKGVAGDNLHKIGSGTLDVKIAQGNNLKIGNGTVI
LSAEKAFNKIYMAGGKGTVKINAKDALSESGNGEIYFTRNGGTLDLNGYDQSFQKIAATDAGTTVTNSNV
KQSTLSLTNTDAYMYHGNVSGNISINHIINTTQQHNNNANLIFDGSVDIKNDISVRNAQLTLQGHATEHA
IFKEGNNNCPIPFLCQKDYSAAIKDQESTVNKRYNTEYKSNNQIASFSQPDWESRKFNFRKLNLENATLS
IGRDANVKGHIEAKNSQIVLGNKTAYIDMFSGRNITGEGFGFRQQLRSGDSAGESSFNGSLSAQNSKITV
GDKSTVTMTGALSLINTDLIINKGATVTAQGKMYVDKAIELAGTLTLTGTPTENNKYSPAIYMSDGYNMT
EDGATLKAQNYAWVNGNIKSDKKASILFGVDQYKEDNLDKTTHTPLATGLLGGFDTSYTGGIDAPAASAS
MYNTLWRVNGQSALQSLKTRDSLLLFSNIENSGFHTVTVNTLDATNTAVIMRADLSQSVNQSDKLIVKNQ
LTGSNNSLSVDIQKVGNNNSGLNVDLITAPKGSNKEIFKASTQAIGFSNISPVISTKEDQEHTTWTLTGY
KVAENTASSGAAKSYMSGNYKAFLTEVNNLNKRMGDLRDTNGEAGAWARIMSGAGSASGGYSDNYTHVQI
GVDKKHELDGLDLFTGLTMTYTDSHASSNAFSGKTKSVGAGLYASAIFDSGAYIDLISKYVHHDNEYSAT
FAGLGTKDYSSHSLYVGAEAGYRYHVTEDSWIEPQAELVYGAVSGKRFDWQDRGMSVTMKDKDFNPLIGR
TGVDVGKSFSGKDWKVTARAGLGYQFDLFANGETVLRDASGEKRIKGEKDGRILMNVGLNAEIRDNLRFG
LEFEKSAFGKYNVDNAINANFRYSF
SEQ ID NO: 11 - Secretion unit from SIGA_SHIFL
YKAFLTEVNNLNKRMGDLRDTNGEAGAWARIMSGAGSASGGYSDNYTHVQIGVDKKHELDGLDLFTGLTM
TYTDSHASSNAFSGKTKSVGAGLYASAIFDSGAYIDLISKYVHHDNEYSATFAGLGTKDYSSHSLYVGAE
AGYRYHVTEDSWIEPQAELVYGAVSGKRFDWQDRGMSVTMKDKDFNPLIGRTGVDVGKSFSGKDWKVTAR
AGLGYQFDLFANGETVLRDASGEKRIKGEKDGRILMNVGLNAEIRDNLRFGLEFEKSAFGKYNVDNAINA
NFRYSF
SEQ ID NO: 12 - SIGA_SHIFL nucleic acid sequence encoding secretion unit
TACAAAGCCT TCCTGACAGA AGTCAACAAC CTGAATAAAC GAATGGGGGA TCTGCGTGAC 60
ACCAATGGCG AGGCCGGTGC ATGGGCCCGC ATCATGAGCG GAGCAGGTTC AGCTTCTGGT 120
GGATACAGTG ACAACTACAC CCATGTGCAG ATTGGTGTGG ATAAAAAACA TGAGCTGGAT 180
GGACTTGACC TTTTCACTGG TCTGACTATG ACGTATACCG ACAGTCATGC CAGCAGTAAT 240
GCATTCAGTG GCAAGACGAA GTCCGTCGGG GCAGGTCTGT ATGCTTCCGC TATATTTGAC 300
TCTGGTGCCT ATATCGACCT GATTAGTAAG TATGTTCACC ATGATAATGA GTACTCGGCG 360
ACCTTTGCTG GACTCGGAAC AAAAGACTAC AGTTCTCATT CCTTGTATGT GGGTGCTGAA 420
GCAGGCTACC GCTATCATGT AACAGAAGAC TCCTGGATTG AGCCGCAGGC AGAACTGGTT 480
TATGGGGCCG TATCAGGTAA ACGGTTCGAC TGGCAGGATC GCGGAATGAG CGTGACCATG 540
AAGGATAAGG ACTTTAATCC GCTGATTGGG CGTACCGGTG TTGATGTGGG TAAATCCTTC 600
TCCGGTAAGG ACTGGAAAGT CACAGCCCGC GCCGGCCTTG GCTACCAGTT TGACCTGTTT 660
GCCAACGGTG AAACCGTACT GCGTGATGCG TCCGGTGAGA AACGTATCAA AGGTGAAAAA 720
GACGGTCGTA TTCTCATGAA TGTTGGTCTC AACGCCGAAA TTCGCGATAA TCTTCGCTTC 780
GGTCTTGAGT TTGAGAAATC GGCATTTGGT AAATACAACG TGGATAACGC GATCAACGCC 840
AACTTCCGTT ACTCTTTCTG A
SEQ ID NO: 13 - ESPC_ECO27 amino acid sequence
MNKIYALKYCHATGGLIAVSELASRVMKKAARGSLLALFNLSLYGAFLSASQAAQLNIDNVWARDYLDLA
QNKGVFKAGATNVSIQLKNGQTFNFPNVPIPDFSPASNKGATTSIGGAYSVTATHNGTTHHAISTQNWGQ
SSYKYIDRMTNGDFAVTRLDKFVVETTGVKNSVDFSLNSHDALERYGVEINGEKKIIGFRVGAGTTYTVQ
NGNTYSTGQVYNPLLLSASMFQLNWDNKRPYNNTTPFYNETTGGDSGSGFYLYDNVKKEWVMLGTLFGIA
SSGADVWSILNQYDENTVNGLKNKFTQKVQLNNNTMSLNSDSFTLAGNNTAVEKNNNNYKDLSFSGGGSI
NFDNDVNIGSGGLIFDAGHHYTVTGNNKTFKGAGLDIGDNTTVDWNVKGVVGDNLHKIGAGTLNVNVSQG
NNLKTGDGLVVLNSANAFDNIYMASGHGVVKINHSAALNQNNDYRGIFFTENGGTLDLNGYDQSFNKIAA
TDIGALITNSAVQKAVLSVNNQSNYMYHGSVSGNTEINHQFDTQKNNSRLILDGNVDITNDINIKNSQLT
MQGHATSHAVFREGGVTCMLPGVICEKDYVSGIQQQENSANKNNNTDYKTNNQVSSFEQPDWENRLFKFK
TLNLINSDFIVGRNAIVVGDISANNSTLSLSGKDTKVHIDMYDGKNITGDGFGFRQDIKDGVSVSPESSS
YFGNVTLNNHSLLDIGNKFTGGIEAYDSSVSVTSQNAVFDRVGSFVNSSLTLEKGAKLTAQGGIFSTGAV
DVKENASLILTGTPSAQKQEYYSPVISTTEGINLGDKASLSVKNMGYLSSDIHAGTTAATINLGDGDAET
DSPLFSSLMKGYNAVLSGNITGEQSTVNMNNALWYSDGNSTIGTLKSTGGRVELGGGKDFATLRVKELNA
NNATFLMHTNNSQADQLNVTNKLLGSNNTVLVDFLNKPASEMNVTLITAPKGSDEKTFTAGTQQIGFSNV
TPVISTEKTDDATKWMLTGYQTVSDAGASKTATDFMASGYKSFLTEVNNLNKRMGDLRDTQGDAGVWARI
MNGTGSADGGYSDNYTHVQIGADRKHELDGVDLFTGALLTYTDSNASSHAFSGKTKSVGGGLYASALFDS
GAYFDLIGKYLHHDNQYTASFASLGTKDYSSHSWYAGAEVGYRYHLSEESWVEPQMELVYGSVSGKSFSW
EDRGMALSMKDKDYNPLIGRTGVDVGRTFSGDDWKITARAGLGYQFDLLANGETVLRDASGEKRFEGEKD
SRMLMNVGMNAEIKDNMRFGLELEKSAFGKYNVDNAINANFRYSF
SEQ ID NO: 14 - Secretion unit from ESPC_ECO27
YKSFLTEVNNLNKRMGDLRDTQGDAGVWARIMNGTGSADGGYSDNYTHVQIGADRKHELDGVDLFTGALL
TYTDSNASSHAFSGKTKSVGGGLYASALFDSGAYFDLIGKYLHHDNQYTASFASLGTKDYSSHSWYAGAE
VGYRYHLSEESWVEPQMELVYGSVSGKSFSWEDRGMALSMKDKDYNPLIGRTGVDVGRTFSGDDWKITAR
AGLGYQFDLLANGETVLRDASGEKRFEGEKDSRMLMNVGMNAEIKDNMRFGLELEKSAFGKYNVDNAINA
NFRYSF
SEQ ID NO: 15 - ESPC_ECO27 nucleic acid sequence encoding secretion unit
TATAAATCCT TCCTGACAGA GGTCAATAAT CTGAACAAGC GTATGGGTGA CCTGCGGGAT 60
ACTCAGGGGG ATGCCGGCGT CTGGGCGCGC ATCATGAACG GTACCGGTTC GGCAGATGGT 120
GGTTACAGCG ATAACTACAC TCACGTTCAG ATTGGTGCCG ACAGAAAGCA TGAGCTGGAC 180
GGTGTGGATT TGTTCACGGG TGCATTACTG ACCTATACAG ACAGCAATGC AAGCAGCCAC 240
GCCTTCAGTG GTAAAACCAA ATCCGTGGGG GGAGGGTTGT ACGCTTCAGC ACTCTTTGAT 300
TCCGGGGCTT ATTTTGACCT GATTGGTAAA TATCTCCATC ACGACAATCA GTACACGGCG 360
AGTTTTGCGT CTCTTGGTAC AAAAGACTAC AGCTCTCATT CCTGGTATGC CGGTGCAGAG 420
GTCGGGTATC GTTACCACCT GTCGGAAGAG TCCTGGGTGG AGCCACAGAT GGAGCTGGTT 480
TACGGTTCTG TGTCAGGAAA ATCTTTTAGC TGGGAAGACC GGGGAATGGC CCTGAGCATG 540
AAAGACAAGG ATTATAACCC ACTGATTGGC CGTACCGGTG TTGACGTGGG AAGAACCTTC 600
TCCGGAGACG ACTGGAAAAT TACCGCGCGA GCCGGGCTGG GTTACCAGTT CGACCTGCTG 660
GCGAACGGAG AAACGGTTCT GCGGGATGCA TCCGGAGAGA AACGTTTTGA AGGTGAAAAG 720
GACAGCAGAA TGCTGATGAA TGTGGGGATG AATGCGGAAA TTAAGGATAA TATGCGTTTT 780
GGCTTGGAGC TGGAAAAATC GGCGTTCGGG AAATATAACG TGGACAATGC GATAAACGCT 840
AACTTCCGTT ATTCTTTCTG A
SEQ ID NO: 16 - TSH_E. coli amino acid sequence
MNRIYSLRYSAVARGFIAVSEFARKCVHKSVRRLCFPVLLLIPVLFSAGSLAGTVNNELGYQLFRDFAEN
KGMFRPGATNIAIYNKQGEFVGTLDKAAMPDFSAVDSEIGVATLINPQYIASVKHNGGYTNVSFGDGENR
YNIVDRNNAPSLDFHAPRLDKLVTEVAPTAVTAQGAVAGAYLDKERYPVFYRLGSGTQYIKDSNGQLTQM
GGAYSWLTGGTVGSLSSYQNGEMISTSSGLVFDYKLNGAMPIYGEAGDSGSPLFAFDTVQNKWVLVGVLT
AGNGAGGRGNNWAVIPLDFIGQKFNEDNDAPVTFRTSEGGALEWSFNSSTGAGALTQGTTTYAMHGQQGN
DLNAGKNLIFQGQNGQINLKDSVSQGAGSLTFRDNYTVTTSNGSTWTGAGIVVDNGVSVNWQVNGVKGDN
LHKIGEGTLTVQGTGINEGGLKVGDGKVVLNQQADNKGQVQAFSSVNIASGRPTVVLTDERQVNPDTVSW
GYRGGTLDVNGNSLTFHQLKAADYGAVLANNVDKRATITLDYALRADKVALNGWSESGKGTAGNLYKYNN
PYTNTTDYFILKQSTYGYFPTDQSSNATWEFVGHSQGDAQKLVADRFNTAGYLFHGQLKGNLNVDNRLPE
GVTGALVMDGAADISGTFTQENGRLTLQGHPVIHAYNTQSVADKLAASGDHSVLTQPTSFSQEDWENRSF
TFDRLSLKNTDFGLGRNATLNTTIQADNSSVTLGDSRVFIDKNDGQGTAFTLEEGTSVATKDADKSVFNG
TVNLDNQSVLNINDIFNGGIQANNSTVNISSDSAVLGNSTLTSTALNLNKGANALASQSFVSDGPVNISD
AALSLNSRPDEVSHTLLPVYDYAGSWNLKGDDARLNVGPYSMLSGNINVQDKGTVTLGGEGELSPDLTLQ
NQMLYSLFNGYRNIWSGSLNAPDATVSMTDTQWSMNGNSTAGNMKLNRTIVGFNGGTSPFTTLTTDNLDA
VQSAFVMRTDLNKADKLVINKSATGHDNSIWVNFLKKPSNKDTLDIPLVSAPEATADNLFRASTRVVGFS
DVTPILSVRKEDGKKEWVLDGYQVARNDGQGKAAATFMHISYNNFITEVNNLNKRMGDLRDINGEAGTWV
RLLNGSGSADGGFTDHYTLLQMGADRKHELGSMDLFTGVMATYTDTDASADLYSGKTKSWGGGFYASGLF
RSGAYFDVIAKYIHNENKYDLNFAGAGKQNFRSHSLYAGAEVGYRYHLTDTTFVEPQAELVWGRLQGQTF
NWNDSGMDVSMRRNSVNPLVGRTGVVSGKTFSGKDWSLTARAGLHYEFDLTDSADVHLKDAAGEHQINGR
KDSRMLYGVGLNARFGDNTRLGLEVERSAFGKYNTDDAINANIRYSF
SEQ ID NO: 17 - Secretion unit from TSH_E. coli
YNNFITEVNNLNKRMGDLRDINGEAGTWVRLLNGSGSADGGFTDHYTLLQMGADRKHELGSMDLFTGVMA
TYTDTDASADLYSGKTKSWGGGFYASGLFRSGAYFDVIAKYIHNENKYDLNFAGAGKQNFRSHSLYAGAE
VGYRYHLTDTTFVEPQAELVWGRLQGQTFNWNDSGMDVSMRRNSVNPLVGRTGVVSGKTFSGKDWSLTAR
AGLHYEFDLTDSADVHLKDAAGEHQINGRKDSRMLYGVGLNARFGDNTRLGLEVERSAFGKYNTDDAINA
NIRYSF
SEQ ID NO: 18 - TSH_E. coli nucleic acid sequence encoding secretion unit
TATAACAACT TCATCACTGA AGTTAACAAC CTGAACAAAC GCATGGGCGA TTTGAGGGAT 60
ATTAATGGCG AAGCCGGTAC GTGGGTGCGT CTGCTGAACG GTTCCGGCTC TGCTGATGGC 120
GGTTTCACTG ACCACTATAC CCTGCTGCAG ATGGGGGCTG ACCGTAAGCA CGAACTGGGA 180
AGTATGGACC TGTTTACCGG CGTGATGGCC ACCTACACTG ACACAGATGC GTCAGCAGAC 240
CTGTACAGCG GTAAAACAAA ATCATGGGGT GGTGGTTTCT ATGCCAGTGG TCTGTTCCGG 300
TCCGGCGCTT ACTTTGATGT GATTGCCAAA TATATTCACA ATGAAAACAA ATATGACCTG 360
AACTTTGCCG GAGCTGGTAA ACAGAACTTC CGCAGCCATT CACTGTATGC AGGTGCAGAA 420
GTCGGATACC GTTATCATCT GACAGATACG ACGTTTGTTG AACCTCAGGC GGAACTGGTC 480
TGGGGAAGAC TGCAGGGCCA AACATTTAAC TGGAACGACA GTGGAATGGA TGTCTCAATG 540
CGTCGTAACA GCGTTAATCC TCTGGTAGGC AGAACCGGCG TTGTTTCCGG TAAAACCTTC 600
AGTGGTAAGG ACTGGAGTCT GACAGCCCGT GCCGGCCTGC ATTATGAGTT CGATCTGACG 660
GACAGTGCTG ACGTTCATCT GAAGGATGCA GCGGGAGAAC ATCAGATTAA TGGCAGAAAA 720
GACAGTCGTA TGCTTTACGG TGTGGGGTTA AATGCCCGGT TTGGCGACAA TACGCGTTTG 780
GGGCTGGAAG TTGAACGCTC TGCATTTGGT AAATACAACA CAGATGATGC GATAAACGCT 840
AATATTCGTT ATTCATTCTG A
SEQ ID NO: 19 - SEPA_EC536 amino acid sequence
MNKIYALKYCYITNTVKVVSELARRVCKGSTRRGKRLSVLTSLALSALLPTVAGASTVGGNNPYQTYRDF
AENKGQFQAGATNIPIFNNKGELVGHLDKAPMVDFSSVNVSSNPGVATLINPQYIASVKHNKGYQSVSFG
DGQNSYHIVDRNEHSSSDLHTPRLDKLVTEVAPATVTSSSTADILTPSKYSAFYRAGSGSQYIQDSQGKR
HWVTGGYGYLTGGILPTSFFYHGSDGIQLYMGGNIHDHSILPSFGEAGDSGSPLFGWNTAKGQWELVGVY
SGVGGGTNLIYSLIPQSFLSQIYSEDNDAPVFFNASSGAPLQWKFDSSTGTGSLKQGSDEYAMHGQKGSD
LNAGKNLTFLGHNGQIDLENSVTQGAGSLTFTDDYTVTTSNGSTWTGAGIIVDKDASVNWQVNGVKGDNL
HKIGEGTLVVQGTGVNEGGLKVGDGTVVLNQQADSSGHVQAFSSVNIASGRPTVVLADNQQVNPDNISWG
YRGGVLDVNGNDLTFHKLNAADYGATLGNSSDKTANITLDYQTHPADVKVNEWSSSNRGTVGSLYIYNNP
YTHTVDYFILKTSSYGWFPTGQVSNEHWEYVGHDQNSAQALLANRINNKGYLYHGKLLGNINFSNKATPG
TTGALVMDGSANMSGTFTQENGRLTIQGHPVIHASTSQSIANTVSSLGDNSVLTQPTSFTQDDWENRTFS
FGSLVLKDTDFGLGRNATLNTTIQADNSSVTLGDSRVFIDKKDGQGTAFTLEEGTSVATKDADKSVFNGT
VNLDNQSVLNINDIFNGGIQANNSTVNISSDSAILGNSTLTSTALNLNKGANALASQSFVSDGPVNISDA
TLSLNSRPDEVSHTLLPVYDYAGSWNLKGDDARLNVGPYSMLSGNINVQDKGTVTLGGEGELSPDLTLQN
QMLYSLFNGYRNTWSGSLNAPDATVSMTDTQWSMNGNSTAGNMKLNRTIVGFNGGTSSFTTLTTDNLDAV
QSAFVMRTDLNKADKLVINKSATGHDNSIWVNFLKKPSDKDTLDIPLVSAPEATADNLFRASTRVVGFSD
VTPTLSVRKEDGKKEWVLDGYQVARNDGQGKAAATFMHISYNNFITEVNNLNKRMGDLRDINGEAGTWVR
LLNGSGSADGGFTDHYTLLQMGADRKHELGSMDLFTGVMATYTDTDASAGLYSGKTKSWGGGFYASGLFR
SGAYFDLIAKYIHNENKYDLNFAGAGKQNFRSHSLYAGAEVGYRYHLTDTTFVEPQAELVWGRLQGQTFN
WNDSGMDVSMRRNSVNPLVGRTGVVSGKTFSGKDWSLTARAGLHYEFDLTDSADVHLKDAAGEHQINGRK
DGRMLYGVGLNARFGDNTRLGLEVERSAFGKYNTDDAINANIRYSF
SEQ ID NO: 20 - Secretion unit from SEPA_EC536
YNNFITEVNNLNKRMGDLRDINGEAGTWVRLLNGSGSADGGFTDHYTLLQMGADRKHELGSMDLFTGVMA
TYTDTDASAGLYSGKTKSWGGGFYASGLFRSGAYFDLIAKYIHNENKYDLNFAGAGKQNFRSHSLYAGAE
VGYRYHLTDTTFVEPQAELVWGRLQGQTFNWNDSGMDVSMRRNSVNPLVGRTGVVSGKTFSGKDWSLTAR
AGLHYEFDLTDSADVHLKDAAGEHQINGRKDGRMLYGVGLNARFGDNTRLGLEVERSAFGKYNTDDAINA
NIRYSF
SEQ ID NO: 21 - SEPA_EC536 nucleic acid sequence encoding secretion unit
TATAACAACT TCATCACTGA AGTTAACAAC CTGAACAAAC GCATGGGCGA TTTGAGGGAT 60
ATTAACGGCG AAGCCGGTAC GTGGGTGCGT CTGCTGAACG GTTCCGGCTC TGCTGATGGC 120
GGTTTCACTG ACCACTATAC CCTGCTGCAG ATGGGGGCTG ACCGTAAGCA CGAACTGGGA 180
AGTATGGACC TGTTTACCGG CGTGATGGCC ACCTACACTG ACACAGATGC GTCAGCAGGC 240
CTGTACAGCG GTAAAACAAA ATCATGGGGT GGTGGTTTCT ATGCCAGTGG TCTGTTCCGG 300
TCCGGCGCTT ACTTTGATTT GATTGCCAAA TATATTCACA ATGAAAACAA ATATGACCTG 360
AACTTTGCCG GAGCTGGTAA ACAGAACTTC CGCAGCCATT CACTGTATGC AGGTGCAGAA 420
GTCGGATACC GTTATCATCT GACAGATACG ACGTTTGTTG AACCTCAGGC GGAACTGGTC 480
TGGGGAAGAC TGCAGGGCCA AACATTTAAC TGGAACGACA GTGGAATGGA TGTCTCAATG 540
CGTCGTAACA GCGTTAATCC TCTGGTAGGC AGAACCGGCG TTGTTTCCGG TAAAACCTTC 600
AGTGGTAAGG ACTGGAGTCT GACAGCCCGT GCCGGCCTAC ATTATGAGTT CGATCTGACG 660
GACAGTGCTG ACGTTCACCT GAAGGATGCA GCGGGAGAAC ATCAGATTAA TGGGAGAAAA 720
GACGGTCGTA TGCTTTACGG TGTGGGGTTA AATGCCCGGT TTGGCGACAA TACGCGTCTG 780
GGGCTGGAAG TTGAACGCTC TGCATTCGGT AAATACAACA CAGATGATGC GATAAACGCT 840
AACATTCGTT ATTCATTCTG A
SEQ ID NO: 22 - PIC_ECO44 amino acid sequence
MNKVYSLKYCPVTGGLIAVSELARRVIKKTCRRLTHILLAGIPAICLCYSQISQAGIVRSDIAYQIYRDF
AENKGLFVPGANDIPVYDKDGKLVGRLGKAPMADFSSVSSNGVATLVSPQYIVSVKHNGGYRSVSFGNGK
NTYSLVDRNNHPSIDFHAPRLNKLVTEVIPSAVTSEGTKANAYKYTERYTAFYRVGSGTQYTKDKDGNLV
KVAGGYAFKTGGTTGVPLISDATIVSNPGQTYNPVNGPLPDYGAPGDSGSPLFAYDKQQKKWVIVAVLRA
YAGINGATNWWNVIPTDYLNQVMQDDFDAPVDFVSGLGPLNWTYDKTSGTGTLSQGSKNWTMHGQKDNDL
NAGKNLVFSGQNGAIILKDSVTQGAGYLEFKDSYTVSAESGKTWTGAGIITDKGTNVTWKVNGVAGDNLH
KLGEGTLTINGTGVNPGGLKTGDGIVVLNQQADTAGNIQAFSSVNLASGRPTVVLGDARQVNPDNISWGY
RGGKLDLNGNAVTFTRLQAADYGAVITNNAQQKSQLLLDLKAQDTNVSEPTIGNISPFGGTGTPGNLYSM
ILNSQTRFYILKSASYGNTLWGNSLNDPAQWEFVGMDKNKAVQTVKDRILAGRAKQPVIFHGQLTGNMDV
AIPQVPGGRKVIFDGSVNLPEGTLSQDSGTLIFQGHPVIHASISGSAPVSLNQKDWENRQFTMKTLSLKD
ADFHLSRNASLNSDIKSDNSHITLGSDRAFVDKNDGTGNYVIPEEGTSVPDTVNDRSQYEGNITLNHNSA
LDIGSRFTGGIDAYDSAVSITSPDVLLTAPGAFAGSSLTVHDGGHLTALNGLFSDGHIQAGKNGKITLSG
TPVKDTANQYAPAVYLTDGYDLTGDNAALEITRGAHASGDIHASAASTVTIGSDTPAELASAETAASAFA
GSLLEGYNAAFNGAITGGRADVSMHNALWTLGGDSAIHSLTVRNSRISSEGDRTFRTLTVNKLDATGSDF
VLRTDLKNADKINVTEKATGSDNSLNVSFMNNPAQGQALNIPLVTAPAGTSAEMFKAGTRVTGFSRVTPT
LHVDTSGGNTKWILDGFKAEADKAAAAKADSFMNAGYKNFMTEVNNLNKRMGDLRDTNGDAGAWARIMSG
AGSADGGYSDNYTHVQVGFDKKHELDGVDLFTGVTMTYTDSSADSHAFSGKTKSVGGGLYASALFESGAY
IDLIGKYIHHDNDYTGNFASLGTKHYNTHSWYAGAETGYRYHLTEDTFIEPQAELVYGAVSGKTFRWKDG
DMDLSMKNRDFSPLVGRTGVELGKTFSGKDWSVTARAGTSWQFDLLNNGETVLRDASGEKRIKGEKDSRM
LFNVGMNAQIKDNMRFGLEFEKSAFGKYNVDNAVNANFRYMF
SEQ ID NO: 23 - Secretion unit from PIC_ECO44
YKNFMTEVNNLNKRMGDLRDTNGDAGAWARIMSGAGSADGGYSDNYTHVQVGFDKKHELDGVDLFTGVTM
TYTDSSADSHAFSGKTKSVGGGLYASALFESGAYIDLIGKYIHHDNDYTGNFASLGTKHYNTHSWYAGAE
TGYRYHLTEDTFIEPQAELVYGAVSGKTFRWKDGDMDLSMKNRDFSPLVGRTGVELGKTFSGKDWSVTAR
AGTSWQFDLLNNGETVLRDASGEKRIKGEKDSRMLFNVGMNAQIKDNMRFGLEFEKSAFGKYNVDNAVNA
NFRYMF
SEQ ID NO: 24 - PIC_ECO44 nucleic acid sequence encoding secretion unit
TATAAAAACT TCATGACGGA AGTTAACAAT CTGAACAAAC GTATGGGTGA CCTGCGTGAC 60
ACAAACGGTG ATGCCGGTGC CTGGGCGCGC ATCATGAGTG GTGCCGGTTC TGCAGACGGT 120
GGTTACAGTG ATAATTACAC CCATGTTCAG GTCGGCTTTG ACAAAAAACA TGAACTGGAC 180
GGTGTGGACC TGTTTACCGG TGTCACGATG ACCTATACCG ACAGCAGTGC AGACAGCCAT 240
GCATTCAGCG GAAAGACGAA ATCGGTGGGG GGCGGTCTGT ATGCTTCAGC ATTGTTTGAG 300
TCCGGTGCCT ATATCGATTT GATTGGTAAA TATATTCACC ATGACAATGA TTACACAGGT 360
AACTTTGCTA GCCTGGGAAC GAAACACTAC AACACCCATT CCTGGTATGC CGGTGCTGAA 420
ACGGGTTACC GCTATCACCT GACAGAGGAC ACGTTCATTG AGCCGCAGGC TGAACTGGTT 480
TACGGCGCCG TGTCCGGGAA AACATTCCGC TGGAAAGACG GTGATATGGA CCTGAGCATG 540
AAGAACAGGG ACTTCAGTCC GCTGGTTGGA AGAACAGGGG TTGAACTGGG CAAGACCTTC 600
AGTGGTAAGG ACTGGAGTGT GACGGCCCGT GCCGGAACCA GCTGGCAGTT TGACCTGCTG 660
AATAATGGAG AGACCGTACT GCGTGATGCG TCCGGGGAGA AACGGATAAA AGGAGAGAAG 720
GACAGCCGGA TGCTGTTTAA TGTTGGTATG AATGCGCAGA TAAAGGACAA TATGCGCTTT 780
GGTCTGGAGT TTGAGAAGTC AGCCTTTGGT AAATATAACG TGGATAATGC GGTAAACGCG 840
AATTTCCGGT ATATGTTCTG A
SEQ ID NO: 25 - SEPA_SHIFL amino acid sequence
MNKIYYLKYCHITKSLIAVSELARRVTCKSHRRLSRRVILTSVAALSLSSAWPALSATVSAEIPYQIFRD
FAENKGQFTPGTTNISIYDKQGNLVGKLDKAPMADFSSATITTGSLPPGDHTLYSPQYVVTAKHVSGSDT
MSFGYAKNTYTAVGTNNNSGLDIKTRRLSKLVTEVAPAEVSDIGAVSGAYQAGGRFTEFYRLGGGMQYVK
DKNGNRTQVYTNGGFLVGGTVSALNSYNNGQMITAQTGDIFNPANGPLANYLNMGDSGSPLFAYDSLQKK
WVLIGVLSSGTNYGNNWVVTTQDFLGQQPQNDFDKTIAYTSGEGVLQWKYDAANGTGTLTQGNTTWDMHG
KKGNDLNAGKNLLFTGNNGEVVLQNSVNQGAGYLQFAGDYRVSALNGQTWMGGGIITDKGTHVLWQVNGV
AGDNLHKTGEGTLTVNGTGVNAGGLKVGDGTVILNQQADADGKVQAFSSVGIASGRPTVVLSDSQQVNPD
NISWGYRGGRLELNGNNLTFTRLQAADYGAIITNNSEKKSTVTLDLQTLKASDINVPVNTVSIFGGRGAP
GDLYYDSSTKQYFILKASSYSPFFSDLNNSSVWQNVGKDRNKAIDTVKQQKIEASSQPYMYHGQLNGNMD
VNIPQLSGKDVLALDGSVNLPEGSITKKSGTLIFQGHPVIHAGTTTSSSQSDWETRQFTLEKLKLDAATF
HLSRNGKMQGDINATNGSTVILGSSRVFTDRSDGTGNAVFSVEGSATATTVGDQSDYSGNVTLENKSSLQ
IMERFTGGIEAYDSTVSVTSQNAVFDRVGSFVNSSLTLGKGAKLTAQSGIFSTGAVDVKENASLTLTGMP
SAQKQGYYSPVISTTEGINLEDNASFSVKNMGYLSSDIHAGTTAATINLGDSDADAGKTDSPLFSSLMKG
YNAVLRGSITGAQSTVNMINALWYSDGKSEAGALKAKGSRIELGDGKHFATLQVKELSADNTTFLMHTNN
SRADQLNVTDKLSGSNNSVLVDFLNKPASEMSVTLITAPKGSDEKTFTAGTQQIGFSNVTPVISTEKTDD
ATKWVLTGYQTTADAGASKAAKDFMASGYKSFLTEVNNLNKRMGDLRDTQGDAGVWARIMNGTGSADGDY
SDNYTHVQIGVDRKHELDGVDLFTGALLTYTDSNASSHAFSGKNKSVGGGLYASALFNSGAYFDLIGKYL
HHDNQHTANFASLGTKDYSSHSWYAGAEVGYRYHLTKESWVEPQIELVYGSVSGKAFSWEDRGMALSMKD
KDYNPLIGRTGVDVGRAFSGDDWKITARAGLGYQFDLLANGETVLQDASGEKRFEGEKDSRMLMTVGMNA
EIKDNMRLGLELEKSAFGKYNVDNAINANFRYVF
SEQ ID NO: 26 - Secretion unit from SEPA_SHIFL
YKSFLTEVNNLNKRMGDLRDTQGDAGVWARIMNGTGSADGDYSDNYTHVQIGVDRKHELDGVDLFTGALL
TYTDSNASSHAFSGKNKSVGGGLYASALFNSGAYFDLIGKYLHHDNQHTANFASLGTKDYSSHSWYAGAE
VGYRYHLTKESWVEPQIELVYGSVSGKAFSWEDRGMALSMKDKDYNPLIGRTGVDVGRAFSGDDWKITAR
AGLGYQFDLLANGETVLQDASGEKRFEGEKDSRMLMTVGMNAEIKDNMRLGLELEKSAFGKYNVDNAINA
NFRYVF
SEQ ID NO: 27 - SEPA_SHIFL nucleic acid sequence encoding secretion unit
TATAAGTCCT TCCTTACAGA GGTCAATAAC CTGAACAAAC GTATGGGTGA CCTGCGGGAT 60
ACTCAGGGGG ATGCCGGTGT CTGGGCACGC ATAATGAATG GTACCGGTTC GGCAGATGGT 120
GACTACAGCG ATAACTACAC TCACGTTCAG ATTGGTGTCG ACAGAAAGCA TGAGCTGGAC 180
GGTGTGGATT TATTTACGGG GGCATTGCTG ACCTATACGG ACAGCAATGC AAGCAGCCAC 240
GCATTCAGTG GAAAAAACAA ATCCGTGGGT GGCGGTCTGT ATGCCTCTGC ACTCTTTAAT 300
TCCGGAGCTT ATTTTGACCT GATTGGTAAA TATCTCCATC ATGATAATCA GCACACGGCG 360
AATTTTGCCT CACTGGGAAC AAAAGACTAC AGCTCTCATT CCTGGTATGC CGGTGCTGAA 420
GTTGGTTATC GTTACCACCT GACGAAAGAG TCCTGGGTGG AGCCACAGAT AGAGCTGGTT 480
TACGGTTCTG TATCAGGAAA AGCTTTTAGC TGGGAAGACC GGGGAATGGC TCTGAGCATG 540
AAAGACAAGG ATTATAACCC ACTGATTGGC CGTACTGGTG TTGACGTGGG AAGAGCCTTC 600
TCCGGAGACG ACTGGAAAAT CACAGCTCGA GCCGGGCTGG GTTATCAGTT CGACCTGCTG 660
GCGAACGGAG AAACGGTTCT GCAGGATGCT TCCGGAGAGA AACGTTTCGA AGGTGAAAAA 720
GATAGCAGGA TGCTGATGAC GGTAGGGATG AATGCGGAAA TTAAGGATAA TATGCGTTTG 780
GGACTGGAGC TGGAGAAATC AGCGTTCGGG AAATATAATG TGGATAATGC GATAAACGCC 840
AACTTCCGTT ATGTTTTCTG A
SEQ ID NO: 28 - PET_ECO44 nucleic acid sequence encoding secretion unit,
optimised for expression in E. coli
TACAAAGCGT TCCTGGCGGA AGTTAACAAC CTGAACAAAC GTATGGGTGA CCTGCGTGAC 60
ATCAACGGTG AAGCGGGTGC GTGGGCGCGT ATCATGTCTG GCACCGGCTC GGCCGGTGGT 120
GGTTTCTCTG ACAACTACAC CCACGTTCAG GTTGGTGCGG ACAACAAACA CGAACTGGAC 180
GGTCTGGACC TGTTCACCGG CGTTACCATG ACCTACACCG ACTCTCACGC CGGCTCTGAC 240
GCTTTCTCTG GTGAAACCAA ATCTGTTGGT GCGGGTCTGT ACGCTTCTGC GATGTTTGAA 300
TCTGGTGCGT ACATCGACCT GATCGGTAAA TACGTTCACC ACGACAACGA ATACACCGCG 360
ACCTTCGCGG GTCTGGGTAC CCGTGACTAC TCTTCTCACT CTTGGTACGC GGGTGCGGAA 420
GTTGGTTACC GTTACCACGT TACCGACTCT GCGTGGATCG AACCGCAGGC GGAACTGGTT 480
TACGGTGCGG TTTCTGGTAA ACAGTTCTCT TGGAAAGACC AGGGTATGAA CCTGACCATG 540
AAAGACAAAG ACTTCAACCC GCTGATCGGT CGTACCGGCG TTGACGTCGG TAAATCTTTC 600
TCTGGTAAAG ACTGGAAAGT TACCGCGCGT GCGGGTCTGG GTTACCAGTT CGACCTGTTC 660
GCTAACGGTG AAACCGTTCT GCGTGACGCT TCTGGTGAAA AACGTATCAA AGGTGAAAAA 720
GACGGTCGTA TGCTGATGAA CGTGGGTCTG AACGCGGAAA TCCGTGACAA CGTGCGTTTC 780
GGTCTGGAAT TCGAGAAATC TGCGTTCGGT AAATACAACG TGGACAACGC GATCAACGCG 840
AACTTCCGTT ACTCTTTCTG ATAA
SEQ ID NO: 29: N-terminal signal sequence:
MNKIYSIKYSAATGGLIAVSELAKKVICKTNRKISAALLSLAVISYTNIIYA
SEQ ID NO: 30: Nucleic acid sequence encoding the N-terminal signal sequence:
ATGAATAAAA TATACTCCAT TAAATATAGT GCTGCCACTG GCGGACTCAT TGCTGTTTCT 60
GAATTAGCGA AAAAAGTCAT ATGTAAAACA AACCGAAAAA TTTCTGCTGC ATTATTATCT 120
CTGGCAGTTA TTAGTTATAC TAATATAATA TATGCC
SEQ ID NO: 31: Nucleic acid sequence encoding the N-terminal signal sequence
(codon optimised):
ATGAACAAAA TCTACTCTAT CAAATACTCT GCGGCGACCG GCGGTCTGAT CGCGGTTTCT 60
GAGCTCGCCA AAAAAGTTAT CTGCAAAACC AACCGTAAAA TCTCTGCGGC GCTGCTGTCT 120
CTGGCGGTTA TCTCTTACAC CAACATCATC TACGCG
SEQ ID NO: 32 - Secretion unit from PET_ECO44 without loop 3 amino acids
(1129-1136 according to the numbering used in SEQ ID NO: 1)
YKAFLAEVNNLNKRMGDLRDINGEAGAWARIMSGTGSAGGGFSDNYTHVQVGADNKHELDGLDLFTGVTM
TYTDSHAGSDAFSGETKSVGAGLYASAMFESGAYIDLIGKYVHHDNEYTRDYSSHSWYAGAEVGYRYHVT
DSAWIEPQAELVYGAVSGKQFSWKDQGMNLTMKDKDFNPLIGRTGVDVGKSFSGKDWKVTARAGLGYQFD
LFANGETVLRDASGEKRIKGEKDGRMLMNVGLNAEIRDNVRFGLEFEKSAFGKYNVDNAINANFRYSF

1. A bacterial expression construct comprising a nucleic acid sequence encoding a secretion unit peptide comprising less than 300 amino acids of the C-terminus of a SPATE-class bacterial autotransporter polypeptide, said secretion unit peptide comprising: (i) the α-helix; (ii) linker; and (iii) β-barrel region of the β-domain of the autotransporter polypeptide.

2. The expression construct of claim 1 wherein the α-helix, the linker and the β-barrel region are independently derivable from a SPATE-class bacterial autotransporter polypeptide selected front the following: Pet, Sat, EspP, SigA, EspC, Tsh, SepA, Pic, Hbp, SsaA, EatA, EpeA, EspI, PicU, Vat, Boa, IgA1, Hap, App, MspA, EaaA and EaaC.

3. The expression construct of claim 1 wherein the secretion unit peptide is derivable from a single SPATE-class bacterial autotransporter polypeptide.

4. The expression construct of claim 1 wherein the secretion unit peptide is derivable from a SPATE-class bacterial autotransporter polypeptide selected from the following: Pet, Sat, EspP, SigA, EspC, Tsh, SepA and Pic.

5. The expression construct of claim 1 wherein the secretion unit peptide is derivable form one or more SPATE-class bacterial autotransporter polypeptides selected from the following: PET_ECO44, SAT_CFT073, ESPP_ECO57, SIGA_SHIFL, ESPC_ECO27, TSH_{—}E. coli, SEPA_EC536, PIC_ECO44, SEPA_SHIFL.

6. The expression construct of claim 1 wherein the secretion unit peptide comprises the amino acid sequence provided in any one of SEQ ID NOs 2, 5, 8, 11, 14, 17, 20, 23 or 25 or a variant thereof wherein the variant is capable of mediating the extracellular secretion of a peptide tram the periplasm.

7. The expression construct of claim 1 wherein the nucleic acid sequence encoding the secretion unit peptide comprises the nucleic acid sequence provided in any one of SEQ ID NOs 3, 6, 9, 12, 15, 18, 21, 24, 27 or 28 or a variant thereof, wherein the variant encodes a secretion unit peptide capable of mediating the extracellular secretion of a peptide from the periplasm.

8. The expression construct of claim 1 wherein the secretion unit peptide consists of less than 300 amino acids of the C-terminus of a SPATE-class bacterial autotransporter polypeptide, said secretion unit peptide comprising: (i) the α-helix; (ii) linker; and (iii) β-barrel region of the β-domain of the autotransporter polypeptide.

9. The expression construct of claim 1 wherein the secretion unit peptide consists of the amino, acid sequence provided in any one of any one of SEQ ID NOs 2, 5, 8, 11, 14, 17, 20, 23 or 26 or a variant thereof wherein the variant is capable of mediating the extracellular secretion of a peptide from the periplasm.

10. The expression construct of claim 9 wherein the secretion, unit peptide consists of the amino acid sequence provided in SEQ ID NO: 2.

11. The expression construct of claim 1 wherein the construct further comprises a multiple cloning site located 5′ to the nucleic acid sequence encoding the N-terminal amino acid of the secretion unit.

12. The expression construct of claim 1 wherein the construct further comprises a nucleic acid sequence encoding a bacterial inner membrane signal peptide.

13. The expression construct of claim 10 wherein the bacterial inner membrane signal peptide comprises the amino acid sequence provided in SEQ ID NO. 29.

14. The expression construct of claim 12 or 13 wherein the construct has the following structure: (i) nucleic acid encoding a bacterial inner membrane signal peptide, operatively linked at the 3′ with (ii) a multiple cloning site, operatively linked at the 3′ with (iii) nucleic acid encoding the secretion unit.

15. The expression construct of claim 1 wherein the construct further comprises a second nucleic acid sequence encoding a protein of interest located at the multiple cloning site, the second nucleic acid arranged such that the protein of interest is operatively linked with the secretion unit peptide.

16. The expression construct of claim 15 wherein the construct encodes a recombinant polypeptide having the following structure: (i) a bacterial inner membrane signal peptide, operatively linked at the C-terminus with (ii) a protein of interest, operatively linked at the C-terminus with (iii) the secretion unit peptide.

17. (canceled)

18. (canceled)

19. A recombinant peptide comprising a secretion unit peptide comprising less than 300 amino acids of the C-terminus of a SPATE-class bacterial autotransporter polypeptide, said secretion unit peptide comprising: (i) the α-helix; (ii) linker; and (iii) β-barrel region of the β-domain of the autotransporter polypeptide.

20. The recombinant peptide of claim 19 wherein the peptide comprises a secretion unit as defined in claim 1.

21. A nucleic acid molecule comprising a sequence encoding the recombinant peptide of claims 19.

22. (canceled)

23. (canceled)

24. Use of a secretion unit peptide comprising less than 300 amino acids of the C-terminus of a SPATE-class bacterial autotransporter polypeptide, said secretion unit peptide comprising: (i) the α-helix; (ii) linker; and (iii) β-barrel region of the β-domain of the autotransporter polypeptide for secretion of a polypeptide from a bacterial periplasm.

25. (canceled)

26. (canceled)

27. (canceled)

28. (canceled)

29. (canceled)
